SEQUENCE LISTING (40 sequences)

SEQ ID NO: 1 - DNA, 435 bp, Artificial Sequence
  Synthetic
aaatcccttaacgtgagttacgcgcgcgtcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 2 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatatgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 3 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaacaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 4 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgttaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 5 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctatcagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 6 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccggcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 7 - DNA, 434 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtgtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 8 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagttaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 9 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctatcaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 10 - DNA, 434 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgaaacaaaaaaaccaccgctaccagcggtggtttgtttgccggatcaagagctatcaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 11 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 12 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccagctctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 13 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggtttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 14 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagtagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 15 - DNA, 435 bp, Artificial Sequence
  Synthetic
aaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtaaccgtagtcgagccactacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgac

SEQ ID NO: 16 - DNA, 435 bp, Artificial Sequence; identical to SEQ ID NO: 9
  Synthetic

SEQ ID NO: 17 - DNA, 500 bp, Artificial Sequence
  Synthetic
tattgaagatccgcttcgatcaggggatgggctactggcgcatcaacttttcatcgcaatggcattactttgatttccgcgatgacgtttctttccagttagtcaaaatggctcaggcctgcaaggaagggaatgtcgccaacagcgaagagagttgggcaacggatgtgctggtggaggtgatcgcctcctgatgatgagccgctcccgatgtggtgtcgggagcggtattttctataaaacttaccgcaatatcaggccggatgcggctgcgccttatccggcccataaccccttacttcctcaaccccgcaaacgcagcccgaatctcttcctccggcagctggatcccgataaacaccatcgtgctatgcggtttttcatcgccccacggcctgtcccagtcggcgctgtagaggcgctggacgccctggaacagcaggcggttaggttcgccgtcaatccacagcatccctttgtaacgtagcagtttatccgcc

SEQ ID NO: 18 - DNA, 200 bp, Artificial Sequence
  Synthetic
cggcgtaccgcaacacttttgttgtgcgtaaggtgtgtaaaggcaaacgtttaccttgcgattttgcaggagctgaagttagggtctggagtgaaatggatcacccgttgcgggagtctcttccggctcccgcagccactccttattcagcgtctcactatcgccgagatactcaagcaaccaggttaacgcaggcgaca

SEQ ID NO: 19 - DNA, 4108 bp, Artificial Sequence
  Synthetic
gggaaataagagagaaaagaagagtaagaagaaatataagagccaccatggaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaattcctcgggtagcaccggtttgccaaagggggtggcgttgccccaccgcactgcttgtgtgcggttctcgcacgctagggatcctatctttggtaatcagatcattcccgacacagcaatcctgtccgtggtaccttttcatcacggttttggcatgttcacgactctcggctatttgatttgcggtttcagggtcgtacttatgtatcggttcgaggaagaactgtttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaattcggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataacccagaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctagacatcccttcagagtcccgggtagcataaccccttggggcctctaaacgggtcttgaggggttttttgcgagctcggtacccagccccgacgagcttcatgccgttagtcgcactgcaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtcttccgtcttgcacaagcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccattctatggaactgcctcggtgagttttctccttcattacagaaacggctttttcaaaaatatggtattgataatcctgatatgaataaattgcagtttcatttgatgctcgatgagtttttctaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctgctcgccgcagccgaacgaccgagcgcagcgagtcagtgagcgaggaagcggaaggcgagagtagggaactgccaggcatcaaactaagcagaaggcccctgacgcatggcctttttgcgtttctacaaactctttctgtgttgtaaaacgacggccagtcttaagctcgggccccttttccgccagggttttcccagtcacgacgaattcgatccggctcaagcttttggaccctcgtacagaagctaatacgactcactata

SEQ ID NO: 20 - DNA, 3047 bp, Artificial Sequence
  Synthetic
gggaaataagagagaaaagaagagtaagaagaaatataagagccaccatgggagtgcacgagtgtcccgcgtggttgtggttgctgctgtcgctcttgagcctcccactgggactgcctgtgctgggggcaccacccagattgatctgcgactcacgggtacttgagaggtaccttcttgaagccaaagaagccgaaaacatcacaaccggatgcgccgagcactgctccctcaatgagaacattactgtaccggatacaaaggtcaatttctatgcatggaagagaatggaagtaggacagcaggccgtcgaagtgtggcaggggctcgcgcttttgtcggaggcggtgttgcggggtcaggccctcctcgtcaactcatcacagccgtgggagcccctccaacttcatgtcgataaagcggtgtcggggctccgcagcttgacgacgttgcttcgggctctgggcgcacaaaaggaggctatttcgccgcctgacgcggcctccgcggcacccctccgaacgatcaccgcggacacgtttaggaagctttttagagtgtacagcaatttcctccgcggaaagctgaaattgtatactggtgaagcgtgtaggacaggggatcgctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctagacatcccttcagagtcccgggtagcataaccccttggggcctctaaacgggtcttgaggggttttttgcgagctcggtacccagccccgacgagcttcatgccgttagtcgcactgcaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtcttccgtcttgcacaagcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccattctatggaactgcctcggtgagttttctccttcattacagaaacggctttttcaaaaatatggtattgataatcctgatatgaataaattgcagtttcatttgatgctcgatgagtttttctaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctgctcgccgcagccgaacgaccgagcgcagcgagtcagtgagcgaggaagcggaaggcgagagtagggaactgccaggcatcaaactaagcagaaggcccctgacgcatggcctttttgcgtttctacaaactctttctgtgttgtaaaacgacggccagtcttaagctcgggccccttttccgccagggttttcccagtcacgacgaattcgatccggcaatctagaaatcaagcttttggaccctcgtacagaagctaatacgactcactata

SEQ ID NO: 21 - DNA, 4324 bp, Artificial Sequence
  Synthetic
gcagagcattacgctgacttgacgggacggcgcaagctcatgaccaaaatcccttaacgtgagttacgcgcgcgcttatgttttcgctgatatcccgagcggtttcaaaattgtgatctatatttaacaagcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgctgccaggcatcaaactaagcagaaggcccctgacgcatggcctttttgcgtttctacaaactctttctgtgttgtaaaacgacggccagtcttaagctcgggccccttttccgccagggttttcccagtcacgacgaattcgatccggctcaagcttttggaccctcgtacagaagctaatacgactcactatagggaaataagagagaaaagaagagtaagaagaaatataagagccaccatggaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaattcctcgggtagcaccggtttgccaaagggggtggcgttgccccaccgcactgcttgtgtgcggttctcgcacgctagggatcctatctttggtaatcagatcattcccgacacagcaatcctgtccgtggtaccttttcatcacggttttggcatgttcacgactctcggctatttgatttgcggtttcagggtcgtacttatgtatcggttcgaggaagaactgtttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaattcggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataaccctgaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctagacatcccttcagagtcccgggtagcataaccccttggggcctctaaacgggtcttgaggggttttttgcgagctcggtacccagccccgacgagcttcatgccgttagtcgcactgcaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtcttccgtcttgcacaagcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccattctatggaactgcctcggtgagttttctccttcattacagaaacggctttttcaaaaatatggtattgataatcctgatatgaataaattgcagtttcatttgatgctcgatgagtttttctaa

SEQ ID NO: 22 - DNA, 4324 bp, Artificial Sequence
  Synthetic
gcagagcattacgctgacttgacgggacggcgcaagctcatgaccaaaatcccttaacgtgagttacgcgcgcgcttatgttttcgctgatatcccgagcggtttcaaaattgtgatctatatttaacaagcaaacaaaaaaaccaccgctaccagcggtggtttgtttgccggatcaagagctaccaactctttttccgaaggtaactggcttcagcagagcgcagataccaaatactgttcttctagtgtaaccggagcaagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgctgccaggcatcaaactaagcagaaggcccctgacgcatggcctttttgcgtttctacaaactctttctgtgttgtaaaacgacggccagtcttaagctcgggccccttttccgccagggttttcccagtcacgacgaattcgatccggctcaagcttttggaccctcgtacagaagctaatacgactcactatagggaaataagagagaaaagaagagtaagaagaaatataagagccaccatggaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaattcctcgggtagcaccggtttgccaaagggggtggcgttgccccaccgcactgcttgtgtgcggttctcgcacgctagggatcctatctttggtaatcagatcattcccgacacagcaatcctgtccgtggtaccttttcatcacggttttggcatgttcacgactctcggctatttgatttgcggtttcagggtcgtacttatgtatcggttcgaggaagaactgtttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaattcggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataaccctgaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctagacatcccttcagagtcccgggtagcataaccccttggggcctctaaacgggtcttgaggggttttttgcgagctcggtacccagccccgacgagcttcatgccgttagtcgcactgcaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtcttccgtcttgcacaagcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccattctatggaactgcctcggtgagttttctccttcattacagaaacggctttttcaaaaatatggtattgataatcctgatatgaataaattgcagtttcatttgatgctcgatgagtttttctaa

SEQ ID NO: 23 - DNA, 948 bp, Artificial Sequence
  Synthetic
atgccagacatgaagctgtttgcgggtaacgcgacccctgagctggcccagcgtatcgcgaaccgcttgtacacgagcctgggtgacgcagcggttggccgtttcagcgatggtgaagtcagcgtgcagattaatgaaaatgtgcgtggtggcgacattttcatcattcagagcacctgtgcgccgacgaacgataacctgatggaattggttgtgatggtcgatgcactgcgtcgcgcctccgccggtcgcattaccgcggtgattccgtattttggctatgcacgccaggatcgtcgtgtccgctccgcgcgcgtcccgatcacggcgaaagtcgtcgcggattttctgagcagcgtgggtgttgaccgtgtgctgaccgtggcgttgcatgctgagcaaattcaaggtttcttcgacgtcccggtggataatgttttcggttctccgattcttctggaagatatgctgcaactgaatctggataatccgatcgtcgttagcccggatatcggtggcgtggtgcgtgcgcgtgcaattgcaaagctgctgaatgataccgacatggcaatcatcgacaagcgccgtccgcgtgcgaatgtcagccaagtcatgcacatcattggcgacgttgctggccgtgactgcgttttagtggacgacatgatcgatacgggtggcactctgtgtaaagccgctgaggccctgaaagagcgcggtgcgaaacgtgttttcgcatacgcgacgcacccgatctttagcggtaatgctgcgaacaacttgcgtaactctgttattgacgaagttgttgtttgcgacaccattccgctgagcgacgaaatcaagagcctgccgaacgtgcgtaccctgaccctgagcggcatgctcgcagaggccatcagacgtattagcaacgaagagtcgatcagcgcgatgtttgagcattga

SEQ ID NO: 24 - protein, 315 aa, Artificial Sequence
  Synthetic
MPDMKLFAGNATPELAQRIANRLYTSLGDAAVGRFSDGEVSVQINENVRGGDIFIIQSTCAPTNDNLMELVVMVDALRRASAGRITAVIPYFGYARQDRRVRSARVPITAKVVADFLSSVGVDRVLTVALHAEQIQGFFDVPVDNVFGSPILLEDMLQLNLDNPIVVSPDIGGVVRARAIAKLLNDTDMAIIDKRRPRANVSQVMHIIGDVAGRDCVLVDDMIDTGGTLCKAAEALKERGAKRVFAYATHPIFSGNAANNLRNSVIDEVVVCDTIPLSDEIKSLPNVRTLTLSGMLAEAIRRISNEESISAMFEH

SEQ ID NO: 25 - DNA, 1226 bp, Artificial Sequence
  Synthetic
cggcgtaccgcaacacttttgttgtgcgtaaggtgtgtaaaggcaaacgtttaccttgcgattttgcaggagctgaagttagggtctggagtgaaatggaatggcaacaataaaagatgtagcgaaacgagcaaacgtttccactacaactgtgtcacacgtgatcaacaaaacacgtttcgtcgctgaagaaacgcgcaacgccgtgtgggcagcgattaaagaattacactactcccctagcgcggtggcgcgtagcctgaaggttaaccacaccaagtctatcggtttgctggcgaccagcagcgaagcggcctattttgccgagatcattgaagcagttgaaaaaaattgcttccagaaaggttacaccctgattctgggcaatgcgtggaacaatcttgagaaacagcgggcttatctgtcgatgatggcgcaaaaacgcgtcgatggtctgctggtgatgtgttctgagtacccagagccgttgctggcgatgctggaagagtatcgccatatcccaatggtggtcatggactggggtgaagcaaaagctgacttcaccgatgcggtcattgataacgcgttcgaaggcggctacatggccgggcgttatctgattgaacgcggtcaccgcgaaatcggcgtcatccccggcccgctggaacgtaacaccggcgcaggccgccttgccggttttatgaaggcgatggaagaagcgatgatcaaggtgccggaaagctggattgtgcagggtgactttgaacctgaatccggttatcgcgccatgcagcaaatcctgtcgcagccgcatcgccctactgccgtcttctgtggtggcgatatcatggcaatgggcgcactttgtgctgctgatgaaatgggcctgcgcgtcccgcaggatgtttcgctgatcggttatgataacgtgcgcaacgcgcgctattttacgccggcgctgaccacgatccatcagccaaaagattcgctgggtgaaacagcgttcaacatgctgttggatcgtatcgtcaacaaacgtgaagaaccgcagtctattgaagtgcatccgcgcttgattgaacgccgctccgtggctgacggcccgttccgcgactatcgtcgttaatcacccgttgcgggagtctcttccggctcccgcagccactccttattcagcgtctcactatcgccgagatactcaagcaaccaggttaacgcaggcgaca

SEQ ID NO: 26 - DNA, 7146 bp, Artificial Sequence
  Synthetic
catcgatttattaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaattgctagcattgtacctaggactgagctagccataaagttgacactctatcgttgatagagttattttaccactccctatcagtgatagagaaagaattcaaaggatccaaacaggagacattaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaatcttcgttggtctcggctactccttcgctatcgtccacgctgaagtccggcgtactattagggttgctcagtaacagctcacgcactttcttctcaatctctttggctgtctcgggattgtccttcagccatgccgtggcattagctttgccttggccaatcttctctcctttgtaagagtaccaggcaccagccttttcaatcagcttttctttcacccccaaatctaccaattcgccgtagaaattaattccttctccatataaaatctggaactccgcctgcttaaacggggctgcgattttattctttactactttgacgcgggtctcactcccgacgacgttttcaccctctttgactgccccgatgcgacgaatatccaaacgaacggacgcatagaacttcagcgcattacctccggtagtggtttcaggattgccaaacataactccaatcttcatgcggatttgattgataaagataagaagcgtgttggattgcttcaggttccccgccaacttgcgcattgcctggctcatcatgcgggcagcaagccccatgtgactgtcaccaatctcgccctcgatctcagctttaggcgtcagagcggccactgaatctacgacaatgacatctactgcgcctgagcgagccagggcatcgcagatttcaagcgcctgctctcctgtatcaggctgggagcataaaagattgtcaatatcgacccccaacttgcgcgcatagatcggatcaagcgcgtgttctgcgtcgataaaagcacacgttttaccctcgcgctgcgcagccgcgattacttgtaaggtcaaagtagtcttgccggagctttccggaccgtagatctctacaatacgacccataggtaaaccgcctgcacctaaggcaatatccagcgaaagagacccggtgctgatggtctctacatccatagagcggtcctcccccaagcgcataatagatcccttgccaaattgtttctcgatttggcccagcgcggcggccaatgctttctgtttattttcatcgattgccatgattgatatcctttttactcctgtcatgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 27 - DNA, 57 bp, Artificial Sequence
  Synthetic
gcttatgttttcgctgatatcccgagcggtttcaaaattgtgatctatatttaacaa

SEQ ID NO: 28 - DNA, 363 bp, Artificial Sequence
  Synthetic
tgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctg

SEQ ID NO: 29 - DNA, 609 bp, Artificial Sequence
  Synthetic
gcaaacaaaaaaaccaccgctaccagcggtggtttgtttgccggatcaagagctaccaactctttttccgaaggtaactggcttcagcagagcgcagataccaaatactgttcttctagtgtaaccggagcaagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 30 - protein, 235 aa, Escherichia coli
MYRYLSIAAVVLSAAFSGPALAEGINSFSQAKAAAVKVHADAPGTFYCGCKINWQGKKGVVDLQSCGYQVRKNENRASRVEWEHVVPAWQFGHQRQCWQDGGRKNCAKDPVYRKMESDMHNLQPSVGEVNGDRGNFMYSQWNGGEGQYGQCAMKVDFKEKAAEPPARARGAIARTYFYMRDQYNLTLSRQQTQLFNAWNKMYPVTDWECERDERIAKVQGNHNPYVQRACQARKS

SEQ ID NO: 31 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 32 - protein, 304 aa, Artificial Sequence
  Synthetic
MTVPTYDKFIEPVLRYLATKPEGAAARDVHEAAADALGLDDSQRAKVITSGQLVYKNRAGWAHDRLKRAGLSQSLSRGKWCLTPAGFDWVASHPQPMTEQETNHLAFAFVNVKLKSRPDAVDLDPKADSPDHEELAKSSPDDRLDQALKELRDAVADEVLENLLQVSPSRFEVIVLDVLHRLGYGGHRDDLQRVGGTGDGGIDGVISLDKLGLEKVYVQAKRWQNTVGRPELQAFYGALAGQKAKRGVFITTSGFTSQARDFAQSVEGMVLVDGERLVHLMIENEVGVSSRLLKVPKLDMDYFE

SEQ ID NO: 33 - DNA, 1026 bp, Artificial Sequence
  Synthetic
atggcaacaataaaagatgtagcgaaacgagcaaacgtttccactacaactgtgtcacacgtgatcaacaaaacacgtttcgtcgctgaagaaacgcgcaacgccgtgtgggcagcgattaaagaattacactactcccctagcgcggtggcgcgtagcctgaaggttaaccacaccaagtctatcggtttgctggcgaccagcagcgaagcggcctattttgccgagatcattgaagcagttgaaaaaaattgcttccagaaaggttacaccctgattctgggcaatgcgtggaacaatcttgagaaacagcgggcttatctgtcgatgatggcgcaaaaacgcgtcgatggtctgctggtgatgtgttctgagtacccagagccgttgctggcgatgctggaagagtatcgccatatcccaatggtggtcatggactggggtgaagcaaaagctgacttcaccgatgcggtcattgataacgcgttcgaaggcggctacatggccgggcgttatctgattgaacgcggtcaccgcgaaatcggcgtcatccccggcccgctggaacgtaacaccggcgcaggccgccttgccggttttatgaaggcgatggaagaagcgatgatcaaggtgccggaaagctggattgtgcagggtgactttgaacctgaatccggttatcgcgccatgcagcaaatcctgtcgcagccgcatcgccctactgccgtcttctgtggtggcgatatcatggcaatgggcgcactttgtgctgctgatgaaatgggcctgcgcgtcccgcaggatgtttcgctgatcggttatgataacgtgcgcaacgcgcgctattttacgccggcgctgaccacgatccatcagccaaaagattcgctgggtgaaacagcgttcaacatgctgttggatcgtatcgtcaacaaacgtgaagaaccgcagtctattgaagtgcatccgcgcttgattgaacgccgctccgtggctgacggcccgttccgcgactatcgtcgttaa

SEQ ID NO: 34 - DNA, 1262 bp, Artificial Sequence
  Synthetic
caatttctacaaaacacttgatactgtatgagcatacagtataattgcttcaacagaacatattgactatccggtattacccggcatgacaggagtaaaaatggctatcgacgaaaacaaacagaaagcgttggcggcagcactgggccagattgagaaacaatttggtaaaggctccatcatgcgcctgggtgaagaccgttccatggatgtggaaaccatctctaccggttcgctttcactggatatcgcgcttggggcaggtggtctgccgatgggccgtatcgtcgaaatctacggaccggaatcttccggtaaaaccacgctgacgctgcaggtgatcgccgcagcgcagcgtgaaggtaaaacctgtgcgtttatcgatgctgaacacgcgctggacccaatctacgcacgtaaactgggcgtcgatatcgacaacctgctgtgctcccagccggacaccggcgagcaggcactggaaatctgtgacgccctggcgcgttctggcgcagtagacgttatcgtcgttgactccgtggcggcactgacgccgaaagcggaaatcgaaggcgaaatcggcgactctcacatgggccttgcggcacgtatgatgagccaggcgatgcgtaagctggcgggtaacctgaagcagtccaacacgctgctgatcttcatcaaccagatccgtatgaaaattggtgtgatgttcggtaacccggaaaccactaccggtggtaacgcgctgaaattctacgcctctgttcgtctcgacatccgtcgtatcggcgcggtgaaagagggcgaaaacgtggtgggtagcgaaacccgcgtgaaagtggtgaagaacaaaatcgctgcgccgtttaaacaggctgaattccagatcctctacggcgaaggtatcaacttctacggcgaactggttgacctgggcgtaaaagagaagctgatcgagaaagcaggcgcgtggtacagctacaaaggtgagaagatcggtcagggtaaagcgaatgcgactgcctggctgaaagataacccggaaaccgcgaaagagatcgagaagaaagtacgtgagttgctgctgagcaacccgaactcaacgccggatttctctgtagatgatagcgaaggcgtagcagaaactaacgaagatttttaatcgtcttgtttgatacacaagggtcgcatctgcggcccttttgcttttttaagttgtaaggatatgccatgacagaatcaacatcccgtcgcccggcata

SEQ ID NO: 35 - DNA, 200 bp, Artificial Sequence
  Synthetic
caatttctacaaaacacttgatactgtatgagcatacagtataattgcttcaacagaacatattgactatccggtattacccggcatgacaggagtaaaatcgtcttgtttgatacacaagggtcgcatctgcggcccttttgcttttttaagttgtaaggatatgccatgacagaatcaacatcccgtcgcccggcata

SEQ ID NO: 36 - DNA, 808 bp, Artificial Sequence
  Synthetic
aaataaccatctgaactatcaggaactttcctgatctggctgattgcataccaaaacagctttcgctacgttgctggctcgttttaacacggagtaagtgatgtaccgttatttgtctattgctgcggtggtactgagcgcagcattttccggcccggcgttggccgaaggtatcaatagtttttctcaggcgaaagccgcggcggtaaaagtccacgctgacgcgcccggtacgttttattgcggatgtaaaattaactggcagggcaaaaaaggcgttgttgatctgcaatcgtgcggctatcaggtgcgcaaaaatgaaaaccgcgccagccgcgtagagtgggaacatgtcgttcccgcctggcagttcggtcaccagcgccagtgctggcaggacggtggacgtaaaaactgcgctaaagatccggtctatcgcaagatggaaagcgatatgcataacctgcagccgtcagtcggtgaggtgaatggcgatcgcggcaactttatgtacagccagtggaatggcggtgaaggccagtacggtcaatgcgccatgaaggtcgatttcaaagaaaaagctgccgaaccaccagcgcgtgcacgcggtgccattgcgcgcacctacttctatatgcgcgaccaatacaacctgacactctctcgccagcaaacgcagctgttcaacgcatggaacaagatgtatccggttaccgactgggagtgcgagcgcgatgaacgcatcgcgaaggtgcagggcaatcataacccgtatgtgcaacgcgcttgccaggcgcgaaagagctaa

SEQ ID NO: 37 - DNA, 200 bp, Artificial Sequence
  Synthetic
aaataaccatctgaactatcaggaactttcctgatctggctgattgcataccaaaacagctttcgctacgttgctggctcgttttaacacggagtaagtgggttaccgactgggagtgcgagcgcgatgaacgcatcgcgaaggtgcagggcaatcataacccgtatgtgcaacgcgcttgccaggcgcgaaagagctaa

SEQ ID NO: 38 - DNA, 11152 bp, Artificial Sequence
  Synthetic
agagttgggcaacggatgtgctggtggaggtgatcgcctcctgatgatgagccgctcccgatgtggtgtcgggagcggtattttctataaaacttaccgctcactcaaaatagtccatatccagtttcggcaccttcaacaaacgtgaagaaacccctacttcgttttcgatcattaagtgcaccaggcgttccccatcaaccaacaccataccctcgacggattgggcaaagtcacgcgcctgagaagtaaatccagaagtggtaataaacaccccacgtttcgctttttgcccagccagtgcgccgtaaaatgcctgtaattctggcctgcctacagtattctgccaacgttttgcctgaacataaactttctccaggccaagtttatcaagcgatatcacaccatcgatgccaccatctccagtaccgccaacacgctgcaaatcatcacggtggccgccataccccaggcgatgcaaaacatccagaacaatgacttcaaagcgcgaaggagaaacctgcaataagttttccagaacctcatcagccaccgcatcacgaagctcttttagcgcctgatctaaccgatcgtccgggctgctctttgcaagttcttcatgatcgggagagtcggctttcggatctaaatcgacggcatccggccgtgacttaagtttgacattcacaaaagcgaaggccagatggttcgtctcctgctccgtcattggctggggatgagacgcaacccagtcaaaacccgcaggagtcaggcaccatttgccacgcgacaaactttgcgacaacccggcacgttttaaacggtcatgcgcccagcctgcacgatttttataaacaagttgtccgctggtaatgactttcgctcgctggctgtcatccagtcctaatgcatccgcggcagcctcatgaacatcacgcgcggctgcaccttccggttttgttgccagataacgcagaacaggttcaataaatttgtcataggtaggaaccgtcatagtacatccttgcagaatcaggtagatgtttttcggctactatagcactacaaaaatagacgaacacgttagaaatgagtcagttgctgtgaccgtggtcattgcccggaaaggtacagaaagctaagatgagatgttatgggccttaaatatttggacaggcccgcacagcaatggattaataacaatgatgaataaatccaattttgaattcctgaagggcgtcaacgacttcacttatgccatcgcctgtgcggcggaaaataactacccggatgatcccaacacgacgctgattaaaatgcgtatgtttggcgaagccacagcgaaacatcttggtctgttactcaacatccccccttgtgagaatcaacacgatctcctgcgtgaactcggcaaaatcgcctttgttgatgacaacatcctctctgtatttcacaaattacgccgcattggtaaccaggcggtgcacgaatatcataacgatctcaacgatgcccagatgtgcctgcgactcgggttccgcctggctgtctggtactaccgtctggtcactaaagattatgacttcccggtgccggtgtttgtgttgccggaacgtggtgaaaacctctatcaccaggaagtgctgacgctaaaacaacagcttgaacagcaggtgcgagaaaaagcgcagactcaggcagaagtcgaagcgcaacagcagaagctggttgccctgaacggctatatcgccattctggaaggcaaacagcaggaaaccgaagcgcaaacccaggctcgccttgcggcactggaagcacagctcgccgagaagaacgcggaactggcaaaacagaccgaacaggaacgtaaggcttaccacaaagaaattaccgatcaggccatcaagcgcacactcaaccttagcgaagaagagagtcgcttcctgattgatgcgcaactgcgtaaagcaggctggcaggccgacagcaaaaccctgcgcttctccaaaggcgcacgtccggaacccggcgtcaataaagccattgccgaatggccgaccggaaaagatgaaacgggtaatcagggctttgcggattatgtgctgtttgtcggcctcaaacccatcgcggtggtagaggcgaaacgtaacaatatcgacgttcccgccaggctcaatgagtcgtatcgctacagtaaatgtttcgataatggcttcctgcgggaaaccttgcttgagcactactcaccggatgaagtgcatgaagcagtgccagagtatgaaaccagctggcaggacaccagcggcaaacaacggtttaaaatccccttctgctactcgaccaacgggcgcgaataccgcgcaacaatgaagaccaaaagcggcatctggtatcgcgacgtgcgtgatacccgcaatatgtcgaaagccttacccgagtggcaccgcccggaagagctgctggaaatgctcggcagcgaaccgcaaaaacagaatcagtggtttgccgataaccctggcatgagcgagctgggcctgcgttattatcaggaagatgccgtccgcgcggttgaaaaggcaatcgtcaaggggcaacaagagatcctgctggcgatggcgaccggtaccggtaaaacccgtacggcaatcgccatgatgttccgcctgatccagtcccagcgttttaaacgcattctcttccttgtcgaccgccgttctcttggcgaacaggcgctgggcgcgtttgaagatacgcgtattaacggcgacaccttcaacagcattttcgacattaaagggctgacggataaattcccggaagacagcaccaaaattcacgttgccaccgtacagtcgctggtgaaacgcaccctgcaatcagatgaaccgatgccggtggcccgttacgactgtatcgtcgttgacgaagcgcatcgcggctatattctcgataaagagcagaccgaaggcgaactgcagttccgcagccagctggattacgtctctgcctaccgtcgcattctcgatcacttcgatgcggtaaaaatcgctctcaccgccaccccggcgctacatactgtgcagattttcggcgagccggtttaccgttatacctaccgtaccgcggttatcgacggttttctgatcgaccaggatccgcctattcagatcatcacccgcaacgcgcaggagggggtttatctctccaaaggcgagcaggtagagcgcatcagcccgcagggagaagtgatcaatgacaccctggaagacgatcaggattttgaagtcgccgactttaaccgtggcctggtgatcccggcgtttaaccgcgccgtctgtaacgaactcaccaattatcttgacccgaccggatcgcaaaaaacgctggtcttctgcgtcaccaatgcccatgccgatatggtggtggaagagctgcgtgccgcgttcaagaaaaagtatccgcaactggagcacgacgcgatcatcaagatcaccggtgatgccgataaagacgcgcgcaaagtgcagaccatgatcacccgcttcaataaagagcggctgcccaatatcgtggtaaccgtcgacctgctgacgaccggcgtcgatattccgtcgatctgtaatatcgtgttcctgcgtaaagtacgcagccgcattctgtacgaacagatgaaaggccgcgccacgcgcttatgcccggaggtgaataaaaccagctttaagatttttgactgtgtcgatatctacagcacgctggagagcgtcgacaccatgcgtccggtggtggtgcgcccgaaggtggaactgcaaacgctggtcaatgaaattaccgattcagaaacctataaaatcaccgaagcggatggccgcagttttgccgagcacagccatgaacaactggtggcgaagctccagcgtatcatcggtctggccacgtttaaccgtgaccgcagcgaaacgatagataaacaggtgcgtcgtctggatgagctatgccaggacgcggcgggcgtgaactttaacggcttcgcctcgcgcctgcgggaaaaagggccgcactggagcgccgaagtctttaacaaactgcctggctttatcgcccgtctggaaaagctgaaaacggacatcaacaacctgaatgatgcgccgatcttcctcgatatcgacgatgaagtggtgagtgtaaaatcgctgtacggtgattacgacacgccgcaggatttcctcgaagcctttgactcgctggtgcaacgttccccgaacgcgcaaccggcattgcaggcagttattaatcgcccgcgcgatctcacccgtaaagggctggtcgagctacaggagtggtttgaccgccagcactttgaggaatcttccctgcgcaaagcatggaaagagacgcgcaatgaagatatcgccgcccggctgattggtcatattcgccgcgctgcggtgggcgatgcgctgaaaccgtttgaggaacgtgtcgatcacgcgctgacgcgcattaagggcgaaaacgactggagcagcgagcaattaagctggctcgatcgtttagcgcaggcgctgaaagagaaagtggtgctcgacgacgatgtcttcaaaaccggcaacttccaccgtcgcggcgggaaggcgatgctgcaaagaacctttgacgataatctcgataccctgctgggcaaattcagcgattatatctgggacgagctggcctgacacgtatacacttcatccttcaggctgcctctgcgttggctgcgctcgttcaccccggtcacgtacttctgtacgctcccggggattcactcacttgccgccttgatgcaacctgaatgattttgtgtatattaccctcggcaatttcttcttctgcggctcgatgaatttgggccgctgcttaatttacggaactcacaatgaacaataacgatctggtcgcgaagctgtggaagctgtgcgacaacctgcgcgatggcggcgtttcctatcaaaactacgtcaatgaactcgcctcgctgctgtttttgaaaatgtgtaaagagaccggtcaggaagcggaatacctgccggaaggttaccgctgggatgacctgaaatcccgcatcggccaggagcagttgcagttctaccgaaaaatgctcgtgcatttaggcgaagatgacaaaaagctggtacaggcagtttttcataatgttagtaccaccatcaccgagccgaaacaaataaccgcactggtcagcaatatggattcgctggactggtacaacggcgcgcacggtaagtcgcgcgatgacttcggcgatatgtacgaagggctgttgcagaagaacgcgaatgaaaccaagtctggtgcaggccagtacttcaccccgcgtccgctgattaaaaccattattcatctgctgaaaccgcagccgcgtgaagtggtgcaggacccggcggcaggtacggcgggctttttgattgaagccgaccgctatgttaagtcgcaaaccaatgatctggacgaccttgatggcgacacgcaggatttccagatccaccgcgcgtttatcggcctcgaactggtgcccggcacccgtcgtctggcactgatgaactgcctgctgcacgatattgaaggcaacctcgaccacggcggcgcaatccgtctgggcaacactctgggtagcgacggtgaaaacctgccgaaggcgcatattgtcgccactaacccgccgtttggcagcgccgcaggcaccaacattacccgcacctttgttcacccgaccagcaacaaacagttgtgctttatgcagcatattatcgaaacgctgcatcccggcggtcgtgcggcggtggtggtgccggataacgtgctgtttgaaggcggcaaaggcaccgacattcgtcgtgacctgatggataagtgtcatctgcacaccattctgcgtctgccgaccggtattttttacgctcagggcgtgaagaccaacgtgctgttctttaccaaagggacggtggcgaacccgaatcaggataagaactgtaccgatgatgtgtgggtgtatgacctgcgtaccaatatgccgagtttcggcaagcgcacaccgtttaccgacgagcatttgcagccgtttgagcgcgtgtatggcgaagacccgcacggtttaagcccgcgcactgaaggtgaatggagttttaacgccgaagagacggaagttgccgacagcgaagagaacaaaaacaccgaccagcatcttgctaccagccgctggcgcaagttcagccgtgagtggatccgcaccgcaaaatccgattcgctggatatctcctggctgaaagataaagacagtattgatgccgacagcctgccggagccggatgtattagcggcagaagcgatgggcgaactggtacaggcgctgtctgaactggatgcgctgatgcgtgaactgggggcgagcgatgaggccgatttgcagcgtcagttgctggaagaagcgtttggtggggtgaaggaatgagtgcggggaaattgccggaggggtgggttatcgccccagtatctacggtcacaactctaatccgaggagtaacgtataaaaaagagcaggcaataaattatctaaaagatgattatttgcctcttatccgtgcgaacaatattcagaatggcaagtttgatactacggacttggtttttgttcctaaaaatcttgttaaagaaagtcaaaaaatatctcctgaagatattgttattgcaatgtcatcagggagcaaatccgtagttggtaaatccgcacatcagcatctaccatttgaatgtagtttcggcgcattttgcggtgtattacgtcctgaaaaacttatattttctggttttattgctcatttcacaaaatcttctctttatcgaaacaaaatttcatcactttctgctggtgcaaatattaataatattaagccggcaagctttgatttgataaatataccaatcccaccacttgccgaacaaaaaatcatcgctgaaaaactcgatacgctgctggcgcaggtagacagcaccaaagcacgttttgagcaaatcccacaaatcctgaaacgttttcgtcaagcggtattggggggcgcagttaatggaaaattgacagaaaaatggcgtaattttgagccgcaacattctgtatttaagaagttaaattttgaatctatcttaactgaattacgtaatggtctttcatcaaagccaaatgaaagtggtgttggtcatccaatactacgcattagttctgtacgtgctggccatgtagatcaaaacgatattcggtttctagaatgttcagaaagtgaactaaaccgccacaaattacaagatggagatcttttatttactcgctataacggaagtttagaatttgttggtgtttgtgggttattgaaaaaattacaacatcaaaatttgctatatcctgataaacttattcgagctcgattaaccaaagatgctttaccagaatatatcgaaatatttttttcatccccctcagcacgaaatgcaatgatgaactgcgtgaaaacaacttctggtcaaaaaggtatttcaggaaaagatatcaaatcccaagttgttttattacctccagtaaaagaacaagccgaaatcgttcgccgcgtcgagcaactcttcgcctacgccgacaccatagaaaaacaggtcaacaacgccttagcccgcgtcaacaacctgacgcaatccatcctggcaaaagcgttccgtggtgaacttaccgcccagtggcgggccgaaaacccggatttgatcagcggagaaaacagcgccgccgcgttgctggaaaaaatcaaagctgaacgcgcagctagcgggggtaaaaaagcctcacgtaaaaaatcctgaacattattttctggcgcacctttccggtgcgctttttattatttcacgccaatcataacccacataaatatatttaaatcattccagaaattgcccattttattctatttttagctggactttccccatatttactgatgatatatacaggtatttagcgcggtgcggatgtgcgccaacacacccgcatcgctaatcacaatcactattcctggagaatagcagttatgactgacacgcattctattgcacaaccgttcgaagcagaagtctccccggcaaataaccgtcatgtcaccgtcggttatgcgagtcgctacccggattacagccgtattcccgccatcaccctgaaaggtcagtggctggaagccgccggttttgccactggcacggcggtagatgtcaaagtgatggaaggctgtattgtcctcaccgcccaaccacccgccgccgaggagagcgaactgatgcagtcgctacgccaggtgtgcaaactgtcggcgcgtaaacaaaagcaggtgcaggcgtttattggagtgattgccggtaaacagaaagtcgcgtaacagcgtttactcccggttaaccaccgggagccttccactgactcaatagaaactttccccctcagtaaatatttaccagtctgattttgcagtaaaaatctattgtttcagtacgttgcgaaagcgataatagaggcttagcaatgaggaaggcatatcttatggaatctattcaaccctggattgaaaaatttattaagcaagcacagcaacaacgttcgcaatccactaaagattatccaacgtcttaccgtaacctgcgagtaaaattgagtttcggttatggtaattttacgtctattccctggtttgcatttcttggagaaggtcaggaagcttctaacggtatatatcccgttattctctattataaagattttgatgagttggttttggcttatggtataagcgacacgaatgaaccacatgcccaatggcagttctcttcagacatacctaaaacaatcgcagagtattttcaggcaacttcgggtgtatatcctaaaaaatacggacagtcctattacgcctgttcccaaaaagtctcacagggtattgattacacccgatttgcctctatgctggacaacataatcaacgactataaattaatatttaattctggcaagagtgttattccacctatgtcaaaaactgaatcatactgtctggaagatgcgttaaatgatttgtttatccctgaaaccacaatagagacgatactcaaacgattaaccatcaaaaaaaatattatcctccaggggccgcccggcgttggaaaaacctttgttgcacgccgtctggcttacttgctgacaggagaaaaggctccgcaacgcgtcaatatggttcagttccatcaatcttatagctatgaggattttatacagggctatcgtccgaatggcgtcggcttccgacgtaaagacggcatattttacaatttttgtcagcaagctaaagagcagccagagaaaaagtatatttttattatagatgaaatcaatcgtgccaatctcagtaaagtatttggcgaagtgatgatgttaatggaacatgataaacgaggtgaaaactggtctgttcccctaacctactccgaaaacgatgaagaacgattctatgtcccggagaatgtttatatcatcggtttaatgaatactgccgatcgctctctggccgttgttgactatgccctacgcagacgattttctttcatagatattgagccaggttttgatacaccacagttccggaattttttactgaataaaaaagcagaaccttcatttgttgagtctttatgccaaaaaatgaacgagttgaaccaggaaatcagcaaagaggccactatccttgggaaaggattccgcattgggcatagttacttctgctgtgggttggaagatggcacctctccggatacgcaatggcttaatgaaattgtgatgacggatatcgcccctttactcgaagaatatttctttgatgacccctataaacaacagaaatggaccaacaaattattaggggactcatagtggaacagcccgtgatacctgtccgtaatatctattacatgcttacctatgcatggggttatttacaggaaattaagcaggcaaaccttgaagccatacccggtaacaatcttcttgatatcctggggtatgtattaaataaaggggttttacagctttcacgccgagggcttgagcttgattacaatcctaacaccgagatcattcctggcatcaaagggcgaatagagtttgctaaaacaatacgcggcttccatcttaatcatgggaaaaccgtcagtacttttgatatgcttaatgaagacacgctggctaaccgaattataaaaagcacattagccatattaattaagcatgaaaagttaaattcaactatcagagatgaagctcgttcactttatagaaaattaccgggcattagcactcttcatttaactccgcagcatttcagctatctgaatggcggaaaaaatacgcgttattataaattcgttatcagtgtctgcaaattcatcgtcaataattctattccaggtcaaaacaaaggacactaccgtttctatgattttgaaagaaacgaaaaagagatgtcattactttatcaaaagtttctttatgaattttgccgtcgtgaattaacgtctgcaaacacaacccgctcttatttaaaatgggatgcatcgagtatatcggatcagtcacttaatttgttacctcgaatggaaactgacatcaccattcgctcatcagaaaaaatacttatcgttgacgccaaatactataagagcattttttcacgacgaatgggaacagaaaaatttcattcgcaaaatctttatcaactgatgaattacttatggtcgttaaagcctgaaaatggcgaaaacataggggggttattaatatatccccacgtagataccgcagtgaaacatcgttataaaattaatggcttcgatattggcttgtgtaccgtcaatttaggtcaggaatggccgtgtatacatcaagaattactcgacattttcgatgaatatctcaaataaaatatcaggccggatgcggctgcgccttatccggcccataaccccttacttcctcaaccccgcaaacgcagcccgaatctcttcctccggcagctggatc

SEQ ID NO: 39 - DNA, 1262 bp, Artificial Sequence; identical to SEQ ID NO: 34
  Synthetic

SEQ ID NO: 40 - DNA, 1237 bp, Artificial Sequence
  Synthetic
agagttgggcaacggatgtgctggtggaggtgatcgcctcctgatgatgagccgctcccgatgtggtgtcgggagcggtattttctataaaacttaccgcttgacagctagctcagtcctaggtataatgctagctactagagaaagaggagaagatgccagacatgaagctgtttgcgggtaacgcgacccctgagctggcccagcgtatcgcgaaccgcttgtacacgagcctgggtgacgcagcggttggccgtttcagcgatggtgaagtcagcgtgcagattaatgaaaatgtgcgtggtggcgacattttcatcattcagagcacctgtgcgccgacgaacgataacctgatggaattggttgtgatggtcgatgcactgcgtcgcgcctccgccggtcgcattaccgcggtgattccgtattttggctatgcacgccaggatcgtcgtgtccgctccgcgcgcgtcccgatcacggcgaaagtcgtcgcggattttctgagcagcgtgggtgttgaccgtgtgctgaccgtggcgttgcatgctgagcaaattcaaggtttcttcgacgtcccggtggataatgttttcggttctccgattcttctggaagatatgctgcaactgaatctggataatccgatcgtcgttagcccggatatcggtggcgtggtgcgtgcgcgtgcaattgcaaagctgctgaatgataccgacatggcaatcatcgacaagcgccgtccgcgtgcgaatgtcagccaagtcatgcacatcattggcgacgttgctggccgtgactgcgttttagtggacgacatgatcgatacgggtggcactctgtgtaaagccgctgaggccctgaaagagcgcggtgcgaaacgtgttttcgcatacgcgacgcacccgatctttagcggtaatgctgcgaacaacttgcgtaactctgttattgacgaagttgttgtttgcgacaccattccgctgagcgacgaaatcaagagcctgccgaacgtgcgtaccctgaccctgagcggcatgctcgcagaggccatcagacgtattagcaacgaagagtcgatcagcgcgatgtttgagcattgacgcaaaaaaccccgcttcggcggggttttttcgcaatatcaggccggatgcggctgcgccttatccggcccataaccccttacttcctcaaccccgcaaacgcagcccgaatctcttcctccggcagctggatc

(19) United States
(12) Patent Application Publication
(10) Pub. No.: US 2023/0287437 A1
(43) Pub. Date: Sep. 14, 2023

(54) BACTERIAL STRAINS FOR DNA PRODUCTION

(71) Applicant: ModernaTX, Inc., Cambridge, MA (US)

(72) Inventors: Kevin Smith, Cambridge, MA (US); Marcus Duvall, Cambridge, MA (US); Bhargav Tilak, Cambridge, MA (US)

(73) Assignee: ModernaTX, Inc., Cambridge, MA (US)

(21) Appl. No.: 18/008,139

(22) PCT Filed: Jun. 3, 2021
(86) PCT No.: PCT/US2021/035636

§ 371 Date: Dec. 2, 2022

Related U.S. Application Data
(60) Provisional application No. 63/035,630, filed on Jun. 5, 2020.

(51) Int. Cl. C12N 15/70

(52) U.S. Cl. CPC C12N 15/70; C12N 2800/22; C12N 2800/101; C12N 2800/50; C12N 2820/005

(57) ABSTRACT

Compositions for the production of plasmid nucleic acids and methods of making and using the same are provided.

### CROSS-REFERENCE TO RELATED APPLICATIONS

This application claims the benefit under 35 U.S.C. § 119(e) of the filing date of U.S. Patent Application Ser. No. 63/035,630, filed Jun. 5, 2020, the contents which is hereby incorporated herein in its entirety by reference.

### BACKGROUND

Escherichia coli (E. coli) has a long history in biotechnology and drug development, and has been used as a host for plasmid DNA production for many years. This is due to a variety of reasons, among them are E. coli's genetic simplicity (e.g., smaller number of genes of ˜4,400), growth rate, safety, success in hosting foreign DNA, and ease of care. E. coli's long history and use have also made it a well characterized organism which has been manipulated in various ways. For example, several different strains have been constructed for different purposes including cloning, plasmid DNA production, and protein expression. Most commonly, E. coli K12 derivatives used such as DH5α, JM108, DH10β, and others are used for plasmid DNA cloning and production because they possess specific genomic mutations that are desirable for cloning purposes. These primarily result in the inactivation of genes that encode nucleases, recombinases, and other enzymes that reduce DNA stability, purity, and cloning efficiency of the strain.

### SUMMARY

Provided herein are engineered bacterial strains and vectors for enhanced plasmid DNA production.

In an aspect, the invention is an engineered nucleic acid vector comprising a stationary-phase-induced promoter and a primosome assembly site (PAS). In some embodiments the vector further includes point-mutations causing the formation of a critical stem-loop on RNAII, SL4. In some embodiments a native promoter for RNAII has been disrupted. In some embodiments a native promoter for RNAII has been deleted.

In some embodiments the stationary-phase-induced promoter is P(osmY). In some embodiments the P(osmY) has a sequence of SEQ ID NO: 27. In some embodiments the PAS has a sequence of SEQ ID NO: 28.

In some embodiments the SL4 has a sequence of SEQ ID NO: 29. In some embodiments the vector is Plasmid 1 (+PAS+P(osmY)).

In some embodiments the vector is Plasmid 2 (+PAS+P(osmY)+SL4). In some embodiments the vector has a sequence of at least 70% sequence identity to SEQ ID NO: 19 (sequence of Plasmid 1). In some embodiments the vector has a sequence of at least 70% sequence identity to SEQ ID NO: 20 (sequence of Plasmid 2).

In some embodiments the vector further comprises in the following 5′ to 3′ configuration:
(a) an origin of replication;
(b) the promoter; and
(c) an antibiotic resistance gene.

In some embodiments the vector further comprises an open reading frame (ORF) encoding an mRNA of interest.

In other aspects, a recombinant plasmid comprising the geneotype:|<repAlori_ts|<recA|<bla|<tetR|<P(tetR)>|gamma>|beta>|exo>|a>| is provided.

In other aspects, a recombinant plasmid comprising a nucleic acid sequence with at least 70% identity to SEQ ID NO: 19 is provided.

In other aspects, a recombinant plasmid comprising a nucleic acid sequence with at least 70% identity to SEQ ID NO: 20 is provided.

A method of performing an in vitro transcription reaction is provided in other aspects of the invention, the method using the engineered nucleic acid vector as described herein.

In some aspects the invention is a nucleic acid comprising a prsA variant. In some embodiments the nucleic acid has 70%-99% sequence identity to prsA. In some embodiments the nucleic acid has at least 70% sequence identity to prsA* (SEQ ID NO: 23). In some embodiments the nucleic acid has at least 80% sequence identity to prsA* (SEQ ID NO: 23). In some embodiments the nucleic acid has at least 90%, 95% or 100% sequence identity to prsA* (SEQ ID NO: 23). In some embodiments the nucleic acid is SEQ ID NO: 23. In some embodiments the nucleic acid encodes a protein having at least 95% sequence identity to prsA*

(SEQ ID NO: 24). In other embodiments the nucleic acid has 100% sequence identity to SEQ ID NO: 23 or encodes a protein having 100% sequence identity to SEQ ID NO: 24.

A genetically modified microorganism comprising a prsA variant, wherein the microorganism has a genome in which a repressor gene purR has been disrupted is provided in other aspects of the invention. In some embodiments the prsA variant has 70%-99% sequence identity to prsA. In some embodiments the prsA variant has least 90% sequence identity to prsA* (SEQ ID NO: 23). In some embodiments the prsA variant has SEQ ID NO: 23. In some embodiments the purR has been deleted. In some embodiments the purR has SEQ ID NO: 25. In some embodiments an EcoKI restriction system has been deleted from the genome. In some embodiments endA has been deleted from the genome. In some embodiments recA has been deleted from the genome. In some embodiments the genetically modified microorganism is a recombinant strain of Escherichia coli (E. coli).

In some aspects of the invention a recombinant strain of Escherichia coli (E. coli), comprising: an E. coli genome with at least the following gene deletions: endA (ΔendA) and recA (ΔrecA) is provided. In some embodiments the E. coli is derived from MG1655. In some embodiments the E. coli genome comprises a nucleic acid sequence of MG1655 genome including at least the following gene deletions: endA (ΔendA) and recA (ΔrecA) with respect to the MG1655 genome. In some embodiments the E. coli genome comprises a nucleic acid sequence of at least 95% sequence identity with MG1655 genome. In some embodiments an EcoKI restriction system has been deleted from the genome of the E. coli.

In some embodiments the E. coli genome comprises a nucleic acid sequence with at least 80% identity to MG1655 genome. In some embodiments the the E. coli genome comprises a nucleic acid sequence of wherein the E. coli genome comprises a nucleic acid sequence of MG1655 genome including the EcoKI restriction system deletion with respect to the MG1655 genome. In some embodiments the E. coli comprises a prsA variant. In some embodiments the wherein the E. coli genome comprises a nucleic acid sequence with at least 80% identity to MG1655 genome. In some embodiments the E. coli genome comprises a nucleic acid sequence of SEQ ID NO: 23. In some embodiments a purR sequence has been deleted from the genome of the E. coli. In some embodiments the E. coli genome comprises a nucleic acid sequence with at least 80% identity to MG1655 genome. In some embodiments the E. coli genome has a nucleic acid sequence of SEQ ID NO: 25 deleted with respect to the MG1655 genome.

In an aspect, the disclosure relates to a recombinant strain of E. coli, comprising an E. coli genome with at least the following gene deletions: endA and recA.

In some embodiments, the E. coli genome further comprises at least one of gene deletions selected from the group comprising: mrr; hsdR; hsdM; hsdS; symE; and mcrBC.

In some embodiments, the E. coli genome further comprises at least two of gene deletions selected from the group comprising: mrr; hsdR; hsdM; hsdS; symE; and mcrBC. In some embodiments, the E. coli genome further comprises at least three of gene deletions selected from the group comprising: mrr; hsdR; hsdM; hsdS; symE; and mcrBC. In some embodiments, the E. coli genome further comprises at least four of gene deletions selected from the group comprising: mrr; hsdR; hsdM; hsdS; symE; and mcrBC. In some embodiments, the E. coli genome further comprises at least five of gene deletions selected from the group comprising: mrr; hsdR; hsdM; hsdS; symE; and mcrBC. In some embodiments, the E. coli genome further comprises the gene deletions: mrr; hsdR; hsdM; hsdS; symE; and mcrBC.

In some embodiments, the E. coli genome is derived from the E. coli strain MG1655 or Strain 1. In some embodiments, the E. coli genome is derived from the E. coli Strain 4.

In an aspect, the disclosure relates to a recombinant strain of E. coli, wherein the E. coli genome further comprises a plasmid. In some embodiments, the plasmid expresses prsA* or is capable of knocking-out purR. In some embodiments, the plasmid both expresses prsA* and is capable of knocking-out purR.

In an aspect, the disclosure relates to a recombinant plasmid comprising the genotype:

|repA101|ori101_ts|<recA|<bla|<tetR|<P(tetR)|P(tet)>|gamma>|beta>|exo>|160a>|.

In some embodiments, the E. coli genomes disclosed herein may further express a gene for a positive selection marker based on a first environmental factor or a negative selection maker based on a second environmental factor, wherein the first and second environmental factors are not the same. In some embodiments, the E. coli genomes disclosed herein may further express a gene for a positive selection marker based on a first environmental factor and a negative selection maker based on a second environmental factor, wherein the first and second environmental factors are not the same. In some embodiments, the positive selection marker is a gene capable of conferring kanamycin resistance. In some embodiments, the negative selection marker is capable of expressing levansucrase.

In some aspects a genetically modified microorganism comprising Strain 3 is provided.

In some aspects a genetically modified microorganism comprising Strain 4 is provided.

In some aspects an engineered nucleic acid vector comprising a nucleic acid having at least 70% sequence identity to SEQ ID NO: 21 is provided.

In some aspects an engineered nucleic acid vector comprising a nucleic acid having at least 80% sequence identity to SEQ ID NO: 21 is provided.

In some aspects an engineered nucleic acid vector comprising a nucleic acid having at least 90% sequence identity to SEQ ID NO: 21 is provided.

In some aspects an engineered nucleic acid vector comprising a nucleic acid having at least 95% sequence identity to SEQ ID NO: 21 is provided.

In some aspects an engineered nucleic acid vector comprising a nucleic acid having SEQ ID NO: 21 is provided.

In some aspects an engineered nucleic acid vector comprising a nucleic acid having at least 70% sequence identity to SEQ ID NO: 22 is provided.

In some aspects an engineered nucleic acid vector comprising a nucleic acid having at least 80% sequence identity to SEQ ID NO: 22 is provided.

In some aspects an engineered nucleic acid vector comprising a nucleic acid having at least 90% sequence identity to SEQ ID NO: 22 is provided.

In some aspects an engineered nucleic acid vector comprising a nucleic acid having at least 95% sequence identity to SEQ ID NO: 22 is provided.

In some aspects an engineered nucleic acid vector comprising a nucleic acid having SEQ ID NO: 22 is provided.

In some aspects, an engineered nucleic acid vector comprising a nucleic acid sequence having at least 70% sequence identity to any one of SEQ ID NO: 1-15 is provided.

In some aspects, an engineered nucleic acid vector comprising a nucleic acid sequence having at least 80% sequence identity to any one of SEQ ID NO: 1-15 is provided.

In some aspects, an engineered nucleic acid vector comprising a nucleic acid sequence having at least 90% sequence identity to any one of SEQ ID NO: 1-15 is provided.

In some aspects, an engineered nucleic acid vector comprising a nucleic acid sequence having at least 95% sequence identity to any one of SEQ ID NO: 1-15 is provided.

In some aspects, an engineered nucleic acid vector comprising a nucleic acid sequence having at least 99% sequence identity to any one of SEQ ID NO: 1-15 is provided.

In some aspects, an engineered nucleic acid vector comprising a nucleic acid sequence of any one of SEQ ID NO: 1-15 is provided.

In some aspects, an engineered nucleic acid vector comprising a nucleic acid sequence having at least 70% sequence identity to SEQ ID NO: 10 is provided.

In some aspects, an engineered nucleic acid vector comprising a nucleic acid sequence having at least 70% sequence identity to SEQ ID NO: 11 is provided.

In some aspects, an engineered nucleic acid vector comprising a nucleic acid sequence having at least 95% sequence identity to SEQ ID NO: 10 is provided.

In some aspects, an engineered nucleic acid vector comprising a nucleic acid sequence having at least 95% sequence identity to SEQ ID NO: 11 is provided.

In some aspects, an engineered nucleic acid vector comprising a nucleic acid sequence having at least 99% sequence identity to SEQ ID NO: 10 is provided.

In some aspects, an engineered nucleic acid vector comprising a nucleic acid sequence having at least 99% sequence identity to SEQ ID NO: 11 is provided.

In some aspects, an engineered nucleic acid vector comprising a nucleic acid sequence of SEQ ID NO: 10 is provided.

In some aspects, an engineered nucleic acid vector comprising a nucleic acid sequence of SEQ ID NO: 11 is provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B show a representation of purine and pyrimidine biosynthesis in wild type E. coli K12 strains (FIG. 1A) and a representation of increased carbon flux to purine synthesis from Strain 4 due to genomic-borne overexpression of PrsA* and a purR knockout (FIG. 1B).

FIG. 2 shows exemplary positive and negative selection strategies used to introduce gene knockouts into E. coli.

FIG. 3 shows the lineage of Strain 2, Strain 3, and Strain 4 to their parental strain, Strain 1.

FIG. 4 is a graph depicting the percent supercoiled monomer of various plasmids prepped from Strain 1.

FIGS. 5A-5C show plasmid yields (FIG. 5A), culture densities (FIG. 5B), and Ct differential values (FIG. 5C) obtained from shake flask cultures containing Strain 1/Plasmid 1 (SEQ ID NO: 19) and single-copy plasmids carrying the gene designated on y-axis.

FIG. 6 shows plasmid copy number of PL-007948 in Strain 1 harboring single-copy plasmid for expression of prsA*.

FIGS. 7A-7B show plasmid yields (FIG. 7A) and culture densities (FIG. 7B) of Strain 3 and Strain 1 harboring Plasmid 1 (SEQ ID NO: 19) at 16 hours in shake flasks.

FIGS. 8A-8B show optical densities (FIG. 8A) and plasmid DNA yields (FIG. 8B) obtained from Strain 1, Strain 3, and Strain 4 harboring PL-007948.

FIGS. 9A-9B show plasmid DNA (pDNA) produced by Strain 3 and Strain 1 in Ambr250 system. FIG. 9A shows a kinetic profile of pDNA accumulation. FIG. 9B shows statistical analyses of pDNA produced by Strain 3 and Strain 1 at 22-hour EFT.

FIG. 10 shows specific productivity of Strain 3 and Strain 1 over time.

FIGS. 11A-11 show pDNA production with Strain 4 and Strain 1 in Ambr250 system. FIG. 11A shows a kinetic profile of pDNA accumulation. FIG. 11B shows statistical analyses of pDNA produced by Strain 1 and Strain 4 at 22-hour EFT.

FIG. 12 shows specific productivities of Strain 1 and Strain 4 over time.

FIGS. 13A-13C depict a process diagram of a long-term pDNA stability experiment. The figure show: strains harboring two different plasmids were grown up and passaged into fresh media for several days (FIG. 13A) followed by poly-A tail sanger sequencing; total number of generations of NEB strain (strain similar to commercially available strains), Strain 1 and Strain 4 harboring the indicated plasmids (FIG. 13B); and a process flow diagram modeling the number (#) of generations a strain is expected to undergo from MCB vial to the end of a 30 or 300-liter scale fermentation scale. ‘MCB’—master cell bank, ‘WCB’—working cell bank (FIG. 13C).

FIG. 14 shows growth profiles of Strain 1 and Strain 4 harboring indicated plasmids.

FIG. 15 shows a graph depicting plasmid DNA production over time in strains Strain 1 and Strain 4 with Plasmid 1.

FIG. 16 shows a plasmid map with modifications made to construct Plasmid 1 and Plasmid 2.

FIGS. 17A-17B show plasmid yields obtained in Strain 1 using various plasmids (FIG. 17A) and final culture optical densities at 16 hours (FIG. 17B).

FIGS. 18A-18B show plasmid production data for modification 9 (SEQ ID NO: 10; Ori10) and modification 10 (SEQ ID NO: 11; Ori11). FIG. 18A shows milligrams per liter (mg/liter) plasmid DNA (pDNA) increase over the parent plasmid (SEQ ID NO: 16); based on control plasmids (PL_007984). FIG. 18B shows improved overall productivity as measured by mg of pDNA per gram of wet cell weight (gWCW).

### DETAILED DESCRIPTION

E. coli has been used as a host for plasmid DNA production for many years. Several different strains have been constructed for many different purposes including cloning, plasmid DNA production, and protein expression. Most commonly, E. coli K12 derivatives such as DH5α, JM108, DH10β and others are used for plasmid DNA cloning and production. These primarily result in the inactivation of genes that encode nucleases, recombinases, and other enzymes that reduce DNA stability, purity, and cloning efficiency of the strain.

Additionally, E. coli, among other organisms, possess regulatory pathways which limit or modulate expression of other products, which may be desirable to have in larger quantities (e.g., nucleotides). Thus, while the genes controlling these pathways are active, it is difficult to increase the efficiency of the E. coli in producing a desired product.

Provided are a number of developments in strain and vector engineering that provide significant improvements in plasmid DNA production. The improvements include the identification and manipulation of an enzyme (PrsA) in E. coli that, when overexpressed, results in higher plasmid DNA yield. Variants of this enzyme that significantly disrupt feedback-inhibition by downstream metabolites have been developed and incorporated into host cells. In the host, the variant enzymes can mobilize carbon through the DNA biosynthesis pathways of cell. Other engineering developments include the knock-out of the repressor, PurR, of the DNA synthesis pathway from the genome. E coli strains incorporating the engineered improvements described herein have significantly enhanced yields, e.g., greater than 2 times increase in plasmid yield have been observed, a quite significant improvement.

While E. coli has been used in a variety of ways, it still has limitations regarding its use for particular applications, and in instances can leave much to be desired. The improved strains described herein provide significant advantages over prior art strains. The strains disclosed herein involve various combinations of engineered components, including, for instance, a deleted or mutated EcoKI restriction system, an endA deletion (ΔendA, endonuclease that can degrade plasmid DNA during purification), a recA deletion (ΔrecA, recombinase that is a contributor to DNA and poly-A tail instability), addition of a PrsA enzyme, deletion of purR (encodes transcriptional repressor of the nucleotide biosynthesis pathway), and/or deletion of one or more of mrr; hsdR; hsdM; hsdS; symE; and mcrBC.

Also provided are enhanced methods for plasmid DNA production as well as tools and compositions involved in those methods. A first-generation custom E. coli strain, referred to as Strain 1, contains two gene deletions: ΔendA (an endonuclease that can degrade plasmid DNA during purification) and ΔrecA (a recombinase that is a major contributor to DNA and poly-A tail instability). This strain was further manipulated to remove the EcoKI restriction system in order to produce a new strain referred to herein as Strain 2.

Native E. coli possess the EcoKI restriction system. EcoKI is a restriction-modification enzyme complex responsible for identifying and restricting unmethylated, foreign DNA, and for modifying native, hemimethylated DNA by methylation for self-identification. Left alone, the EcoKI system will recognize non-methylated DNA as foreign and, if the DNA also possesses unique EcoKI-recognition sites, degrade it. While it is not essential to inactivate the EcoKI system from E. coli to clone plasmid DNA, deletion does significantly increase cloning and transformation efficiencies if the desired plasmid DNA possesses EcoKI recognition sites.

Thus, in some aspects, the disclosure relates to a recombinant strain of E. coli comprising an E. coli genome with at least the following gene deletions: endA and recA. The endA gene encodes endonuclease-1 protein, which when expressed can induce double-strand break activity. This activity can degrade and otherwise compromise the production of plasmid DNA by E. coli possessing the gene. The recA gene encodes the recA protein, which is relates to the repair and maintenance of DNA. However, recA through its properties in facilitating DNA repair, can play a role in the homologous recombination of DNA, as well as mediating homology pairing, homologous recombination, DNA break repair, and the SOS response, wherein DNA damage triggers the cell cycle to arrest initiate DNA repair and mutagenesis. The properties of both endA and recA are not beneficial in the production of consistent and identical DNA plasmids. In some embodiments, the recombinant strain of E. coli comprises an E. coli genome with deletions of endA and recA.

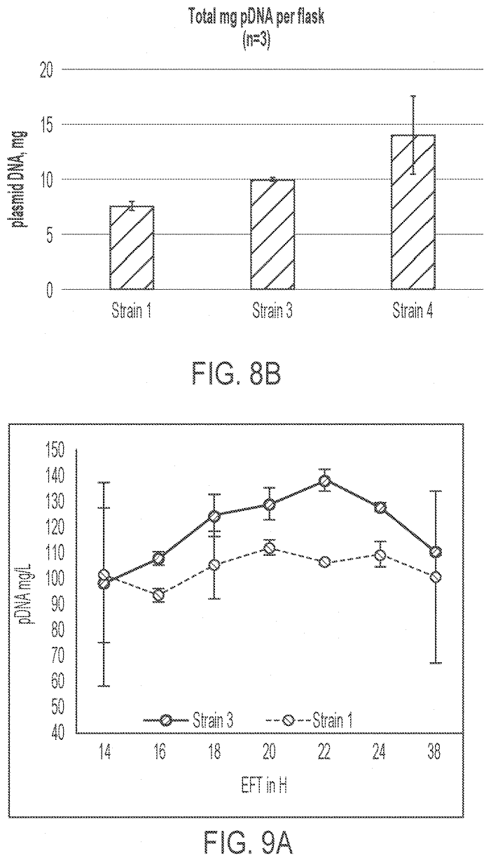

In some aspects, the disclosure relates to a recombinant strain of E. coli, wherein the E. coli genome further comprises an exogenous DNA encoding a purine biosynthetic enzyme. The exogenous DNA is integrated into the E. coli genome. Integration of a prsA*, encoding a mutant purine biosynthetic enzyme, expression cassette into the genome of Strain 2 or Strain 1 provides substantial enhancements to plasmid DNA yield. A strain designed from the Strain 2 and adding the prsA* is referred to as Strain 3. Strain 3 may be further modified by knocking out purR, which encodes a transcriptional repressor of the nucleotide biosynthesis pathway. This strain, referred to herein as Strain 4, can have further functional enhancements. When Strain 4 was tested, along with Strain 1 and Strain 3 for plasmid DNA productivity, each of Strain 1, Strain 3 and Strain 4 showed higher improved plasmid DNA yields over original E. coli strains (shown in FIG. 8A). Of the three tested strains, the Strain 1 produced lower yields than Strain 3, which produced lower yields than Strain 4. Poly-A tail stability was also found to be improved in Strain 4 post-transformation and over many generations of growth (for instance see Table 4, which shows Strain 4 had improved poly-A tail stability post-transformation compared to commercial strain (control) and Strain 1).

In some embodiments the invention encompasses an E. coli strain comprising a gene encoding phosphoribosyl pyrophosphate synthetase protein (prsA). In other embodiments the invention encompasses an E. coli strain comprising a gene encoding a phosphoribosyl pyrophosphate synthetase protein variant (prsA*). The E. coli strain may comprise a prsA variant. In some embodiments the E. coli strain may comprise a prsA and a prsA variant. PRPP (phosphoribosyl pyrophosphate) is a pentose phosphate formed from ribose 5-phosphate and one ATP by the enzyme phosphoribosyl pyrophosphate synthetase encoded by the gene prsA. The production of phosphoribosyl pyrophosphate synthetase is an early step in the biosynthesis of purine, pyrimidine, and nicotinamide nucleotides and in the biosynthesis of histidine and tryptophan.

A prsA variant refers to a nucleic acid encoding a variant of the enzyme phosphoribosyl pyrophosphate synthetase having at least one amino acid difference from naturally occurring the enzyme phosphoribosyl pyrophosphate synthetase. Preferably, the prsA variant is resistant to negative feedback regulation by downstream metabolites in the DNA biosynthesis pathway. The resistance to negative feedback regulation prevents the pathway from being shut down to conserve energy, thus leading to enhanced processing of nucleic acid synthesis.

In some embodiments the prsA variant has at least 70% sequence identity to prsA. In some embodiments the prsA variant comprises a sequence with at least 70% sequence identity to prsA. In some embodiments the prsA variant comprises a sequence with at least 70% sequence identity to prsA, but includes at least one nucleotide difference, i.e., a deletion, insertion, or replacement. In some embodiments a prsA variant comprises prsA* (SEQ ID NO: 23). In some embodiments the prsA variant is prsA* (SEQ ID NO: 23). prsA* is also referred to as prsA_D128A. In other embodiments the prsA variant comprises a nucleic acid sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to SEQ ID NO: 23.

The “percent identity,” “sequence identity,” “% identity,” or “% sequence identity” (as they may be interchangeably used herein) of two sequences (e.g., nucleic acid or amino acid) refers to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range.

Some embodiments encompass an E. coli strain comprising a genome lacking a functional repressor gene purR. The genetic modification of an E. coli strain to reduce the effects of a feedback inhibitor/repressor purR can be useful for further promoting plasmid DNA synthesis in the systems disclosed herein. In some embodiments the purR gene is disrupted in E. coli by causing a frame shift mutation or knocking out the gene. Disruption of gene function may be effectuated such that the normal encoding of a functional enzyme purR by the purR gene has been altered so that the production of the functional enzyme in a microorganism has been reduced or eliminated. Disruption may broadly include a gene deletion, as well as, but is not limited to gene modification (e.g., introduction of stop codons, frame shift mutations, introduction or removal of portions of the gene, introduction of a degradation signal) affecting mRNA transcription levels and/or stability, and altering the promoter or repressor upstream of the gene encoding the polypeptide. In some embodiments, a gene disruption is taken to mean any genetic modification to the DNA, mRNA encoded from the DNA, and/or the amino acid sequence that results in at least a 50 percent reduction of enzyme function of the encoded gene in the microorganism. In some embodiments, purR comprises wild-type purR. In some embodiments, purR comprises a sequence with at least 70% identity to wild-type purR. In some embodiments purR comprises a sequence with at least 70% identity to SEQ ID NO: 25. In some embodiments purR comprises a sequence of SEQ ID NO: 25. In some embodiments purR has a sequence of SEQ ID NO: 25.

Thus, in some aspects, an E. coli strain expresses a prsA variant such as prsA* and/or purR expression is disrupted. In some embodiments, the plasmid both expresses prsA* and is capable of knocking-out purR.

In some aspects, the disclosure relates to a recombinant plasmid comprising the genotype: |repA101|ori101_ts|<recA|<bla|<tetR|<P(tetR)|P(tet)>|gamma>|beta>|exo>|60a>|.

In some embodiments, the recombinant plasmid comprises a nucleic acid sequence with at least 70% identity to SEQ ID NO: 26. In some embodiments, the recombinant plasmid comprises a nucleic acid sequence of SEQ ID NO: 26.

In some aspects, the disclosure relates to a recombinant strain of E. coli comprising a plasmid, wherein the plasmid has the genotype |<repA101|ori101_ts|<recA|<bla|<tetR|<P(tetR)|P(tet)>|gamma>|beta>|exo>|60a>|; a nucleic acid with at least 70% identity to SEQ ID NO: 26. In an aspect, the disclosure relates to a recombinant strain of E. coli, wherein the plasmid is the plasmid comprises the genotype |<repA101|ori101_ts|<recA|<bla|<tetR|<P(tetR)|P(tet)>|gamma>|beta>|exo>|60a>|.

Strain 3 and Strain 4 both were found to display higher plasmid DNA yields in comparison with Strain 1. Strain 3 produced higher pDNA than Strain 4 after 16 hours EFT (elapsed fermentation time). The yield for Strain 3 was statistically higher than that for Strain 1 at a 95% confidence interval. The specific productivity of Strain 4, calculated as pDNA produced (mg/L) per gram biomass was found to be significantly higher than Strain 1.

In some embodiments, the E. coli genome further comprises at least one gene deletion selected from the group comprising: mrr; hsdR; hsdM; hsdS; symE; and mcrBC.

The mrr gene encodes a protein mrr involved in the recognition and modulation of foreign DNA, specifically to restrict (i.e., degrade) adenine- and cytosine-methylated DNA. The hsdR gene encodes Type I restriction enzyme EcoKI R protein which produces endonucleolytic cleavage of nucleic acids (e.g., DNA) to give random double-stranded fragments with terminal 5′-phosphates wherein ATP is simultaneously hydrolyzed. The hsdM gene encodes Type I restriction enzyme EcoKI M protein and the hsdS gene encodes Type I restriction enzyme EcoKI specificity (S) protein. The M and S subunits together form a methyltransferase (MTase) that methylates two adenine residues in complementary strands of a bipartite DNA recognition sequence. In the presence of the R subunit the complex can also act as an endonuclease, binding to the same target sequence but cutting the DNA some distance from this site. Whether the DNA is cut or modified depends on the methylation state of the target sequence. When the target site is unmodified, the DNA is cut. When the target site is hemimethylated, the complex acts as a maintenance MTase modifying the DNA so that both strands become methylated. (UniProt; www.uniprot.org/uniprot/P05719). The symE gene encodes toxic protein SymE, which is a protein involved in the degradation and recycling of damaged RNA. Overexpression of SymE protein may be toxic for the cell, affecting colony-forming ability and protein synthesis. The mcrBC gene encodes the 5-methylcytosine-specific restriction enzyme McrBC, subunit McrB which is an endonuclease which cleaves DNA containing 5-methylcytosine or 5-hydroxymethylcytosine on one or both strands. In some embodiments, the E. coli genome further comprises at least two of gene deletions selected from the group comprising: mrr; hsdR; hsdM; hsdS; symE; and mcrBC. In some embodiments, the E. coli genome further comprises at least three of gene deletions selected from the group comprising: mrr; hsdR; hsdM; hsdS; symE; and mcrBC. In some embodiments, the E. coli genome further comprises at least four of gene deletions selected from the group comprising: mrr; hsdR; hsdM; hsdS; symE; and mcrBC. In some embodiments, the E. coli genome further comprises at least five of gene deletions selected from the group comprising: mrr; hsdR; hsdM; hsdS; symE; and mcrBC. In some embodiments, the E. coli genome further comprises the gene deletions: mrr; hsdR; hsdM; hsdS; symE; and mcrBC.

In some embodiments, the E. coli genome is derived from the E. coli strain MG1655 or Strain 1. In some embodiments, the E. coli genome is derived from the E. coli Strain 4 (Strain 4>ΔendA ΔrecA Δmrr-mcn:P(J23119)>prsA* ΔpurR).

In some aspects, engineered nucleic acid vectors having unique structural and functional attributes for enhanced plasmid production are provided. The nucleic acid vectors described herein have been engineered and synthesized using a novel combination of elements. The resultant nucleic acid vectors having one or more of the design modifications were found to have significantly increased yield of supercoiled product.

Efforts in vector engineering for plasmid DNA production have largely been focused on increasing plasmid DNA copy number and plasmid supercoiling. It has been discovered herein that combinations of several modifications to plasmid structure result in significant and unexpected enhancements in plasmid DNA yield and quality. The modifications include combinations of replacing the native promoter for RNAII (the primer for replication) with a stationary-phase-induced promoter, introducing point-mutations causing the formation of a critical stem-loop on RNAII, SL4, that is needed for plasmid DNA replication to begin, and/or incorporating a primosome assembly site on the plasmid backbone.

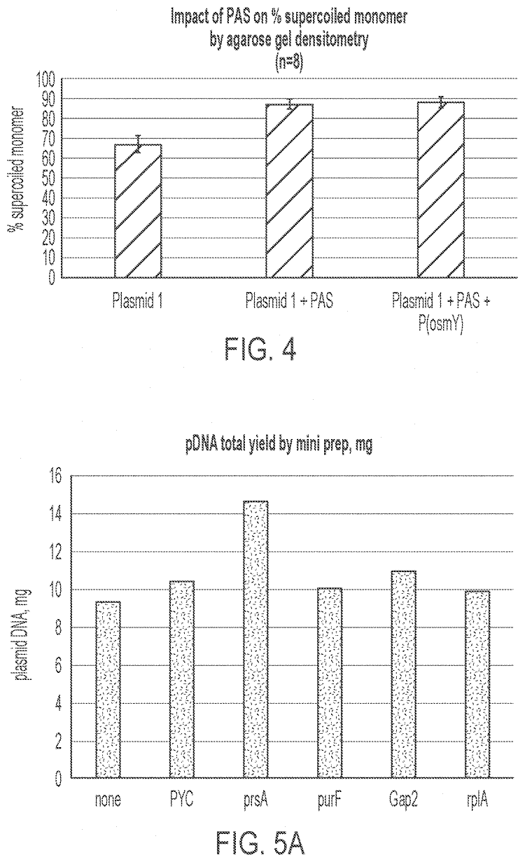

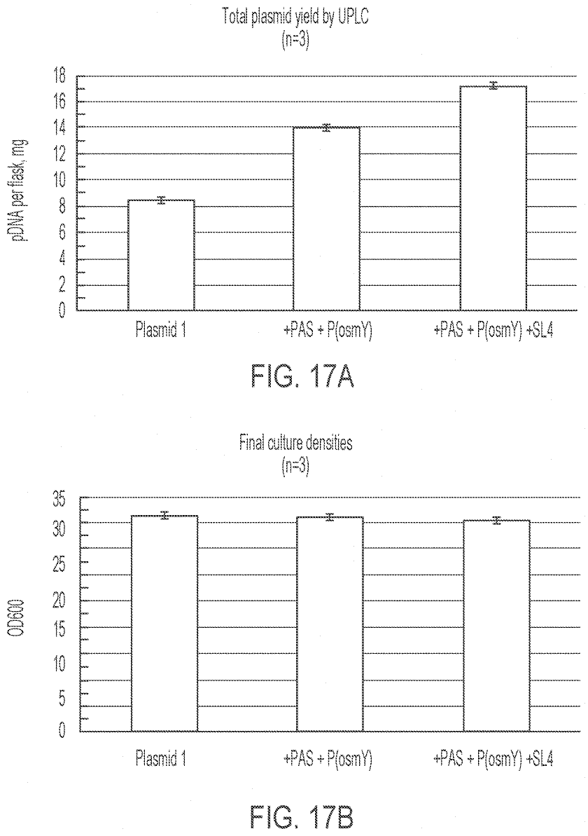

In some embodiments new enhanced plasmids were generated using these modifications to the plasmid's origin of replication (such as the plasmid shown in FIG. 16). Exemplary modified plasmids include: Plasmid 1 (+PAS+P(osmY)) and Plasmid 2 (+PAS+P(osmY)+SL4). The Plasmid 1 includes the native promoter for RNAII (the primer for replication) having been replaced with stationary-phase-induced promoter, P(osmY) and a primosome assembly site (PAS) inserted on the backbone. Plasmid 2 includes the modifications of Plasmid 1 and further adds the introduction of four point-mutations that encourage the formation of a critical stem-loop on RNAII, SL4, that is needed for pDNA replication to begin. These plasmids were tested in a variety of assays and plasmid DNA yields obtained with Plasmid 1 and Plasmid 2 were found to be significantly higher relative to the control plasmid, Plasmid 1 (SEQ ID NO: 19) (FIG. 17A and 17B). In addition, the introduction of PAS was shown to significantly increase the percentage of plasmid DNA that is supercoiled monomer (FIG. 4).

The RNAII promoter initiates plasmid DNA replication. The copy number can be controlled by relative ratios of RNAII (the primer) and RNAI (the inhibitor). It was determined that fine-tuning the strength and timing of RNAII expression could reduce overburdening E. coli, and thus increasing the plasmid yields. The RNAII promoter was targeted for various changes to increase RNAII expression by point mutation and through the addition of promoters for RNAII expression. In an attempt to completely remove the RNAII promoter and replace with E. coli promoters that are upregulated at stationary phase many were found to be toxic and strains were not viable. In strong contrast, replacement of native RNAII promoter in E. coli with P(osmY) promoter, a stationary-phase promoter, resulted in significant improvements. The ratio of osmY transcripts were about 50-fold higher at stationary-phase relative to log phase.

In some aspects the invention is a plasmid comprising a functional P(osmY) promoter. In some embodiments the plasmid does not have a functional RNAII promoter. A functional P(osmY) promoter can include a sequence having at least 70% sequence identity to SEQ ID NO: 27. In some embodiments the P(osmY) promoter is SEQ ID NO: 27. In other embodiments the P(osmY) promoter comprises a nucleic acid sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to SEQ ID NO: 27.

Additionally, Stem Loop 4 (SL4) mutations have been made to discourage RNAI inhibition. SL4 mutations can increase rate of SL4 formation, thus increasing replication rate.

The presence of a poly-A tail significantly impacts plasmid supercoiling and isomer distributions. It was found that the loss of supercoiling could be offset with the incorporation of PAS into the plasmid. The addition of PAS significantly increased the percent of supercoiled monomer, with modest yield improvement.

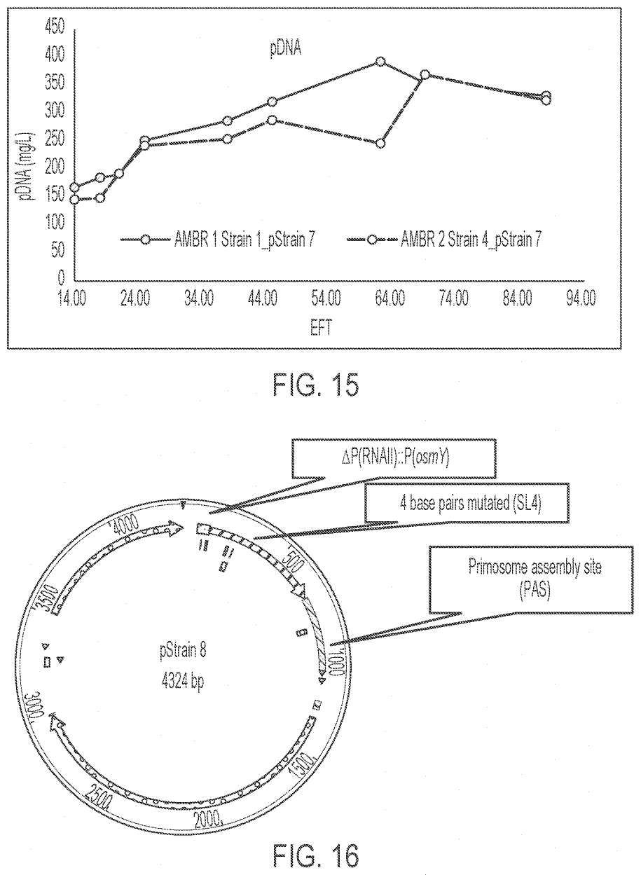

In addition to evaluating the novel strains disclosed herein with existing vector backbones, two strains, Strain 1 and Strain 4, were analyzed with an optimal engineered vector, Plasmid 1. With the Plasmid 1 vector, both Strain 1 and Strain 4 strains produced comparable amounts of plasmid DNA, which was two times higher than the plasmid DNA produced by the base vectors (FIG. 15).

A “nucleic acid” is at least two nucleotides covalently linked together, and in some instances, may contain phosphodiester bonds (e.g., a phosphodiester “backbone”). As used herein, the terms “nucleic acid sequence” and “polynucleotide” are used interchangeably and do not imply any length restriction. As used herein, the terms “nucleic acid” and “nucleotide” are used interchangeably. The terms “nucleic acid sequence” and “polynucleotide” embrace DNA (including cDNA) and RNA sequences. The nucleic acid sequences of the present invention include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

Engineered nucleic acids of the present disclosure may be produced using molecular biology methods. In some embodiments, engineered nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

The nucleic acid vectors of the invention also may have one or more terminator sequences present or removed. A terminator sequence is a nucleic acid sequence that signals the end of the expression cassette or transcribed region. Effective transcription vectors typically include one or more terminator sequences. Terminator sequences include, for instance, T7 and

T4 terminator sequences.

The preferred vectors of the invention may also have one or more resistant markers, or a marker that is unique to the particular vector. For instance, the vector may have originally had an ampicillin resistant marker. In some preferred embodiments of the invention the ampicillin marker is replaced with a different marker such as kanamycin resistant marker. In some embodiments, the E. coli genomes disclosed herein may further express a gene for a positive selection marker based on a first environmental factor or a negative selection maker based on a second environmental factor, wherein the first and second environmental factors are not the same. In some embodiments, the E. coli genomes disclosed herein may further express a gene for a positive selection marker based on a first environmental factor and a negative selection maker based on a second environmental factor, wherein the first and second environmental factors are not the same. In some embodiments, the positive selection marker is a gene capable of conferring kanamycin resistance. In some embodiments, the negative selection marker is capable of expressing levansucrase.

A vector disclosed herein may also have any pathogen derived sequences removed. Removal of pathogen derived sequences can have a positive effect on the product yield.

The origin of replication (ori) can be included in the nucleic acid and may be modified as disclosed herein. The nucleic acid may in some embodiments contain several ori, for example 2 ori's. It can, for example, be a combination of a low-copy ori and a temperature-dependent ori or for example ori's that allow propagation in various host organisms.

In some embodiments, a plasmid comprises an engineered nucleic acid vector. In some embodiments, a plasmid is replicated. In some embodiments, a plasmid comprises Plasmid 1 (SEQ ID NO: 19). In some embodiments, a plasmid comprises a sequence with at least 70% identity to SEQ ID NO: 19.

In some embodiments, a plasmid comprises an origin of replication (ori). In some embodiments, a plasmid comprises an ori comprising a sequence with at least 70% identity to SEQ ID NO: 16. In some embodiments, a plasmid comprises an ori comprising a sequence of SEQ ID NO: 16. In some embodiments, an ori comprises at least one mutation. In some embodiments, an ori mutation comprises at least one of the following: Ori1-Ori16. In some embodiments, an ori comprises a sequence with at least 70% identity to any one of SEQ ID NO: 1-15. In some embodiments, an ori comprises a sequence with at least 70% identity to SEQ ID NO: 10. In some embodiments, an ori comprises a sequence with at least 70% identity to SEQ ID NO: 11. In some embodiments, an ori comprises a sequence of any one of SEQ ID NO: 1-15. In some embodiments, an ori comprises a sequence of SEQ ID NO: 10. In some embodiments, an ori comprises a sequence of SEQ ID NO: 11.

The nucleic acids may also contain one or more elements from other vectors. For example other vectors include phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In other embodiments the nucleic acids described herein do not include any elements from any one or more of the other vectors.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

Thus, in some embodiments the nucleic acid vector has a nucleic acid sequence of SEQ ID NO: 21. In other embodiments the nucleic acid vector of the invention has a nucleic acid sequence having at least 70%, 75%, 80%, 82%, 84%, 85%, 86%, 88%, 90%, 92%, 94%, 95%, 96%, 98%, or 99% sequence identity to SEQ ID NO: 22.

A nucleic acid sequence or fragment thereof is “substantially homologous” or “substantially identical” to a reference sequence if, when optimally aligned (with appropriate nucleotide insertions or deletions) with the other nucleic acid (or its complementary strand), there is nucleotide sequence identity in at least about 70%, 75%, 80%, 82%, 84%, 85%, 86%, 88%, 90%, 92%, 94%, 95%, 96%, 98% or 99% of the nucleotide bases. Methods for sequence identity determination of nucleic acid sequences are known in the art.

A “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters may be more important than any single parameter.

There are many algorithms available to align two nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of nucleic acid sequences for comparison may be conducted, for example, by computer implemented algorithms (e.g. GAP, BESTFIT, FASTA or TFASTA), or BLAST and BLAST 2.0 algorithms.

In a sequence identity comparison, the identity may exist over a region of the sequences that is at least 10 nucleic acid residues in length, e.g. at least 15, 20, 30, 40, 50, 75, 100, 150, 200, 250, 300, 350, 400, 450, 500, 550, 600, 650, or 685 nucleotides in length, e.g. up to the entire length of the reference sequence.

Substantially homologous or substantially identical nucleic acids have one or more nucleotide substitutions, deletions, or additions. In many embodiments, those changes are of a minor nature, for example, involving only conservative nucleic acid substitutions that may result in the same amino acid being coded for during translation or in a different but conservative amino acid substitution. Conservative amino acid substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine. Substantially homologous nucleic acids also encompass those comprising other substitutions that do not significantly affect the folding or activity of a translation product.

The nucleic acid vector of the invention may be an empty vector or it may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide. An expression cassette encodes an RNA including at least the following elements: a 5′ untranslated region, an open reading frame region encoding the mRNA, a 3′ untranslated region and a polyA tail. The open reading frame may encode any mRNA.

A “5′ untranslated region (UTR)” refers to a region of an mRNA that is directly upstream (i.e., 5′) from the start codon (i.e., the first codon of an mRNA transcript translated by a ribosome) that does not encode a protein or peptide.

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

One of ordinary skill in the art appreciates that different species exhibit “preferential codon usage”. As used herein, the term “preferential codon usage” refers to codons that are most frequently used in cells of a certain species, thus favoring one or a few representatives of the possible codons encoding each amino acid. For example, the amino acid threonine (Thr) may be encoded by ACA, ACC, ACG, or ACT, but in mammalian host cells ACC is the most commonly used codon; in other species, different Thr codons may be preferential. Preferential codons for a particular host cell species can be introduced into the polynucleotides of the present invention by a variety of methods known in the art. Alternatively non-preferred codons may be used. In some embodiments of the invention, the nucleic acid sequence is codon optimized.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of the polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide).

A “nucleic acid vector” is a polynucleotide that carries at least one foreign or heterologous nucleic acid fragment. A nucleic acid vector may function like a “molecular carrier”, delivering fragments of nucleic acids respectively polynucleotides into a host cell or as a template for IVT. An “in vitro transcription (IVT) template,” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

In some embodiments the nucleic acid vector according to the invention is a circular nucleic acid such as a plasmid. In other embodiments it is a linearized nucleic acid. According to one embodiment the nucleic acid vector comprises a predefined restriction site, which can be used for linearization of the vector. Intelligent placement of the linearization restriction site is important, because the restriction site determines where the vector nucleic acid is opened/linearized. The restriction enzymes chosen for linearization should preferably not cut within the critical components of the vector.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right hand side, when following this convention.

### EXAMPLES

### Example 1: Host Strain Modifications Alter Plasmid Production

### Introduction

### Purpose and Significance

E. coli is a microorganism that has been used for cloning purposes and plasmid DNA production. A strain that also produces plasmid at high-yield, especially at large scale, would be valuable. Methods for increasing the plasmid DNA yield of E. coli using various metabolic engineering techniques are disclosed herein. In some instances, an endogenous DNA restriction system, EcoKI, was removed, which resulted in improved cloning efficiency of unmethylated plasmids.

Current Commercially Available Strains of E. coli for Cloning Plasmid DNA

E. coli K12 derivatives used such as DH5α, JM108, DH10β and others have been used for plasmid DNA cloning and production. These primarily result in the inactivation of genes that encode nucleases, recombinases and other enzymes that reduce DNA stability, purity and cloning efficiency of the strain. Here it is shown inactivation of all or part of the EcoKI restriction system to allow for the cloning of eukaryotic or non-methylated DNA. Left alone, the EcoKI system will recognize non-methylated DNA as foreign and, if the DNA also possesses unique EcoKI-recognition sites, degrade it. While it is not essential to inactivate the EcoKI system from E. coli to clone plasmid DNA, it does significantly increase cloning and transformation efficiencies if the desired plasmid DNA possesses EcoKI recognition sites (Table 1).

TABLE 1
Transformation efficiencies obtained with Strain 1 and various plasmids
containing different numbers of EcoKI restriction sites. ‘CFU’ denotes
colony forming units.

| Transformation Efficiency | | |
|---|---|---|
| (CFU/ug) | CFU (100 uL) | #EcoKI sites |
| 4.2 × 10⁵ | 750 | 0 |
| 5.2 × 10⁴ | 101 | 1 |
| 4.0 × 10² | 1 | 2 |

Nucleotide Biosynthesis in E. coli to Increase Flux Through Pathways

Nucleotides biosynthesis is a carbon, energy and redox-intensive process and, therefore, expression of the cell's nucleotide biosynthesis pathways is tightly controlled by transcriptional repression and, in addition, several key enzymes in these pathways are allosterically regulated by downstream metabolites and/or cofactors that are indicative of a low-energy state for the cell. Briefly, pyrimidine and purines are produced using a 5-carbon precursor, 5-phospho-α-D-ribose 1-diphosphate (PRPP), that serves as the primary building block for nucleotides. This metabolite is produced from D-ribose 5-phosphate (R5P), an intermediate in the pentose phosphate pathway, by ribose-phosphate diphosphokinase (PrsA). Because synthesizing PRPP commits carbon to the energy-intensive nucleotide biosynthesis pathways, the cell tightly regulates this step by controlling expression of the prsA gene and by modulating the activity of the PrsA enzyme by allosteric inhibition by ADP. E. coli also possesses a key transcriptional regulator of the pyrimidine and purine biosynthesis pathways, PurR, which is itself regulated by products of the purine pathway: inosine and guanine. When intracellular concentrations of inosine and/or guanine increase, the metabolites associate with the PurR enzyme and induce binding of PurR to promoters of its regulon of 32 genes to repress expression of the nucleotide biosynthesis pathways. Indeed, when the purR gene is knocked-out of the E. coli genome, genes that are normally repressed by PurR experience a significant increase in transcription. There are no known examples of metabolic engineering E. coli's nucleotide biosynthesis pathways for improving plasmid DNA productivity.

In this work, an E. coli strain, Strain 1 was created, and then a descendant of Strain 1 was created that had even further improved plasmid DNA yields (mg pDNA/mg biomass or plasmid copy number) and higher cloning efficiency by upregulating the activity of the purine and pyrimidine biosynthesis pathways and by removing the EcoKI restriction system, respectively.

### Methods

TABLE 2
Strains used

| | Plasmid | |
|---|---|---|
| | (SEQ | |
| Strain | ID NO) | |
| 1 | None | ΔendA ΔrecA |
| — | 26 | ΔendA ΔrecA |
| 5 | 26 | ΔendA ΔrecA Δ(mrr-hsdRMS-symE-mcrBC)::kan-sacB |
| 2 | None | ΔendA ΔrecA Δ(mrr-hsdRMS-symE-mcrBC) |
| 3 | None | ΔendA ΔrecA Δ(mrr-hsdRMS-symE-mcrBC)::P(J23119)-prsA_D128A |
| 6 | 26 | ΔendA ΔrecA Δ(mrr-hsdRMS-symE-mcrBC)::P(J23119)-prsA_D128A ΔpurR::kan-sacB |
| 4 | 26 | ΔendA ΔrecA Δ(mrr-hsdRMS-symE-mcrBC)::P(J23119)-prsA_D128A ΔpurR |

TABLE 3
Plasmids used

| | |
|---|---|
| Plasmid | |
| Plasmid 7 | \|<repA101\|ori101_ts\|<recA\|<bla\|<tetR\|<P(tetR)\|P(tet)>\|gamma>\|beta>\|exo>\|60a>= |
| SEQ ID NO: 26 | |
| Plasmid 1 | \|pUC ori\|P(T7)>\|Luc>\|T100\|Xbal site\|T7 terminator\|P(kan)>\|kanR\| |
| SEQ ID NO: 19 | |
| Plasmid 2 | \|pUC ori\|Xbal site\|P(T7)>\|EPO>\|T100\|Xbal site\|T7 terminator\|P(kan)>\|kanR\| |
| SEQ ID NO: 20 | |

Assaying Plasmid Yields of E. coli Strains in Shake Flasks

Each strain of E. coli was transformed with plasmids as specified in the data. Cultures were inoculated into 500 ml shake flasks containing 60 ml TB-animal free (TBAF) broth Teknova, cat #T7660) with 50 mM MOPS (Teknova cat #M8405) and 50 μg/ml kanamycin (Teknova, cat #K2125) from colony or glycerol stocks as indicated and incubated at 37° C., 300 rpm. Growth was measured using absorbance at 600 nm and plasmid yields were obtained by alkaline lysis of cell pellets from each culture and UPLC analysis.

Assaying Plasmid Productivity of E. coli Strains in Ambr250 Bioreactors

Seed Fermentation: For the seed fermentation, the media was prepared by adding 1 mL of 50 mg/ml Kanamycin stock and 100 μL of 10% antifoam 204 per liter of TBAF media. To a 125 mL baffled shake flask, 18.75 mL seed media was added aseptically and inoculated with 94 μL of thawed inoculum from glycerol stock. The seed flask was incubated in a shaker incubator for 4-5 hours at 37° C. and 250 RPM (1″ orbital diameter) until the OD600 of 0.6-0.8 was reached (targeting mid-exponential growth). This seed culture was forwarded to inoculate AMBR vessels at 0.1% (v/v) inoculum.

Production Fermentation: The base media for fermentation was TBAF with 1 ml/L of 50 mg/ml Kanamycin. In each AMBR vessel, 160 mL of this media was aseptically added and batched with 16 mL of 50% sterile glycerol (60 g/L glycerol batch) and 1mL of 10% sterile antifoam 204. The pH during fermentation was maintained at 7.3±0.1 by using 15% Ammonium hydroxide and 50% (v/v) glycerol (pH stat carbon source feeding). The temperature was maintained at 37±0.5° C. throughout the fermentation. The dissolved oxygen (DO) was maintained at 30% saturation using agitation ramp from 700 to 3000 RPM followed by oxygen enrichment from 21-40%. The airflow is maintained constant at 1.0 VVM throughout the fermentation. At 12 hours EFT, a TBAF feed was started at 2 ml/h. Samples were taken from each vessel at regular intervals for plasmid DNA measurement (using miniprep followed by Nanodrop), biomass measurement (OD600 and g/l wet-cell weight (WCW)) and residual metabolite analyses (glycerol, acetate, phosphate, and ammonia).

Assaying Plasmid Copy Numbers of E. coli Strains Harboring Manufacturing Plasmid(s)

Plasmid copy number (PCN) was determined using TaqMan-based (Life Technologies) quantitative-PCR (qPCR) method as follows. Briefly, E. coli culture was spun down, resuspended in water and diluted (10⁻¹→10⁻⁷). After dilution, samples are heated to 98° C. for 10 minutes for lysis of cells prior to being transferred to qPCR plate containing enzyme mix, primers and probes. PCN is determined by the ΔΔCt method (difference in Ct value for plasmid and genomic DNA at a given dilution) as well as by using plasmid DNA and E. coli gDNA standard curves to calculate relative ratio of plasmid:genomic DNA.

### Construction of Knockout Cassettes

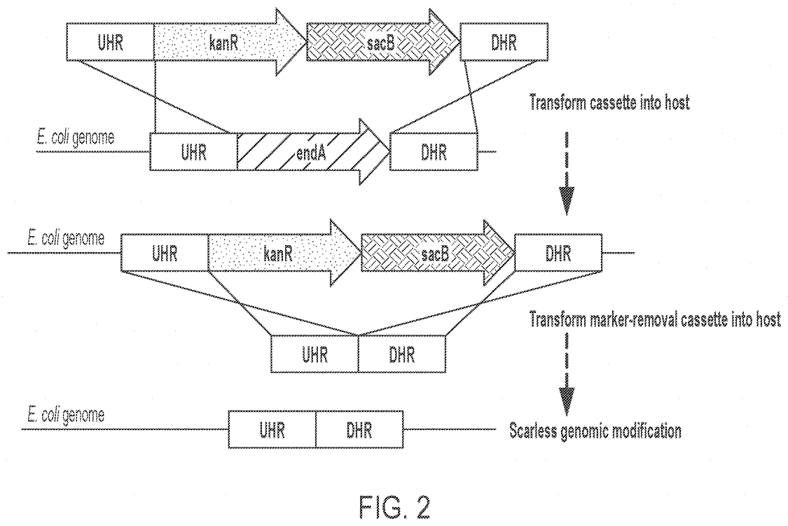

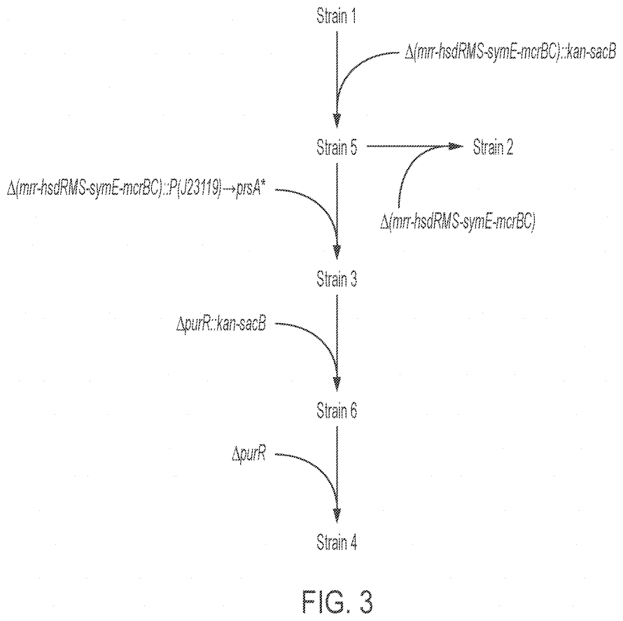

Knockout cassettes for strain engineering work included a DNA cassette that encodes a kanamycin resistance marker (kan) in addition to sacB (encoding the enzyme levansucrase) for negative selection. To allow for the integration of this kan-sacB knockout cassette into the correct location of the genome, small 45-bp upstream and downstream homologous regions were appended onto the knockout cassette using PCR (FIG. 2) and Herculase II DNA polymerase (Agilent, Cat #600697). The knockout cassette was amplified from an internally-produced plasmid containing the kan-sacB cassette, Plasmid 5 (FIG. 3).

Introduction of scar-Less Genomic Deletions in E. coli

The strain that was to be genetically modified was first transformed with Plasmid 6 (FIG. 3) and transformants selected for by plating onto LB-animal free (LBAF) agar containing 100 μg/ml carbenicillin (Teknova, Cat #L1092). A single transformant was then grown up in LBAF broth (Teknova cat #L8900-06) containing 100 μg/ml carbenicillin at 30° C. for 16 hours followed by transferring 30 μl of this overnight culture into a test tube containing 3 ml LBAF broth with 100 μg/mlcarbenicillin (Teknova, Cat #C2135) and incubated for 2 hours at 30° C., 250 rpm. After 2 hours of incubation, expression of the genes encoding a lambda red system and a codon optimized E. coli recA were induced using 100 ng/ml anhydrotetracycline (aTc, Fisher #AC233131000) and 1 mM isopropyl β-D-1-thiogalactopyranoside (IPTG, Millipore cat #70527-3), respectively. After 2-3 additional hours of shaking incubation at 30° C., when OD₆₀₀˜0.6-1.0, 1 ml of culture was harvested to prepare 0.1 ml electrocompetent cells. Fifty ul of electrocompetent cells were mixed with 1 μg of purified knockout cassette and electroporated in 1 mm gapped cuvettes at 1800 volts. Transformations were rescued in 1 ml SOC media (NEB cat #B9020S) at 30° C., 300 rpm for 2 hours then plated onto LBAF agar containing 50 μg/ml kanamycin and 100 μg/ml carbenicillin (Teknova, cat #L3819) and incubated overnight at 30° C. Colony PCR (cPCR) with LongAmp Taq DNA polymerase (NEB, cat #M0287L) was then utilized to screen for primary integrants using a universal primer that binds to the kanamycin resistance gene, kan, and a location-specific primer that binds upstream of the gene targeted for knockout. In parallel with cPCR, the same clones were spotted onto LBAF agar containing 35 μg/ml kanamycin and 100 μg/ml carbenicillin and LB agar containing 60 g/l sucrose (Teknova, cat #L1143). These plates were incubated overnight at 30° C. After confirmation of primary integrants by cPCR, the sucrose sensitivity was confirmed by visually checking for a “no growth” phenotype where the clone were spotted onto LBAF agar containing 60 g/l sucrose. Once a primary integration clone was confirmed by cPCR and was also confirmed to be sucrose-sensitive, the knockout cassette was removed using a similar approach as described below.

To remove a given knockout cassette and obtain a scar-less deletion, a linear dsDNA fragment (“popout cassette”) containing only the UHR and DHR regions was amplified from gBlocks (IDT) and primers. Confirmed primary integrants were grown up in LBAF broth containing 100 μg/ml carbenicillin and 50 μg/ml kanamycin at 30° C. for 16 hours followed by transferring 30 ul of this overnight culture into a test tube containing 3 ml LBAF broth with 100 μg/ml carbenicillin and 50 μg/mlkanamycin and incubated for 2 hours at 30° C., 250 rpm. After 2 hours of incubation, expression of the genes encoding the lambda red system and codon optimized E. coli recA were induced using 100 ng/ml aTc and 1 mM IPTG, respectively. After 2-3 additional hours of shaking incubation at 30° C., when OD₆₀₀˜0.6-1.0, 1 ml of culture was harvested to prepare 0.1 ml electrocompetent cells. Fifty ul of electrocompetent cells were mixed with 1 μg of purified popout cassette, and electroporated in 1 mm gapped cuvettes at 1800 volts. Transformations were rescued in 1 ml SOC media at 30° C., 300 rpm for 2 hours then transferred to a 125 ml shake flask containing 9 ml LBAF broth. This diluted culture was then grown at 30° C., 300 rpm for 5-16 hours followed by transferring 50 ul of culture into a test tube containing 5 ml LBAF-no salt broth containing sucrose (10 g/l soytone (BD Biosciences, cat #243620), 5 g/l yeast extract (Fisher Scientific, cat #DF210929), 60 g/l sucrose (Fisher Scientific, cat #S5-500). The diluted culture was then filter sterilized with 0.2 uM filter Corning #430769). This sucrose-containing culture was then incubated at 30° C., 250 rpm overnight (-16 hours), diluted by 10⁻⁶ in sterile LBAF broth, plated onto LBAF agar (200 μl plated), and incubated overnight at 37° C.

Once isolated colonies were obtained on LBAF agar plate, clones were screened for successful removal of the knockout cassette (kan-sacB) using cPCR and primers that bind upstream and downstream of the gene(s) to be knocked out. In parallel, the clones were replica-spotted onto LBAF agar and LBAF agar containing 100 μg/ml carbenicillin. These plates were incubated overnight (16 hours) at 30° C. to confirm loss of the temperature-sensitive plasmid needed for genome editing, Plasmid 6. For construction of Strain 3, a linear ‘popout cassette’ containing UHR_P(J23119)→prsA_D128A_DHR (UHR and DHR are specific to regions flanking mrr-hsdRMS-symE-mcrBC locus) was used to simultaneously remove the kan-sacB knockout cassette in Strain 5 and allow for constitutive expression of prsA_D128A (prsA*).

Determining poly-A Tail Stability in Strain 4

To determine the post-transformation poly-A tail stability, 50 μl of Strain 4 or control strain chemically-competent cells were transformed with circular plasmids Plasmids 1 and 2. Transformations were rescued in 1 ml SOC for 1 hour at 30° C., 300 rpm and plated on LBAF agar with 50 μg/ml kanamycin. 96 colonies for each transformation were picked into 500 ul TBAF+50 μg/ml kanamycin and grown up for 16 hours at 37° C., 300 rpm. Plasmid DNA was isolated and sent out for sanger sequencing of the poly-A tail. Sequencing data was then analyzed using CNN analysis (developed internally) to quantify % of clones with high-probability of possessing poly-A tails.

To determine poly-A tail stability over many generations of growth, strains Strain 4, Strain 1 and control strain harboring Plasmid 2 (SEQ ID NO: 20) were picked from colonies into test tubes containing 5 ml TBAF with 50 μg/ml kanamycin and incubated at 37° C., 300 rpm for 16-24 hours. The following day, cultures were sampled for OD₆₀₀ and to isolate plasmid DNA. Then, 1 μl of each culture was used to inoculate another set of 5 ml LBAF with 50 μg/ml kanamycin test tubes. This process was repeated for 6 days. Plasmid DNA from each strain was isolated by mini-prep (Qiagen) and samples from each time-point were sent out for sequencing of the poly-A tails. Poly-A tail lengths were determined using Sanger Sequencing and have no more than 5 bases with CV scores <30.

### Generation of Glycerol Stocks and Competent Cell Banks

For creation of glycerol stocks for long-term storage, Strain 3 and Strain 4 were struck out from glycerol stock onto LBAF agar and incubated overnight at 30° C. Single colonies for each strain were inoculated into 3 ml TBAF broth in a 1-liter baffled, shake flask and incubated 16 hours at 30° C., 250 rpm. The following day, 100 ml TBAF broth in 1-liter baffled, shake flask were inoculated to OD₆₀₀=0.05 and incubated at 30° C., 250 rpm for 4-6 hours until OD₆₀₀˜0.6. At this target OD₆₀₀, 50 ml sterile 50% glycerol was added to each culture, mixed and 700 μl aliquoted into 1 ml FluidX tubes and stored at −80° C. A single tube of each lot was thawed to determine viability by plating dilutions onto LBAF agar and incubated overnight at 30° C.

For creation of competent cell banks, Strain 3 and Strain 4 were struck out from glycerol stock onto LBAF agar and incubated overnight at 30° C. Single colonies for each strain were inoculated into 100 ml animal-free SOB broth (Teknova, cat #S2615) in a 1-liter baffled, shake flask and incubated for 30 hours at 18° C., 250 rpm. When a target OD₆₀₀˜0.2 was achieved, cells were harvested, washed, and aliquoted into sterile, FluidX tubes (50 μl per tube).

Transformation efficiency was determined by average transformation efficiency obtained when 10 ng of Plasmid 1 (SEQ ID NO: 19) is transformed into 50 μl competent cells (n=2) for 30 seconds at 42° C., followed by 2-minute hold at 4° C. 0.95 ml SOC was added to cells then vials were incubated at 30 C, 250 rpm for 1 hour prior to plating on LBAF agar containing 50 μg/ml kanamycin.

Culture purity was determined by spreading 75 μL of each competent cell strain, Strain 3 and Strain 4, onto both lx tryptic soy agar (TSA) and lx Sabouraud dextrose agar (SDA) plates, incubating TSA at 30° C. and SDA at 22° C. for 3-5 days, and, after, visually inspecting plates for any adventitious growth of microorganisms. There was no visible contaminant growth on all plates after 76 hours of incubation.

### Results

Construction of strains Strain 2, Strain 3 and Strain 4

Strain 1 (Escherichia coli MG1655 ΔendA ΔrecA) was used as the parental strain to create Strain 2, Strain 3, and Strain 4 as shown in FIG. 3. All desired genetic alterations to the genome were performed as described in methods section and confirmed by PCR. All final strains were confirmed to be kanamycin-sensitive, carbenicillin-sensitive and sucrose-insensitive. In addition, PCR products generated to confirm the new genotypes were sanger sequenced. All strains were confirmed to have the correct, intended DNA sequences at the genomic loci that have been altered.

### Removing EcoKI Restriction System Improves Transformation Efficiency—Strain 2

Wild-type E. coli K12 strains (such as the parent of Strain 1) possess a native restriction endonuclease system (EcoKI) that degrades non-methylated DNA with unique EcoKI restriction site(s). The EcoKI restriction system in Strain 1 was successfully removed, yielding strain Strain 2. Once completed, it was confirmed that the desired phenotype was obtained by attempting transformation of Strain 1 and Strain 2 with a methylated and non-methylated plasmid that contains three EcoKI sites. Transformation of the methylated plasmid into Strain 1 yields a lawn of bacteria whereas, when the same non-methylated plasmid is transformed into Strain 1, no colonies were obtained demonstrating the potentially severe negative impact of the EcoKI system on transformation efficiency. Contrary to Strain 1, Strain 2 demonstrates similar transformation efficiencies with either methylated or non-methylated plasmid as EcoKI has been removed. This allows the use of Strain 2 and its descendants in cell banking workflows as well as higher-throughput cloning platforms such as pre-clinical DNA and PVU as Strain 2 will accept plasmid DNA from methylation-deficient hosts (such as control strain) or DNA that is cloned using gBlocks or PCR products (non-methylated DNA fragments).

### Overexpression of PrsA* in Strain 1 Increases Plasmid Yield in Shake Flasks

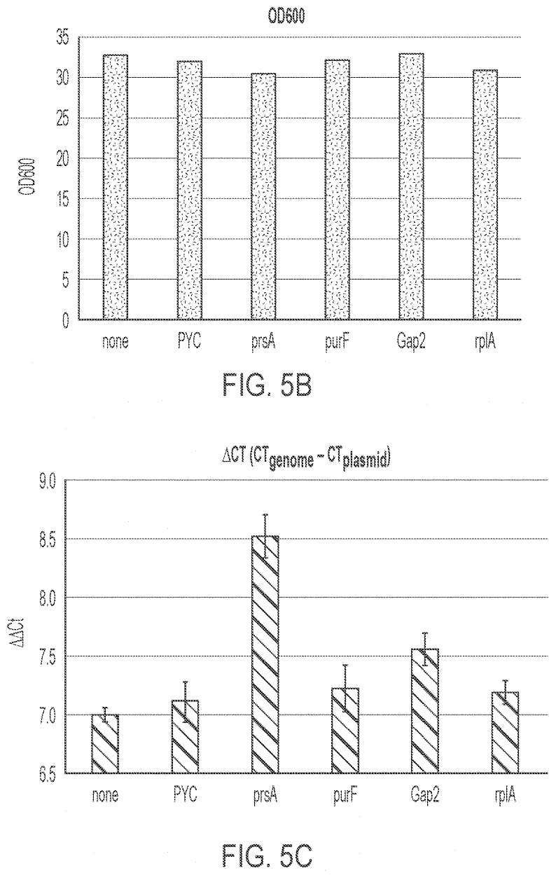

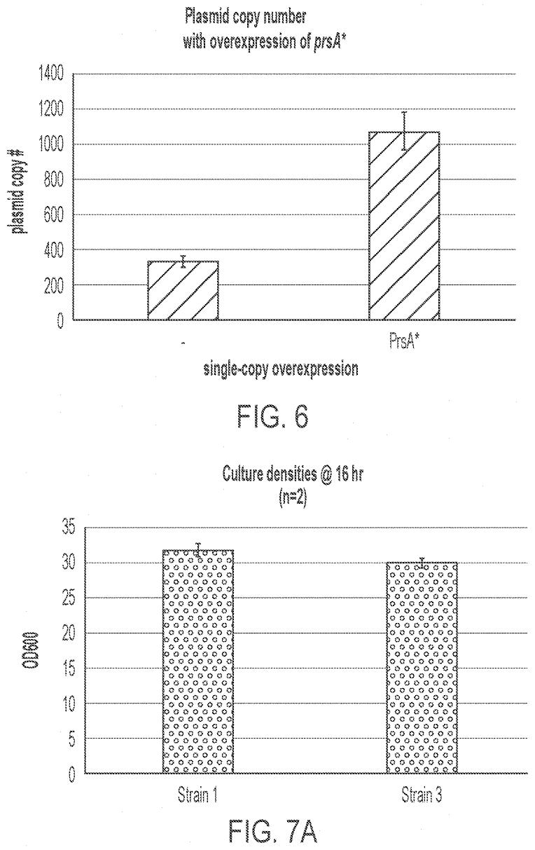

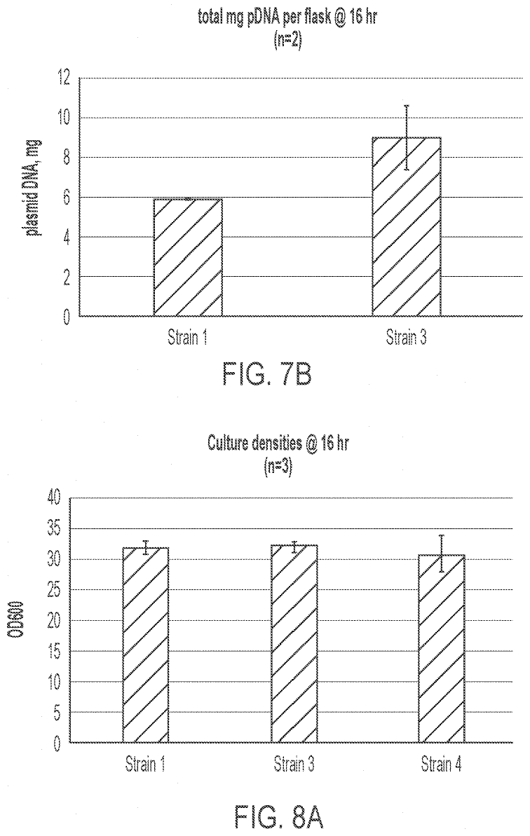

Gene targets were identified for overexpression that result in increased plasmid DNA yield. A panel of single-copy overexpression plasmids, each carrying a unique codon-optimized gene as shown in FIGS. 5A-5C, was tested using Strain 1 harboring Plasmid 1 (SEQ ID NO: 19) as a host to determine if any of the tested genes may be synthetically overexpressed to increase copy number of a representative manufacturing plasmid. Growth and plasmid DNA yields were tested and, as shown in FIGS. 5A-5C, overexpression of prsA* significantly increased plasmid DNA yield (FIGS. 5A-5C) and copy number (FIG. 6). This variant enzyme possesses a mutation that removes feedback-inhibition by ADP, thereby de-regulating a key step that provides a metabolite for purine and pyrimidine synthesis, PRPP. To create a plasmid-free strain that possesses stable expression of this prsA variant, a constitutive expression cassette was integrated in place of the EcoKI system using the Strain 14(mrr-hsdRMS-symE-mcrBC)::kan-sacB intermediate strain that was created when producing Strain 2. The resulting strain, Strain 3, is a descendant of Strain 1 that has had its EcoKI restriction-encoding locus replaced with constitutive expression of prsA* from the genome. Growth and plasmid productivity of Strain 3 relative to Strain 1 and NEB stable were assayed in shake flasks. Similar to what was observed when prsA* is expressed from single-copy plasmid, plasmid DNA yield increased significantly in Strain 3 relative to its parent, Strain 1 (FIGS. 7A-7B).

Inactivation of purR and Overexpression of prsA* Further Improves Plasmid Yield in Shake Flasks

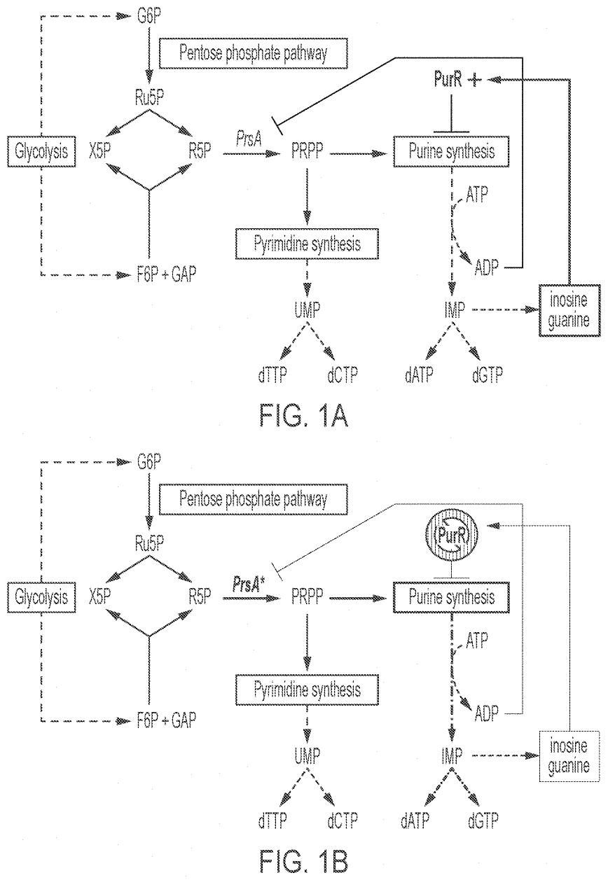

It was an aim to de-repress the 32 genes that encode the enzymes for nucleotide biosynthesis by removing the transcriptional repressor, PurR (FIGS. 1A-1B), from Strain 3. The conformational change that occurs when PurR associates with guanine and hypoxanthine (products of purine synthesis pathway) allows the enzyme to bind to promoters of its regulon, resulting in transcriptional repression. The resulting strain lacking purR, Strain 4, thereby possesses a higher carbon flux capacity over its parent, Strain 3, for nucleotide synthesis. Flask studies performed with Strain 1, Strain 3 and Strain 4 have indeed shown higher plasmid DNA yields for the latest strains (FIGS. 8A-8B) (Strain 1<Strain 3<Strain 4). All strains tested grew well and produced similar final culture densities.

Strain 3 and Strain 4 Display Higher Plasmid DNA Yields in Comparison with Strain 1 in Ambr250 Bioreactors

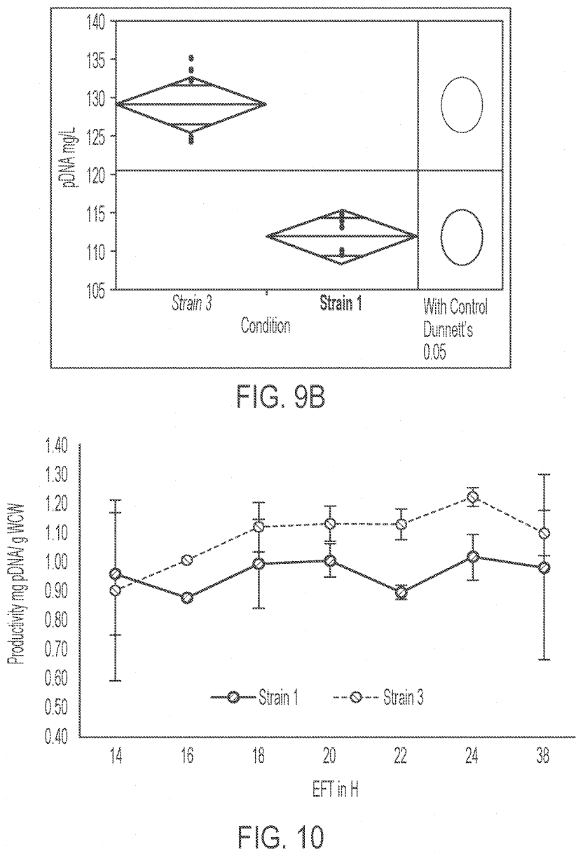

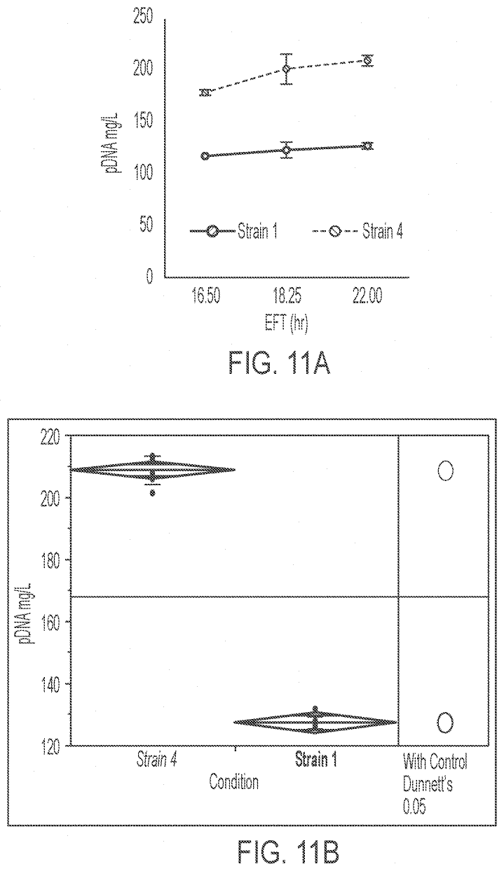

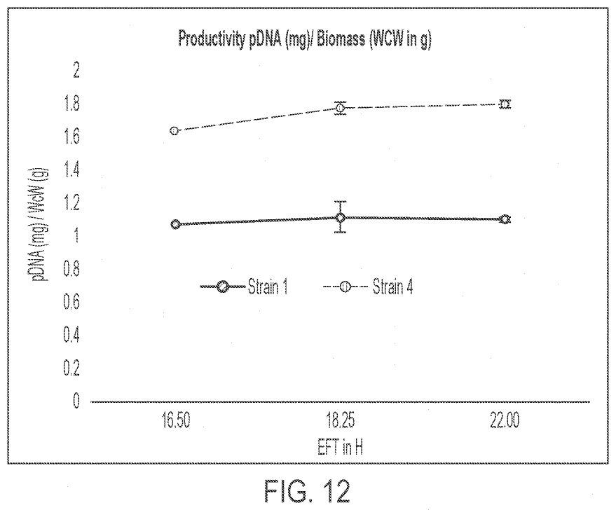

FIG. 9A shows a kinetic profile for pDNA production. A statistical analysis of the pDNA produced at 22-hour EFT is shown in FIG. 9B, which shows Strain 3 is statistically higher than Strain 1 at 95% confidence interval (the two strains were compared using Control Dunnett's test for comparing means). Both strains produced comparable biomass. Hence the specific productivity, calculated as pDNA produced (mg/L) per gram biomass (measured as WCW g/L), for Strain 3 was higher than Strain 1 (up to ˜1.2× higher) (FIG. 10). Fermentation results comparing pDNA productivities of Strain 4 and Strain 1 (FIGS. 11A-1 B) showed that Strain 4 produces more pDNA than Strain 1 at all timepoints after 16 hours EFT. Both strains Strain 4 and Strain 1 produced comparable biomass. Hence, the specific productivity of Strain 4, calculated as pDNA produced (mg/L) per gram biomass (measured as WCW g/L), was significantly higher than Strain 1 (up to 1.8× higher) (FIG. 12). In summary, both Strain 3 and Strain 4 demonstrated significantly higher specific plasmid DNA productivities over the parent, Strain 1, in Ambr250 bioreactors with Strain 4 as the most productive strain (Strain 1<Strain 3<Strain 4).

### Strain 4 Displays Improved Poly-A Tail Stability Compared to NEBSstable When Transformed With Circular Plasmid

To confirm that Strain 4 maintains the poly-A tail within desired length specifications (95-105 bp), 2 different plasmids containing high-quality poly-A tails were transformed into Strain 4 and NEB stable as comparison. After growing up 96 colonies from each transformation, plasmid DNAs were isolated and the poly-A tail was sequenced. An algorithm for determining clones with high probability of passing tails (tails that are within length and purity specs) was used to analyze the sanger sequencing data generated in this experiment. As shown below, clones picked from Strain 4 were significantly more-probable to have passing poly-A tails as compared to NEB stable.

TABLE 4
Percent (%) of transformants having high-probability of passing poly-A tails

| | | % of population with high- |
|---|---|---|
| Strain | Plasmid | probabililty of passing poly-A tail |
| Control Strain | Plasmid 2 | 11 |
| | Plasmid 1 | 60 |
| Strain 4 | Plasmid 2 | 72 |
| | Plasmid 1 | 86 |

### Strain 4 Maintains Poly-A Tail Stability Over Many Generations of Growth

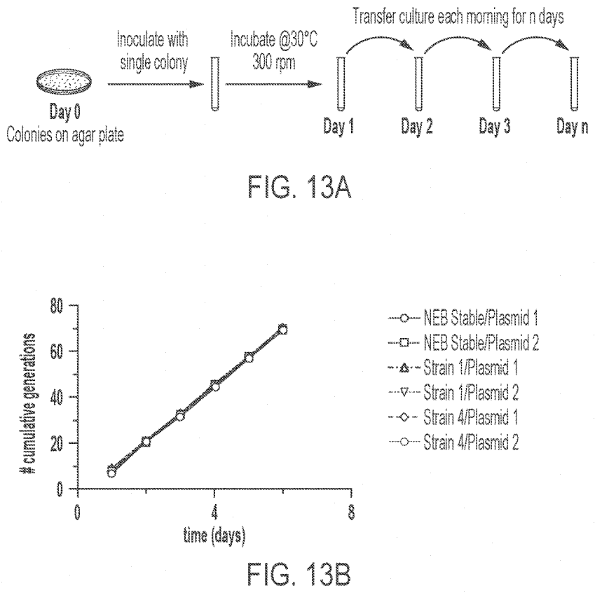

In addition to maintaining the poly-A tail after transformation, the long-term poly-A stability in Strain 4 was characterized in comparison with NEB stable. For Strain 4, a loss of 3-5 base pairs of the poly-A tail was observed after approximately 69 generations (Table 5, FIGS. 13A-13C). These results are comparable to those historically obtained with NEB stable and Strain 1 indicating that there is comparable long-term tail stability in Strain 4, Strain 1 and NEB stable. Importantly, no tail heterogeneity was observed after 69 generations of growth. About 50 generations of growth at large-scale pDNA production process at 30-liter or 300L fermentation scales (FIG. 13C), these data support the use of Strain 4 as a host for pDNA production.

TABLE 5
Tail lengths of various plasmids in NEB stable, Strain 1 and Strain 4 after 69
generations of population with high-probabililty of passing poly-A tail:

| Strain | Plasmid | Day 0 | Day 6 |
|---|---|---|---|
| NEB Stable | Plasmid 2 | 94 | 92 |
| | Plasmid 2 | 94 | 92 |
| | Plasmid 1 | 85 | 84 |
| | Plasmid 1 | 85 | 71 |
| Strain 1 | Plasmid 2 | 91 | 92 |
| | Plasmid 2 | 91 | 93 |
| | Plasmid 1 | 93 | 91 |
| | Plasmid 1 | 93 | 90 |
| Strain 4 | Plasmid 2 | 97 | 92 |
| | Plasmid 2 | 97 | 93 |
| | Plasmid 1 | 95 | 92 |
| | Plasmid 1 | 95 | 93 |

### Growth Profiles of Strain 4 for Large-Scale Plasmid DNA

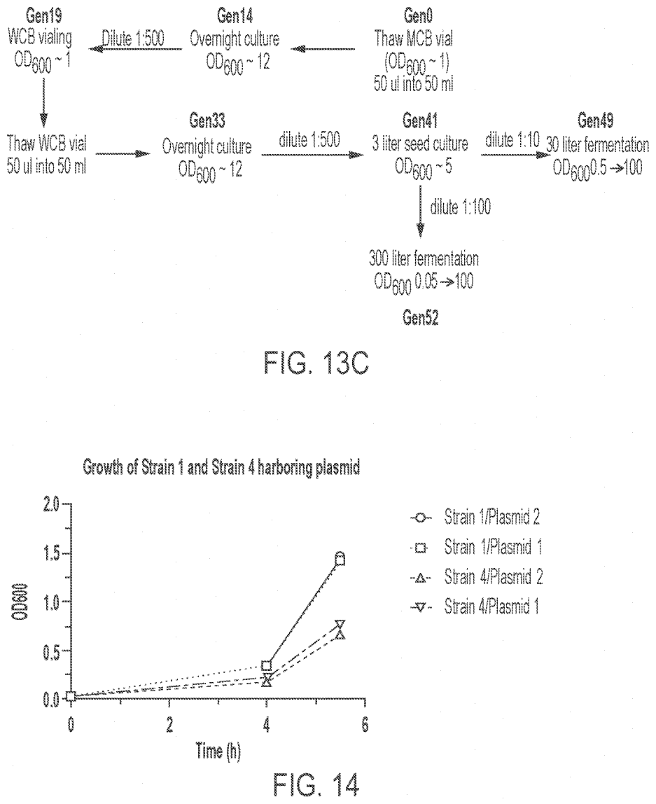

Strain 4 and Strain 1 harboring the indicated plasmids were inoculated into shake flasks to evaluate its growth profile in comparison with Strain 1. As shown in FIG. 14, Strain 4 displays a longer growth-lag; however, the difference is small.

### Generation of Competent Cells Banks of Strain 3 and Strain 4

Strain 3 and Strain 4 were grown up from colony in aseptic conditions in LBAF broth and ninety-six, 1 ml FluidX tubes (1 lot) for each strain were filled and stored @ −80° C. In addition, a lot of chemically competent cells was created for each strain as described in methods section. These lots were QC tested for the presence of phage using Mitomycin C induction assay and tested to confirm strain purity. No phage was detected and purity of each tested lot was confirmed (Table 6). Transformation efficiencies obtained were sufficient for use and were comparable to those obtained using Strain 1.

TABLE 6
Glycerol stock and competent cell lots of Strain 3 and Strain 4

| | | Viability | Transformation efficiency |
|---|---|---|---|
| Strain | Glycerol stock/comp cells | CFU/ml | CFU/ml |
| 3 | Glycerol stock | 7.6 × 10⁷ | NA |
| 3 | Competent Cells | ND | 2.3 × 10⁵ |
| 4 | Glycerol stock | 1.4 × 10⁸ | NA |
| 4 | Competent Cells | ND | 3.2 × 10⁵ |

‘ND’ denotes ‘no data’, ‘NA’ denotes ‘not applicable’

### CONCLUSION

New strains of E. coli were created that demonstrate improved cloning efficiency for use in high-throughput cloning processes and higher plasmid DNA yield. The EcoKI restriction system was removed from Strain 1, which allows for efficient transformation efficiencies with non-methylated DNA (e.g., gBlocks, PCR products and circular plasmid isolated from NEB stable). Next, some additional genomic modifications were introduced that resulted in the upregulation of the nucleotide biosynthesis pathways. The final strain, Strain 4, will readily accept non-methylated plasmid DNA isolated from NEB stable or DNA from a Gibson assembly reaction using synthesized or PCR gene fragments. As shown herein, Strain 4 also demonstrates significantly higher plasmid DNA productivity (1.8×-2×) as compared to the parental strain, Strain 1, in shake flasks and in Ambr250 bioreactors that mimic large-scale GMP fermentation process. Further characterization of Strain 4 has also shown that the strain demonstrates improved poly-A tail-stability compared to NEB stable at the transformation event and maintains purity of the poly-A tail over many generations of growth.

### Example 2: Mutations to Plasmid Replication Machinery Impacts Plasmid Production and Replication Efficiency

Mutations were made to the pUC origin of replication (SEQ ID NO: 16) in the control plasmid PL_007984 (SEQ ID NO: 19) with the goal of increasing plasmid titers. Engineered segments of the pUC type origin of replication were synthesized as double-stranded DNA fragments (IDT gBlocks). Parent plasmid was digested with BssHII and ApaLI and the gBlocks were cloned into the plasmid by Gibson assembly. The new variant plasmids were sequence-confirmed. The mutations made and tested were created by introduction of specific sequences of partial RNAII/I and are shown as SEQ ID NO: 1-15 herein.

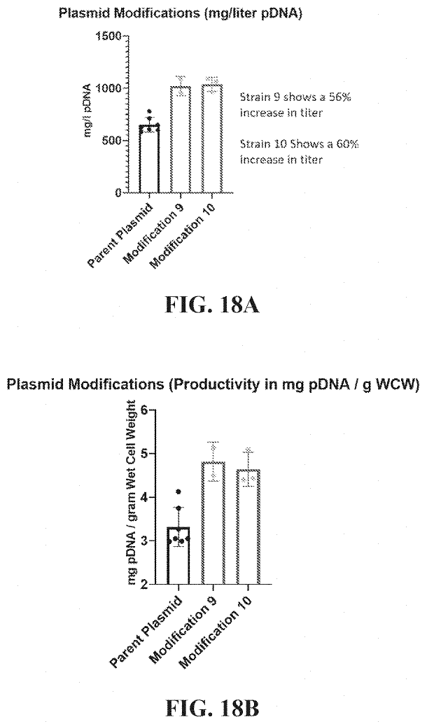

Replication was assessed by measurement of plasmid production as mg/liter of pDNA. Modification 9 (Ori10; SEQ ID NO: 10) includes a deletion early in the RNAII transcribed region. Modifications 9 and 10 (SEQ ID NO: 10-11, respectively) showed significantly increased titers (56%/60% increases respectively; FIG. 18A). Strains containing Modifications 9 and 10 (SEQ ID NO: 10-11, respectively) also showed productivity improvements. As shown in FIG. 18B, Modifications 9 and 10 (SEQ ID NO: 10-11, respectively) had greater weight of pDNA per gram of wet cell weight than the parent plasmid (Plasmid 1; SEQ ID NO: 19)

TABLE 7
Exemplary Sequences

| SEQ | | |
|---|---|---|
| ID | | |
| NO | SEQUENCE | DESCRIPTION |
| 1 | aaatcccttaacgtgagttacgcgcgcgtcgttccactgagcgtc | Ori1 |
| | agaccccgtagaaaagatcaaaggatcttcttgaAatcctttttt | |
| | tctgcgcgtaatctgctgcttgcaaaaaaaaaaccaccgctacca | |
| | gcggtggtttgtttgccggatcaagagctaccaactctttttccg | |
| | aaggtaactggcttcagcagagcgcagataccaaatactgttctt | |
| | ctagtgtagccgtagttagcccaccacttcaagaactctgtagca | |
| | ccgcctacatacctcgctctgctaatcctgttaccagtggctgct | |
| | gccagtggcgataagtcgtgtcttaccgggttggactcaagacga | |
| | tagttaccggataaggcgcagcggtcgg | |
| 2 | aaatcccttaacgtgagttacgcgcgcgtcgttccactgagcgtc | Ori2 |
| | agaccccgtagaaaagatcaaaggatcttcttgagatcctttttt | |
| | tctgcgcgtaatAtgctgcttgcaaacaaaaaaaccaccgctacc | |
| | agcggtggtttgtttgccggatcaagagctaccaactctttttcc | |
| | gaaggtaactggcttcagcagagcgcagataccaaatactgttct | |
| | tctagtgtagccgtagttagcccaccacttcaagaaataagtcgt | |
| | gtcttaccgggttggactcaagacgatagttaccggataaggcgc | |
| | agcggtcgggctgaacggggggttcgtgcacacagcccagcttgg | |
| | agcgaacgac | |
| 3 | aaatcccttaacgtgagttacgcgcgcgtcgttccactgagcgtc | Ori3 |
| | agaccccgtagaaaagatcaaaggatcttcttgagatcctttttt | |
| | tctgcgcgtaatctgctgcttgcaaacaaaaaaaAcaccgctacc | |
| | agcggtggtttgtttgccggatcaagagctaccaactctttttcc | |
| | gaaggtaactggcttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgac | |
| 4 | aaatcccttaacgtgagttacgcgcgcgtcgttccactgagcgtc | Ori4 |
| | agaccccgtagaaaagatcaaaggatcttcttgagatcctttttt | |
| | tctgcgcgtaatctgctgcttgcaaacaaaaaaaccaccgTtacc | |
| | agcggtggtttgtttgccggatcaagagctaccaactctttttcc | |
| | gaaggtaactggcttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgac | |
| 5 | aaaggatcttcttgagatcctttttttctgcgcgtaatctgctgc | Ori5 |
| | ttgcaaacaaaaaaaccaccgaaatcccttaacgtgagttacgcg | |
| | cgcgtcgttccactgagcgtcagaccccgtagaaaagatcaaagg | |
| | atcttcttgagatcctttttttctgcgcgtaatctgctgcttgca | |
| | aacaaaaaaaccaccgctaTcagcggtggtttgtttgccggatca | |
| | agagctaccaactctttttccgaaggtaactggcttcagcagagc | |
| | gcagataccaaatactgttcttctagtgtagccgtagttagccca | |
| | ccacttcaagaaataagtcgtgtcttaccgggttggactcaagac | |
| | gatagttaccggataaggcgcagcggtcgggctgaacggggggtt | |
| | cgtgcacacagcccagcttggagcgaacgac | |
| 6 | aaatcccttaacgtgagttacgcgcgcgtcgttccactgagcgtc | Ori6 |
| | agaccccgtagaaaagatcaaaggatcttcttgagatcctttttt | |
| | tctgcgcgtaatctgctgcttgcaaacaaaaaaaccaccgctacc | |
| | Ggcggtggtttgtttgccggatcaagagctaccaactctttttcc | |
| | gaaggtaactggcttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgacaaatcccttaacgtg | |
| | agttacgcgcgcgtcgttccactgagcgtcagaccccgtagaaaa | |
| | gatcaaaggatcttcttgagatcctttttttctgcgcgtaatctg | |
| | ctgcttgcaaacaaaaaaaccaccgctaccagcggtgtttgtttg | |
| 7 | ccggatcaagagctaccaactctttttccgaaggtaactggcttc | Ori7 |
| | agcagagcgcagataccaaatactgttcttctagtgtagccgtag | |
| | ttagcccaccacttcaagaactctgtagcaccgcctacatacctc | |
| | gctctgctaatcctgttaccagtggctgctgccagtggcgataag | |
| | tcgtgtcttaccgggttggactcaagacgatagttaccggataag | |
| | gcgcagcggtcgggctgaacggggggttcgtgcacacagcccagc | |
| | ttggagcgaacgac | |
| 8 | aaatcccttaacgtgagttacgcgcgcgtcgttccactgagcgtc | Ori8 |
| | agaccccgtagaaaagatcaaaggatcttcttgagatcctttttt | |
| | tctgcgcgtaatctgctgcttgcaaacaaaaaaaccaccgctacc | |
| | agcggtggtttgtttgccggatcaagagTtaccaactctttttcc | |
| | gaaggtaactggcttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgac | |
| 9 | aaatcccttaacgtgagttacgcgcgcgtcgttccactgagcgtc | Ori9 |
| | agaccccgtagaaaagatcaaaggatcttcttgagatcctttttt | |
| | tctgcgcgtaatctgctgcttgcaaacaaaaaaaccaccgctacc | |
| | agcggtggtttgtttgccggatcaagagctaTcaactctttttcc | |
| | gaaggtaactggcttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgac | |
| 10 | aaatcccttaacgtgagttacgcgcgcgtcgttccactgagcgtc | Ori10 |
| | agaccccgtagaaaagatcaaaggatcttcttgagatcctttttt | |
| | tctgcgcgtaatctgctgcttgcaaacaaaaaaaccaccg ctac | |
| | cagcggtggtttgtttgccggatcaagagctacTaactctttttc | |
| | cgaaggtaactggcttcagcagagcgcagataccaaatactgttc | |
| | ttctagtgtagccgtagttagcccaccacttcaagaactctgtag | |
| | caccgcctacatacctcgctctgctaatcctgttaccagtggctg | |
| | ctgccagtggcgataagtcgtgtcttaccgggttggactcaagac | |
| | gatagttaccggataaggcgcagcggtcgggctgaacggggggtt | |
| | cgtgcacacagcccagcttggagcgaacgac | |
| 11 | aaaggatcttcttgagatcctttttttctgcgcgtaatctgctgc | Ori11 |
| | ttgcaaacaaaaaaaccaccgaaaggatcttcttgagactccttt | |
| | ttttctgcgcgttatctgctgcttgcaaacaaaaaaaccaccgct | |
| | accagcggtggtttgtttgccggatcaagagctacTaactctttt | |
| | tccgaaggtaactggcttcagcagagcgcagataccaaatactgt | |
| | tcttctagtgtagccgtagttagcccaccacttcaagaactctgt | |
| | agcaccgcctacatacctcgctctgctaatcctgttaccagtggc | |
| | tgctgccagtggcgataagtcgtgtcttaccgggttggactcaag | |
| | acgatagttaccggataaggcgcagcggtcgggctgaacgggggg | |
| | ttcgtgcacacagcccagcttggagcgaacgac | |
| 12 | aaatcccttaacgtgagttacgcgcgcgtcgttccactgagcgtc | Ori12 |
| | agaccccgtagaaaagatcaaaggatcttcttgagatcctttttt | |
| | tctgcgcgtaatctgctgcttgcaaacaaaaaaaccaccgctacc | |
| | agcggtggtttgtttgccggatcaagagctaccaGctctttttcc | |
| | gaaggtaactggcttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgac | |
| 13 | aaatcccttaacgtgagttacgcgcgcgtcgttccactgagcgtc | Ori13 |
| | agaccccgtagaaaagatcaaaggatcttcttgagatcctttttt | |
| | tctgcgcgtaatctgctgcttgcaaacaaaaaaaccaccgctacc | |
| | agcggtggtttgtttgccggatcaagagctaccaactctttttcc | |
| | gaaggtaactggTttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgac | |
| 14 | aaatcccttaacgtgagttacgcgcgcgtcgttccactgagcgtc | Ori14 |
| | agaccccgtagaaaagatcaaatcccttaacgtgagttacgcgcg | |
| | cgtcgttccactgagcgtcagaccccgtagaaaagatcctaccag | |
| | cggtggtttgtttgccggatcaagagctaccaactctttttccga | |
| | aggtaactggcttcagTagagcgcagataccaaatactgttcttc | |
| | tagtgtagccgtagttagcccaccacttcaagaactctgtagcac | |
| | cgcctacatacctcgctctgctaatcctgttaccagtggctgctg | |
| | ccagtggcgataagtcgtgtcttaccgggttggactcaagacgat | |
| | agttaccggataaggcgcagcggtcgggctgaacggggggttcgt | |
| | gcacacagcccagcttggagcgaacgac | |
| 15 | aaatcccttaacgtgagttacgcgcgcgtcgttccactgagcgtc | Ori15 |
| | agaccccgtagaaaagatcaaatcccttaacgtgagttacgcgcg | |
| | cgtcgttccactgagcgtcagaccccgtagaaaagatcaaaggat | |
| | cttcttgagatcctttttttctgcgcgtaatctgctgcttgcaaa | |
| | caaaaaaaccaccgctaccagcggtggtttgtttgccggatcaag | |
| | agctaccaactctttttccgaaggtaactggcttcagcagagcgc | |
| | agataccaaatactgttcttctagtgtaACCGTAGTCGAGCCACT | |
| | Acagtggcgataagtcgtgtcttaccgggttggactcaagacgat | |
| | agttaccggataaggcgcagcggtcgggctgaacggggggttcgt | |
| | gcacacagcccagcttggagcgaacgac | |
| 16 | aaatcccttaacgtgagttacgcgcgcgtcgttccactgagcgtc | Ori16 |
| | agaccccgtagaaaagatcaaaggatcttcttgagatcctttttt | Parent_Seq_p |
| | tctgcgcgtaatctgctgcttgcaaacaaaaaaaccaccgctacc | UC_ori |
| | agcggtggtttgtttgccggatcaagagctaTcaactctttttcc | |
| | gaaggtaactggcttcagcagagcgcagataccaaatactgttct | |
| | tctagtgtagccgtagttagcccaccacttcaagaactctgtagc | |
| | accgcctacatacctcgctctgctaatcctgttaccagtggctgc | |
| | tgccagtggcgataagtcgtgtcttaccgggttggactcaagacg | |
| | atagttaccggataaggcgcagcggtcgggctgaacggggggttc | |
| | gtgcacacagcccagcttggagcgaacgac | |
| 17 | TATTGAAGATCCGCTTCGATCAGGGGATGGGCTACTGGCGCATCA | KSgBlock94 |
| | ACTTTTCATCGCAATGGCATTACTTTGATTTCCGCGATGACGTTT | DNA |
| | CTTTCCAGTTAGTCAAAATGGCTCAGGCCTGCAAGGAAGGGAATG | sequence |
| | TCGCCAACAGCGAAGAGAGTTGGGCAACGGATGTGCTGGTGGAGG | (5′->3′) |
| | TGATCGCCTCCTGATGATGAGCCGCTCCCGATGTGGTGTCGGGAG | |
| | CGGTATTTTCTATAAAACTTACCGCAATATCAGGCCGGATGCGGC | |
| | TGCGCCTTATCCGGCCCATAACCCCTTACTTCCTCAACCCCGCAA | |
| | ACGCAGCCCGAATCTCTTCCTCCGGCAGCTGGATCCCGATAAACA | |
| | CCATCGTGCTATGCGGTTTTTCATCGCCCCACGGCCTGTCCCAGT | |
| | CGGCGCTGTAGAGGCGCTGGACGCCCTGGAACAGCAGGCGGTTAG | |
| | GTTCGCCGTCAATCCACAGCATCCCTTTGTAACGTAGCAGTTTAT | |
| | CCGCC | |
| 18 | cggcgtaccgcaacacttttgttg | KSgBlock104 |
| | tgcgtaaggtgtgtaaaggcaaa | PurR |
| | cgtttaccttgcgattttgcaggagc | knockout |
| | tgaagttagggtctggagtgaaatgga | in Strain 4 |
| | tcacccgttgcgggagtctcttccggctcccgc | (Entire ORF |
| | agccactccttattcagcgtctcactatc | deleted. |
| | gccgagatactcaagcaaccaggttaacgcaggcgaca | sequence |
| | | upstream: |
| | | single |
| | | underline; |
| | | downstream |
| | | of |
| | | ORF: |
| | | italics) |
| 19 | GGGAAATAAGAGAGAAAAGAAGAGTAAGAAGAAATATAAGAGCCA | Plasmid |
| | CCATGGAAGATGCGAAGAACATCAAGAAGGGACCTGCCCCGTTTT | 1 |
| | ACCCTTTGGAGGACGGTACAGCAGGAGAACAGCTCCACAAGGCGA | (including |
| | TGAAACGCTACGCCCTGGTCCCCGGAACGATTGCGTTTACCGATG | Luciferase |
| | CACATATTGAGGTAGACATCACATACGCAGAATACTTCGAAATGT | as |
| | CGGTGAGGCTGGCGGAAGCGATGAAGAGATATGGTCTTAACACTA | ORF, |
| | ATCACCGCATCGTGGTGTGTTCGGAGAACTCATTGCAGTTTTTCA | which |
| | TGCCGGTCCTTGGAGCACTTTTCATCGGGGTCGCAGTCGCGCCAG | can |
| | CGAACGACATCTACAATGAGCGGGAACTCTTGAATAGCATGGGAA | be |
| | TCTCCCAGCCGACGGTCGTGTTTGTCTCCAAAAAGGGGCTGCAGA | removed) |
| | AAATCCTCAACGTGCAGAAGAAGCTCCCCATTATTCAAAAGATCA | |
| | TCATTATGGATAGCAAGACAGATTACCAAGGGTTCCAGTCGATGT | |
| | ATACCTTTGTGACATCGCATTTGCCGCCAGGGTTTAACGAGTATG | |
| | ACTTCGTCCCCGAGTCATTTGACAGAGATAAAACCATCGCGCTGA | |
| | TTATGAATTCCTCGGGTAGCACCGGTTTGCCAAAGGGGGTGGCGT | |
| | TGCCCCACCGCACTGCTTGTGTGCGGTTCTCGCACGCTAGGGATC | |
| | CTATCTTTGGTAATCAGATCATTCCCGACACAGCAATCCTGTCCG | |
| | TGGTACCTTTTCATCACGGTTTTGGCATGTTCACGACTCTCGGCT | |
| | ATTTGATTTGCGGTTTCAGGGTCGTACTTATGTATCGGTTCGAGG | |
| | AAGAACTGTTTTTGAGATCCTTGCAAGATTACAAGATCCAGTCGG | |
| | CCCTCCTTGTGCCAACGCTTTTCTCATTCTTTGCGAAATCGACAC | |
| | TTATTGATAAGTATGACCTTTCCAATCTGCATGAGATTGCCTCAG | |
| | GGGGAGCGCCGCTTAGCAAGGAAGTCGGGGAGGCAGTGGCCAAGC | |
| | GCTTCCACCTTCCCGGAATTCGGCAGGGATACGGGCTCACGGAGA | |
| | CAACATCCGCGATCCTTATCACGCCCGAGGGTGACGATAAGCCGG | |
| | GAGCCGTCGGAAAAGTGGTCCCCTTCTTTGAAGCCAAGGTCGTAG | |
| | ACCTCGACACGGGAAAAACCCTCGGAGTGAACCAGAGGGGCGAGC | |
| | TCTGCGTGAGAGGGCCGATGATCATGTCAGGTTACGTGAATAACC | |
| | CAGAAGCGACGAATGCGCTGATCGACAAGGATGGGTGGTTGCATT | |
| | CGGGAGACATTGCCTATTGGGATGAGGATGAGCACTTCTTTATCG | |
| | TAGATCGACTTAAGAGCTTGATCAAATACAAAGGCTATCAGGTAG | |
| | CGCCTGCCGAGCTCGAGTCAATCCTGCTCCAGCACCCCAACATTT | |
| | TCGACGCCGGAGTGGCCGGGTTGCCCGATGACGACGCGGGTGAGC | |
| | TGCCAGCGGCCGTGGTAGTCCTCGAACATGGGAAAACAATGACCG | |
| | AAAAGGAGATCGTGGACTACGTAGCATCACAAGTGACGACTGCGA | |
| | AGAAACTGAGGGGAGGGGTAGTCTTTGTGGACGAGGTCCCGAAAG | |
| | GCTTGACTGGGAAGCTTGACGCTCGCAAAATCCGGGAAATCCTGA | |
| | TTAAGGCAAAGAAAGGCGGGAAAATCGCTGTCTGATAATAGGCTG | |
| | GAGCCTCGGTGGCCATGCTTCTTGCCCCTTGGGCCTCCCCCCAGC | |
| | CCCTCCTCCCCTTCCTGCACCCGTACCCCCGTGGTCTTTGAATAA | |
| | AGTCTGAGTGGGCGGCAAAAAAAAAAAAAAAAAAAAAAAAAAAAA | |
| | AAAAAAAAAAAAAAAAAAAAAAAAAAAAAAAAAAAAAAAAAAAAA | |
| | AAAAAAAAAAAAAAAAAAAAAAAAAATCTAGACATCCCTTCAGAG | |
| | TCCCGGGTAGCATAACCCCTTGGGGCCTCTAAACGGGTCTTGAGG | |
| | GGTTTTTTGCGAGCTCGGTACCCAGCCCCGACGAGCTTCATGCCG | |
| | TTAGTCGCACTGCAAGGGGTGTTATGAGCCATATTCAGGTATAAA | |
| | TGGGCTCGCGATAATGTTCAGAATTGGTTAATTGGTTGTAACACT | |
| | GACCCCTATTTGTTTATTTTTCTAAATACATTCAAATATGTATCC | |
| | GCTCATGAGACAATAACCCTGATAAATGCTTCAATAATATTGAAA | |
| | AAGGAAGAATATGAGCCATATTCAACGGGAAACGTCGAGGCCGCG | |
| | ATTAAATTCCAACATGGACGCTGATTTATATGGGTATAAATGGGC | |
| | TCGCGATAATGTCGGGCAATCAGGTGCGACAATCTATCGCTTGTA | |
| | TGGGAAGCCCGATGCGCCAGAGTTGTTTCTGAAACATGGCAAAGG | |
| | TAGCGTTGCCAATGATGTTACAGATGAGATGGTCAGACTAAACTG | |
| | GCTGACGGAATTTATGCCACTTCCGACCATCAAGCATTTTATCCG | |
| | TACTCCTGATGATGCATGGTTACTCACCACTGCGATCCCCGGAAA | |
| | AACAGCGTTCCAGGTATTAGAAGAATATCCTGATTCAGGTGAAAA | |
| | TATTGTTGATGCGCTGGCAGTGTTCCTGCGCCGGTTGCACTCGAT | |
| | TCCTGTTTGTAATTGTCCTTTTAACAGCGATCGCGTCTTCCGTCT | |
| | TGCACAAGCGCAATCACGAATGAATAACGGTTTGGTTGATGCGAG | |
| | TGATTTTGATGACGAGCGTAATGGCTGGCCTGTTGAACAAGTCTG | |
| | GAAAGAAATGCATAAACTTTTGCCATTCTCACCGGATTCAGTCGT | |
| | CACTCATGGTGATTTCTCACTTGATAACCTTATTTTTGACGAGGG | |
| | GAAATTAATAGGTTGTATTGATGTTGGACGAGTCGGAATCGCAGA | |
| | CCGATACCAGGATCTTGCCATTCTATGGAACTGCCTCGGTGAGTT | |
| | TTCTCCTTCATTACAGAAACGGCTTTTTCAAAAATATGGTATTGA | |
| | TAATCCTGATATGAATAAATTGCAGTTTCATTTGATGCTCGATGA | |
| | GTTTTTCTAAGCAGAGCATTACGCTGACTTGACGGGACGGCGCAA | |
| | GCTCATGACCAAAATCCCTTAACGTGAGTTACGCGCGCGTCGTTC | |
| | CACTGAGCGTCAGACCCCGTAGAAAAGATCAAAGGATCTTCTTGA | |
| | GATCCTTTTTTTCTGCGCGTAATCTGCTGCTTGCAAACAAAAAAA | |
| | CCACCGCTACCAGCGGTGGTTTGTTTGCCGGATCAAGAGCTACCA | |
| | ACTCTTTTTCCGAAGGTAACTGGCTTCAGCAGAGCGCAGATACCA | |
| | AATACTGTTCTTCTAGTGTAGCCGTAGTTAGCCCACCACTTCAAG | |
| | AACTCTGTAGCACCGCCTACATACCTCGCTCTGCTAATCCTGTTA | |
| | CCAGTGGCTGCTGCCAGTGGCGATAAGTCGTGTCTTACCGGGTTG | |
| | GACTCAAGACGATAGTTACCGGATAAGGCGCAGCGGTCGGGCTGA | |
| | ACGGGGGGTTCGTGCACACAGCCCAGCTTGGAGCGAACGACCTAC | |
| | ACCGAACTGAGATACCTACAGCGTGAGCTATGAGAAAGCGCCACG | |
| | CTTCCCGAAGGGAGAAAGGCGGACAGGTATCCGGTAAGCGGCAGG | |
| | GTCGGAACAGGAGAGCGCACGAGGGAGCTTCCAGGGGGAAACGCC | |
| | TGGTATCTTTATAGTCCTGTCGGGTTTCGCCACCTCTGACTTGAG | |
| | CGTCGATTTTTGTGATGCTCGTCAGGGGGGCGGAGCCTATGGAAA | |
| | AACGCCAGCAACGCGGCCTTTTTACGGTTCCTGGCCTTTTGCTGG | |
| | CCTTTTGCTCACATGTTCTTTCCTGCGTTATCCCCTGATTCTGTG | |
| | GATAACCGTATTACCGCCTGCTCGCCGCAGCCGAACGACCGAGCG | |
| | CAGCGAGTCAGTGAGCGAGGAAGCGGAAGGCGAGAGTAGGGAACT | |
| | GCCAGGCATCAAACTAAGCAGAAGGCCCCTGACGCATGGCCTTTT | |
| | TGCGTTTCTACAAACTCTTTCTGTGTTGTAAAACGACGGCCAGTC | |
| | TTAAGCTCGGGCCCCTTTTCCGCCAGGGTTTTCCCAGTCACGACG | |
| | AATTCGATCCGGCTCAAGCTTTTGGACCCTCGTACAGAAGCTAAT | |
| | ACGACTCACTATA | |
| 20 | GGGAAATAAGAGAGAAAAGAAGAGTAAGAAGAAATATAAGAGCCA | Plasmid |
| | CCATGGGAGTGCACGAGTGTCCCGCGTGGTTGTGGTTGCTGCTGT | 2 |
| | CGCTCTTGAGCCTCCCACTGGGACTGCCTGTGCTGGGGGCACCAC | (with |
| | CCAGATTGATCTGCGACTCACGGGTACTTGAGAGGTACCTTCTTG | EPO |
| | AAGCCAAAGAAGCCGAAAACATCACAACCGGATGCGCCGAGCACT | as |
| | GCTCCCTCAATGAGAACATTACTGTACCGGATACAAAGGTCAATT | ORF, |
| | TCTATGCATGGAAGAGAATGGAAGTAGGACAGCAGGCCGTCGAAG | which |
| | TGTGGCAGGGGCTCGCGCTTTTGTCGGAGGCGGTGTTGCGGGGTC | can |
| | AGGCCCTCCTCGTCAACTCATCACAGCCGTGGGAGCCCCTCCAAC | be |
| | TTCATGTCGATAAAGCGGTGTCGGGGCTCCGCAGCTTGACGACGT | removed) |
| | TGCTTCGGGCTCTGGGCGCACAAAAGGAGGCTATTTCGCCGCCTG | |
| | ACGCGGCCTCCGCGGCACCCCTCCGAACGATCACCGCGGACACGT | |
| | TTAGGAAGCTTTTTAGAGTGTACAGCAATTTCCTCCGCGGAAAGC | |
| | TGAAATTGTATACTGGTGAAGCGTGTAGGACAGGGGATCGCTGAT | |
| | AATAGGCTGGAGCCTCGGTGGCCATGCTTCTTGCCCCTTGGGCCT | |
| | CCCCCCAGCCCCTCCTCCCCTTCCTGCACCCGTACCCCCGTGGTC | |
| | TTTGAATAAAGTCTGAGTGGGCGGCAAAAAAAAAAAAAAAAAAAA | |
| | AAAAAAAAAAAAAAAAAAAAAAAAAAAAAAAAAAAAAAAAAAAAA | |
| | AAAAAAAAAAAAAAAAAAAAAAAAAAAAAAAAAAATCTAGACATC | |
| | CCTTCAGAGTCCCGGGTAGCATAACCCCTTGGGGCCTCTAAACGG | |
| | GTCTTGAGGGGTTTTTTGCGAGCTCGGTACCCAGCCCCGACGAGC | |
| | TTCATGCCGTTAGTCGCACTGCAAGGGGTGTTATGAGCCATATTC | |
| | AGGTATAAATGGGCTCGCGATAATGTTCAGAATTGGTTAATTGGT | |
| | TGTAACACTGACCCCTATTTGTTTATTTTTCTAAATACATTCAAA | |
| | TATGTATCCGCTCATGAGACAATAACCCTGATAAATGCTTCAATA | |
| | ATATTGAAAAAGGAAGAATATGAGCCATATTCAACGGGAAACGTC | |
| | GAGGCCGCGATTAAATTCCAACATGGACGCTGATTTATATGGGTA | |
| | TAAATGGGCTCGCGATAATGTCGGGCAATCAGGTGCGACAATCTA | |
| | TCGCTTGTATGGGAAGCCCGATGCGCCAGAGTTGTTTCTGAAACA | |
| | TGGCAAAGGTAGCGTTGCCAATGATGTTACAGATGAGATGGTCAG | |
| | ACTAAACTGGCTGACGGAATTTATGCCACTTCCGACCATCAAGCA | |
| | TTTTATCCGTACTCCTGATGATGCATGGTTACTCACCACTGCGAT | |
| | CCCCGGAAAAACAGCGTTCCAGGTATTAGAAGAATATCCTGATTC | |
| | AGGTGAAAATATTGTTGATGCGCTGGCAGTGTTCCTGCGCCGGTT | |
| | GCACTCGATTCCTGTTTGTAATTGTCCTTTTAACAGCGATCGCGT | |
| | CTTCCGTCTTGCACAAGCGCAATCACGAATGAATAACGGTTTGGT | |
| | TGATGCGAGTGATTTTGATGACGAGCGTAATGGCTGGCCTGTTGA | |
| | ACAAGTCTGGAAAGAAATGCATAAACTTTTGCCATTCTCACCGGA | |
| | TTCAGTCGTCACTCATGGTGATTTCTCACTTGATAACCTTATTTT | |
| | TGACGAGGGGAAATTAATAGGTTGTATTGATGTTGGACGAGTCGG | |
| | AATCGCAGACCGATACCAGGATCTTGCCATTCTATGGAACTGCCT | |
| | CGGTGAGTTTTCTCCTTCATTACAGAAACGGCTTTTTCAAAAATA | |
| | TGGTATTGATAATCCTGATATGAATAAATTGCAGTTTCATTTGAT | |
| | GCTCGATGAGTTTTTCTAAGCAGAGCATTACGCTGACTTGACGGG | |
| | ACGGCGCAAGCTCATGACCAAAATCCCTTAACGTGAGTTACGCGC | |
| | GCGTCGTTCCACTGAGCGTCAGACCCCGTAGAAAAGATCAAAGGA | |
| | TCTTCTTGAGATCCTTTTTTTCTGCGCGTAATCTGCTGCTTGCAA | |
| | ACAAAAAAACCACCGCTACCAGCGGTGGTTTGTTTGCCGGATCAA | |
| | GAGCTACCAACTCTTTTTCCGAAGGTAACTGGCTTCAGCAGAGCG | |
| | CAGATACCAAATACTGTTCTTCTAGTGTAGCCGTAGTTAGCCCAC | |
| | CACTTCAAGAACTCTGTAGCACCGCCTACATACCTCGCTCTGCTA | |
| | ATCCTGTTACCAGTGGCTGCTGCCAGTGGCGATAAGTCGTGTCTT | |
| | ACCGGGTTGGACTCAAGACGATAGTTACCGGATAAGGCGCAGCGG | |
| | TCGGGCTGAACGGGGGGTTCGTGCACACAGCCCAGCTTGGAGCGA | |
| | ACGACCTACACCGAACTGAGATACCTACAGCGTGAGCTATGAGAA | |
| | AGCGCCACGCTTCCCGAAGGGAGAAAGGCGGACAGGTATCCGGTA | |
| | AGCGGCAGGGTCGGAACAGGAGAGCGCACGAGGGAGCTTCCAGGG | |
| | GGAAACGCCTGGTATCTTTATAGTCCTGTCGGGTTTCGCCACCTC | |
| | TGACTTGAGCGTCGATTTTTGTGATGCTCGTCAGGGGGGCGGAGC | |
| | CTATGGAAAAACGCCAGCAACGCGGCCTTTTTACGGTTCCTGGCC | |
| | TTTTGCTGGCCTTTTGCTCACATGTTCTTTCCTGCGTTATCCCCT | |
| | GATTCTGTGGATAACCGTATTACCGCCTGCTCGCCGCAGCCGAAC | |
| | GACCGAGCGCAGCGAGTCAGTGAGCGAGGAAGCGGAAGGCGAGAG | |
| | TAGGGAACTGCCAGGCATCAAACTAAGCAGAAGGCCCCTGACGCA | |
| | TGGCCTTTTTGCGTTTCTACAAACTCTTTCTGTGTTGTAAAACGA | |
| | CGGCCAGTCTTAAGCTCGGGCCCCTTTTCCGCCAGGGTTTTCCCA | |
| | GTCACGACGAATTCGATCCGGCAATCTAGAAATCAAGCTTTTGGA | |
| | CCCTCGTACAGAAGCTAATACGACTCACTATA | |
| 21 | gcagagcattacgctgacttgacgggacggcgcaagctcatgacc | pStrain7 |
| | aaaatcccttaacgtgagttacgcgcgcgcttatgttttcgctga | (full |
| | tatcccgagcggtttcaaaattgtgatctatatttaacaagcaaa | plasmid |
| | caaaaaaaccaccgctaccagcggtggtttgtttgccggatcaag | including |
| | agctaccaactctttttccgaaggtaactggcttcagcagagcgc | insert |
| | agataccaaatactgttcttctagtgtagccgtagttagcccacc | and |
| | acttcaagaactctgtagcaccgcctacatacctcgctctgctaa | poly-A |
| | tcctgttaccagtggctgctgccagtggcgataagtcgtgtctta | tail) |
| | ccgggttggactcaagacgatagttaccggataaggcgcagcggt | |
| | cgggctgaacggggggttcgtgcacacagcccagcttggagcgaa | |
| | cgacctacaccgaactgagatacctacagcgtgagctatgagaaa | |
| | gcgccacgcttcccgaagggagaaaggcggacaggtatccggtaa | |
| | gcggcagggtcggaacaggagagcgcacgagggagcttccagggg | |
| | gaaacgcctggtatctttatagtcctgtcgggtttcgccacctct | |
| | gacttgagcgtcgatttttgtgatgctcgtcaggggggcggagcc | |
| | tatggaaaaacgccagcaacgcggcctttttacggttcctggcct | |
| | tttgctggccttttgctcacatgttctttcctgcgttatcccctg | |
| | attctgtggataaccgtattaccgcctttgagtgagctgataccg | |
| | ctcgccgcagccgaacgaccgagcgtatggtgcactctcagtaca | |
| | atctgctctgatgccgcatagttaagccagtatacactccgctat | |
| | cgctacgtgactgggtcatggctgcgccccgacacccgccaacac | |
| | ccgctgacgcgccctgacgggcttgtctgctcccggcatccgctt | |
| | acagacaagctgtgaccgtctccgggagctgctgccaggcatcaa | |
| | actaagcagaaggcccctgacgcatggcctttttgcgtttctacA | |
| | AACTCTTTCTGTGTTGTAAAACGACGGCCAGTCTTAAGCTCGGGC | |
| | CCCTTTTCCGCCAGGGTTTTCCCAGTCACGACGAATTCGATCCGG | |
| | Ctcaagcttttggaccctcgtacagaagctaatacgactcactat | |
| | agggaaataagagagaaaagaagagtaagaagaaatataagagcc | |
| | accatggaagatgcgaagaacatcaagaagggacctgccccgttt | |
| | taccctttggaggacggtacagcaggagaacagctccacaaggcg | |
| | atgaaacgctacgccctggtccccggaacgattgcgtttaccgat | |
| | gcacatattgaggtagacatcacatacgcagaatacttcgaaatg | |
| | tcggtgaggctggcggaagcgatgaagagatatggtcttaacact | |
| | aatcaccgcatcgtggtgtgttcggagaactcattgcagtttttc | |
| | atgccggtccttggagcacttttcatcggggtcgcagtcgcgcca | |
| | gcgaacgacatctacaatgagcgggaactcttgaatagcatggga | |
| | atctcccagccgacggtcgtgtttgtctccaaaaaggggctgcag | |
| | aaaatcctcaacgtgcagaagaagctccccattattcaaaagatc | |
| | atcattatggatagcaagacagattaccaagggttccagtcgatg | |
| | tatacctttgtgacatcgcatttgccgccagggtttaacgagtat | |
| | gacttcgtccccgagtcatttgacagagataaaaccatcgcgctg | |
| | attatgaattcctcgggtagcaccggtttgccaaagggggtggcg | |
| | ttgccccaccgcactgcttgtgtgcggttctcgcacgctagggat | |
| | cctatctttggtaatcagatcattcccgacacagcaatcctgtcc | |
| | gtggtaccttttcatcacggttttggcatgttcacgactctcggc | |
| | tatttgatttgcggtttcagggtcgtacttatgtatcggttcgag | |
| | gaagaactgtttttgagatccttgcaagattacaagatccagtcg | |
| | gccctccttgtgccaacgcttttctcattctttgcgaaatcgaca | |
| | cttattgataagtatgacctttccaatctgcatgagattgcctca | |
| | gggggagcgccgcttagcaaggaagtcggggaggcagtggccaag | |
| | cgcttccaccttcccggaattcggcagggatacgggctcacggag | |
| | acaacatccgcgatccttatcacgcccgagggtgacgataagccg | |
| | ggagccgtcggaaaagtggtccccttctttgaagccaaggtcgta | |
| | gacctcgacacgggaaaaaccctcggagtgaaccagaggggcgag | |
| | ctctgcgtgagagggccgatgatcatgtcaggttacgtgaataac | |
| | cctgaagcgacgaatgcgctgatcgacaaggatgggtggttgcat | |
| | tcgggagacattgcctattgggatgaggatgagcacttctttatc | |
| | gtagatcgacttaagagcttgatcaaatacaaaggctatcaggta | |
| | gcgcctgccgagctcgagtcaatcctgctccagcaccccaacatt | |
| | ttcgacgccggagtggccgggttgcccgatgacgacgcgggtgag | |
| | ctgccagcggccgtggtagtcctcgaacatgggaaaacaatgacc | |
| | gaaaaggagatcgtggactacgtagcatcacaagtgacgactgcg | |
| | aagaaactgaggggaggggtagtctttgtggacgaggtcccgaaa | |
| | ggcttgactgggaagcttgacgctcgcaaaatccgggaaatcctg | |
| | attaaggcaaagaaaggcgggaaaatcgctgtctgataataggct | |
| | ggagcctcggtggccatgcttcttgccccttgggcctccccccag | |
| | cccctcctccccttcctgcacccgtacccccgtggtctttgaata | |
| | aagtctgagtgggcggcaaaaaaaaaaaaaaaaaaaaaaaaaaaa | |
| | aaaaaaaaaaaaaaaaaaaaaaaaaaaaaaaaaaaaaaaaaaaaa | |
| | aaaaaaaaaaaaaaaaaaaaaaaaaaatctagacatcccttcaga | |
| | gtcccgggtagcataaccccttggggcctctaaacgggtcttgag | |
| | gggttttttgcgagctcggtacccagccccgacgagcttcatgcc | |
| | gttagtcgcactgcaaggggtgttatgagccatattcaggtataa | |
| | atgggctcgcgataatgttcagaattggttaattggttgtaacac | |
| | tgacccctatttgtttatttttctaaatacattcaaatatgtatc | |
| | cgctcatgagacaataaccctgataaatgcttcaataatattgaa | |
| | aaaggaagaatatgagccatattcaacgggaaacgtcgaggccgc | |
| | gattaaattccaacatggacgctgatttatatgggtataaatggg | |
| | ctcgcgataatgtcgggcaatcaggtgcgacaatctatcgcttgt | |
| | atgggaagcccgatgcgccagagttgtttctgaaacatggcaaag | |
| | gtagcgttgccaatgatgttacagatgagatggtcagactaaact | |
| | ggctgacggaatttatgccacttccgaccatcaagcattttatcc | |
| | gtactcctgatgatgcatggttactcaccactgcgatccccggaa | |
| | aaacagcgttccaggtattagaagaatatcctgattcaggtgaaa | |
| | atattgttgatgcgctggcagtgttcctgcgccggttgcactcga | |
| | ttcctgtttgtaattgtccttttaacagcgatcgcgtcttccgtc | |
| | ttgcacaagcgcaatcacgaatgaataacggtttggttgatgcga | |
| | gtgattttgatgacgagcgtaatggctggcctgttgaacaagtct | |
| | ggaaagaaatgcataaacttttgccattctcaccggattcagtcg | |
| | tcactcatggtgatttctcacttgataaccttatttttgacgagg | |
| | ggaaattaataggttgtattgatgttggacgagtcggaatcgcag | |
| | accgataccaggatcttgccattctatggaactgcctcggtgagt | |
| | tttctccttcattacagaaacggctttttcaaaaatatggtattg | |
| | ataatcctgatatgaataaattgcagtttcatttgatgctcgatg | |
| | agtttttctaa | |
| 22 | cttctttgaagccaaggtcgtagacctcgacacgggaaaaaccct | pStrain8 |
| | cggagtgaaccagaggggcgagctctgcgtgagagggccgatgat | (full |
| | catgtcaggttacgtgaataaccctgaagcgacgaatgcgctgat | plasmid |
| | cgacaaggatgggtggttgcattcgggagacattgcctattggga | including |
| | tgaggatgagcacttctttatcgtagatcgacttaagagcttgat | insert |
| | caaatacaaaggctatcaggtagcgcctgccgagctcgagtcaat | and |
| | cctgctccagcaccccaacattttcgacgccggagtggccgggtt | poly-A |
| | gcccgatgacgacgcgggtgagctgccagcggccgtggtagtcct | tail) |
| | cgaacatgggaaaacaatgaccgaaaaggagatcgtggactacgt | |
| | agcatcacaagtgacgactgcgaagaaactgaggggaggggtagt | |
| | ctttgtggacgaggtcccgaaaggcttgactgggaagcttgacgc | |
| | tcgcaaaatccgggaaatcctgattaaggcaaagaaaggcgggaa | |
| | aatcgctgtctgataataggctggagcctcggtggccatgcttct | |
| | tgccccttgggcctccccccagcccctcctccccttcctgcaccc | |
| | gtacccccgtggtctttgaataaagtctgagtgggcggcaaaaaa | |
| | aaaaaaaaaaaaaaaaaaaaaaaaaaaaaaaaaaaaaaaaaaaaa | |
| | aaaaaaaaaaaaaaaaaaaaaaaaaaaaaaaaaaaaaaaaaaaaa | |
| | aaaatctagacatcccttcagagtcccgggtagcataaccccttg | |
| | gggcctctaaacgggtcttgaggggttttttgcgagctcggtacc | |
| | cagccccgacgagcttcatgccgttagtcgcactgcaaggggtgt | |
| | tatgagccatattcaggtataaatgggctcgcgataatgttcaga | |
| | attggttaattggttgtaacactgacccctatttgtttatttttc | |
| | taaatacattcaaatatgtatccgctcatgagacaataaccctga | |
| | taaatgcttcaataatattgaaaaaggaagaatatgagccatatt | |
| | caacgggaaacgtcgaggccgcgattaaattccaacatggacgct | |
| | gatttatatgggtataaatgggctcgcgataatgtcgggcaatca | |
| | ggtgcgacaatctatcgcttgtatgggaagcccgatgcgccagag | |
| | ttgtttctgaaacatggcaaaggtagcgttgccaatgatgttaca | |
| | gatgagatggtcagactaaactggctgacggaatttatgccactt | |
| | ccgaccatcaagcattttatccgtactcctgatgatgcatggtta | |
| | ctcaccactgcgatccccggaaaaacagcgttccaggtattagaa | |
| | gaatatcctgattcaggtgaaatacattcaaatatgtatccgctc | |
| | atgagacaataaccctgataaatgcttcaataatattgaaaaagg | |
| | aagaatatgagccatattcaacgggaaacgtcgaggccgcgatta | |
| | aattccaacatggacgctgatttatatgggtataaatgggctcgc | |
| | gataatgtcgggcaatcaggtgcgacaatctatcgcttgtatggg | |
| | aagcccgatgcgccagagttgtttctgaaacatggcaaaggtagc | |
| | gttgccaatgatgttacagatgagatggtcagactaaactggctg | |
| | acggaatttatgccacttccgaccatcaagcattttatccgtact | |
| | cctgatgatgcatggttactcaccactgcgatccccggaaaaaca | |
| | gcgttccaggtattagaagaatatcctgattcaggtgaaaatatt | |
| | gttgatgcgctggcagtgttcctgcgccggttgcactcgattcct | |
| | gtttgtaattgtccttttaacagcgatcgcgtcttccgtcttgca | |
| | caagcgcaatcacgaatgaataacggtttggttgatgcgagtgat | |
| | tttgatgacgagcgtaatggctggcctgttgaacaagtctggaaa | |
| | gaaatgcataaacttttgccattctcaccggattcagtcgtcact | |
| | catggtgatttctcacttgataaccttatttttgacgaggggaaa | |
| | ttaataggttgtattgatgttggacgagtcggaatcgcagaccga | |
| | taccaggatcttgccattctatggaactgcctcggtgagttttct | |
| | ccttcattacagaaacggctttttcaaaaatatggtattgataat | |
| | cctgatatgaataaattgcagtttcatttgatgctcgatgagttt | |
| | ttctaa | |
| 23 | ATGCCAGACATGAAGCTGTTTGCGGGTAACGCGACCCCTGAGCTG | prsA* |
| | GCCCAGCGTATCGCGAACCGCTTGTACACGAGCCTGGGTGACGCA | sequence |
| | GCGGTTGGCCGTTTCAGCGATGGTGAAGTCAGCGTGCAGATTAAT | (ORF |
| | GAAAATGTGCGTGGTGGCGACATTTTCATCATTCAGAGCACCTGT | only) |
| | GCGCCGACGAACGATAACCTGATGGAATTGGTTGTGATGGTCGAT | |
| | GCACTGCGTCGCGCCTCCGCCGGTCGCATTACCGCGGTGATTCCG | |
| | TATTTTGGCTATGCACGCCAGGATCGTCGTGTCCGCTCCGCGCGC | |
| | GTCCCGATCACGGCGAAAGTCGTCGCGGATTTTCTGAGCAGCGTG | |
| | GGTGTTGACCGTGTGCTGACCGTGGCGTTGCATGCTGAGCAAATT | |
| | CAAGGTTTCTTCGACGTCCCGGTGGATAATGTTTTCGGTTCTCCG | |
| | ATTCTTCTGGAAGATATGCTGCAACTGAATCTGGATAATCCGATC | |
| | GTCGTTAGCCCGGATATCGGTGGCGTGGTGCGTGCGCGTGCAATT | |
| | GCAAAGCTGCTGAATGATACCGACATGGCAATCATCGACAAGCGC | |
| | CGTCCGCGTGCGAATGTCAGCCAAGTCATGCACATCATTGGCGAC | |
| | GTTGCTGGCCGTGACTGCGTTTTAGTGGACGACATGATCGATACG | |
| | GGTGGCACTCTGTGTAAAGCCGCTGAGGCCCTGAAAGAGCGCGGT | |
| | GCGAAACGTGTTTTCGCATACGCGACGCACCCGATCTTTAGCGGT | |
| | AATGCTGCGAACAACTTGCGTAACTCTGTTATTGACGAAGTTGTT | |
| | GTTTGCGACACCATTCCGCTGAGCGACGAAATCAAGAGCCTGCCG | |
| | AACGTGCGTACCCTGACCCTGAGCGGCATGCTCGCAGAGGCCATC | |
| | AGACGTATTAGCAACGAAGAGTCGATCAGCGCGATGTTTGAGCAT | |
| | TGA | |
| 24 | MPDMKLFAGNATPCLAQRIANRLYTSLGDAAVGRFSDGCVSVQIN | prsA* |
| | CNVRGGDIFIIQSTCAPTNDNLMCLVVMVDALRRASAGRITAVIP | sequence |
| | YFGYARQDRRVRSARVPITAKVVADFLSSVGVDRVLTVALHACQI | (amino |
| | QGFFDVPVDNVFGSPILLCDMLQLNLDNPIVVSPDIGGVVRARAI | acid |
| | AKLLNDTDMAIIDKRRPRANVSQVMHIIGDVAGRDCVLVDDMIDT | sequence- |
| | GGTLCKAACALKCRGAKRVFAYATHPIFSGNAANNLRNSVIDCVV | Key |
| | VCDTIPLSDCIKSLPNVRTLTLSGMLACAIRRISNCCSISAMFCH | mutation |
| | | in |
| | | underline) |
| 25 | cggcgtaccgcaacacttttgttgtgcgtaaggtgtg | PurR |
| | taaaggcaaacgtttaccttgcgattttgcaggagctga | locus |
| | agttagggtctggagtgaaatgga | in |
| | atggcaacaataaaagatgtagcgaaacgagcaaa | wildtype |
| | cgtttccactacaactgtgtcacacg | E coli |
| | tgatcaacaaaacacgtttcgtcgctgaagaaacgcgcaacgcc | MG1655 = |
| | gtgtgggcagcgattaaagaattacactactcccctagc | (sequence |
| | gcggtggcgcgtagcctgaaggttaaccacaccaagtctatcg | upstream: |
| | gtttgctggcgaccagcagcgaagcggcctattttgccg | single |
| | agatcattgaagcagttgaaaaaaattgcttccagaaaggttac | underline; |
| | accctgattctgggcaatgcgtggaacaatcttgagaaac | downstream |
| | agcgggcttatctgtcgatgatggcgcaaaaacgcgtcgatggtct | of |
| | gctggtgatgtgttctgagtacccagagccgttgctggcgatg | ORF: |
| | ctggaagagtatcgccatatcccaatggtggtcatggactgg | italics; |
| | ggtgaagcaaaagctgacttcaccgatgcggtcatt | and |
| | gataacgcgttcgaaggcggctacatggccgggcgttatctgattgaa | ORF |
| | cgcggtcaccgcgaaatcggcgtcatccccggc | in |
| | ccgctggaacgtaacaccggcgcaggccgccttgccggttttatga | double |
| | aggcgatggaagaagcgatgatcaaggtgccgga | underline) |
| | aagctggattgtgcagggtgactttgaacctgaatccggttatcgc | |
| | gccatgcagcaaatcctgtcgcagccgcatcgccctactgc | |
| | cgtcttctgtggtggcgatatcatggcaatgggcgcactttgt | |
| | gctgctgatgaaatgggcctgcgcgtcccgcaggatgtttc | |
| | gctgatcggttatgataacgtgcgcaacgcgcgctattttacgcc | |
| | ggcgctgaccacgatccatcagccaaaagattcgctgggt | |
| | gaaacagcgttcaacatgctgttggatcgtatcgtcaacaaacg | |
| | tgaagaaccgcagtctattgaagtgcatccgcgct | |
| | tgattgaacgccgctccgtggctgacggcccgttccgcgac | |
| | tatcgtcgttaa | |
| | tcacccgttgcgggagtctcttcttccggctccc | |
| | gcagccactccttattcagcgtctcactatcgccgagat | |
| | actcaagcaaccaggttaacgcaggcgaca | |
| 26 | catcgatttattaagacccactttcacatttaagttgtttttcta | \|<repA101 |
| | atccgcatatgatcaattcaaggccgaataagaaggctggctctg | \|ori1 |
| | caccttggtgatcaaataattcgatagcttgtcgtaataatggcg | 01_ts\|<recA\|<b |
| | gcatactatcagtagtaggtgtttccctttcttctttagcgactt | la\|<tetR\|<P(tet |
| | gatgctcttgatcttccaatacgcaacctaaagtaaaatgcccca | R)\|P(tet)>\|gam |
| | cagcgctgagtgcatataatgcattctctagtgaaaaaccttgtt | ma>\|beta>\|exo |
| | ggcataaaaaggctaattgattttcgagagtttcatactgttttt | >\|60a>\|) |
| | ctgtaggccgtgtacctaaatgtacttttgctccatcgcgatgac | |
| | ttagtaaagcacatctaaaacttttagcgttattacgtaaaaaat | |
| | cttgccagctttccccttctaaagggcaaaagtgagtatggtgcc | |
| | tatctaacatctcaatggctaaggcgtcgagcaaagcccgcttat | |
| | tttttacatgccaatacaatgtaggctgctctacacctagcttct | |
| | gggcgagtttacgggttgttaaaccttcgattccgacctcattaa | |
| | gcagctctaatgcgctgttaatcactttacttttatctaatctag | |
| | acatcattaattcctaattgctagcattgtacctaggactgagct | |
| | agccataaagttgacactctatcgttgatagagttattttaccac | |
| | tccctatcagtgatagagaaagaattcaaaggatccaaacaggag | |
| | acattaaatggatattaatactgaaactgagatcaagcaaaagca | |
| | ttcactaaccccctttcctgttttcctaatcagcccggcatttcg | |
| | cgggcgatattttcacagctatttcaggagttcagccatgaacgc | |
| | ttattacattcaggatcgtcttgaggctcagagctgggcgcgtca | |
| | ctaccagcagctcgcccgtgaagagaaagaggcagaactggcaga | |
| | cgacatggaaaaaggcctgccccagcacctgtttgaatcgctatg | |
| | catcgatcatttgcaacgccacggggccagcaaaaaatccattac | |
| | ccgtgcgtttgatgacgatgttgagtttcaggagcgcatggcaga | |
| | acacatccggtacatggttgaaaccattgctcaccaccaggttga | |
| | tattgattcagaggtataaaacgaatgagtactgcactcgcaacg | |
| | ctggctgggaagctggctgaacgtgtcggcatggattctgtcgac | |
| | ccacaggaactgatcaccactcttcgccagacggcatttaaaggt | |
| | gatgccagcgatgcgcagttcatcgcattactgatcgttgccaac | |
| | cagtacggccttaatccgtggacgaaagaaatttacgcctttcct | |
| | gataagcagaatggcatcgttccggtggtgggcgttgatggctgg | |
| | tcccgcatcatcaatgaaaaccagcagtttgatggcatggacttt | |
| | gagcaggacaatgaatcctgtacatgccggatttaccgcaaggac | |
| | cgtaatcatccgatctgcgttaccgaatggatggatgaatgccgc | |
| | cgcgaaccattcaaaactcgcgaaggcagagaaatcacggggccg | |
| | tggcagtcgcatcccaaacggatgttacgtcataaagccatgatt | |
| | cagtgtgcccgtctggccttcggatttgctggtatctatgacaag | |
| | gatgaagccgagcgcattgtcgaaaatactgcatacactgcagaa | |
| | cgtcagccggaacgcgacatcactccggttaacgatgaaaccatg | |
| | caggagattaacactctgctgatcgccctggataaaacatgggat | |
| | gacgacttattgccgctctgttcccagatatttcgccgcgacatt | |
| | cgtgcatcgtcagaactgacacaggccgaagcagtaaaagctctt | |
| | ggattcctgaaacagaaagccgcagagcagaaggtggcagcatga | |
| | caccggacattatcctgcagcgtaccgggatcgatgtgagagctg | |
| | tcgaacagggaagaagtggcctgacatgaaaatgtcctacttcca | |
| | caccctgcttgctgaggtttgcaccggtgtggctccggaagttaa | |
| | cgctaaagcactggcctggggaaaacagtacgagaacgacgccag | |
| | aaccctgtttgaattcacttccggcgtgaatgttactgaatcccc | |
| | gatcatctatcgcgacgaaagtatgcgtaccgcctgctctcccga | |
| | tggtttatgcagtgacggcaacggccttgaactgaaatgcccgtt | |
| | tacctcccgggatttcatgaagttccggctcggtggtttcgaggc | |
| | cataaagtcagcttacatggcccaggtgcagtacagcatgtgggt | |
| | gacgcgaaaaaatgcctggtactttgccaactatgacccgcgtat | |
| | gaagcgtgaaggcctgcattatgtcgtgattgagcgggatgaaaa | |
| | gtacatggcgagttttgacgagatcgtgccggagttcatcgaaaa | |
| | aatggacgaggcactggctgaaattggttttgtatttggggagca | |
| | atggcgatgacgcatcctcacgataatatccgggtaggcgcaatc | |
| | actttcgtctactccgttacaaagcgaggctgggtatttcccggc | |
| | ctttctgttatccgaaatccactgaaagcacagcggctggctgag | |
| | gagataaataataaacgaggggctgtatgcacaaagcatcttctg | |
| | ttgagttaagaacgagtatcgagatggcacatagccttgctcaaa | |
| | ttggaatcaggtttgtgccaataccagtagaaacagacgaagaat | |
| | ccatgggtatggacagttttccctttgatatgtaacggtgaacag | |
| | ttgttctacttttgtttgttagtcttgatgcttcactgatagata | |
| | caagagccataagaacctcagatccttccgtatttagccagtatg | |
| | ttctctagtgtggttcgttgtttttgcgtgagccatgagaacgaa | |
| | ccattgagatcatacttactttgcatgtcactcaaaaattttgcc | |
| | tcaaaactggtgagctgaatttttgcagttaaagcatcgtgtagt | |
| | gtttttcttagtccgttaTgtaggtaggaatctgatgtaatggtt | |
| | gttggtattttgtcaccattcatttttatctggttgttctcaagt | |
| | tcggttacgagatccatttgtctatctagttcaacttggaaaatc | |
| | aacgtatcagtcgggcggcctcgcttatcaaccaccaatttcata | |
| | ttgctgtaagtgtttaaatctttacttattggtttcaaaacccat | |
| | tggttaagccttttaaactcatggtagttattttcaagcattaac | |
| | atgaacttaaattcatcaaggctaatctctatatttgccttgtga | |
| | gttttcttttgtgttagttcttttaataaccactcataaatcctc | |
| | atagagtatttgttttcaaaagacttaacatgttccagattatat | |
| | tttatgaatttttttaactggaaaagataaggcaatatctcttca | |
| | ctaaaaactaattctaatttttcgcttgagaacttggcatagttt | |
| | gtccactggaaaatctcaaagcctttaaccaaaggattcctgatt | |
| | tccacagttctcgtcatcagctctctggttgctttagctaataca | |
| | ccataagcattttccctactgatgttcatcatctgaAcgtattgg | |
| | ttataagtgaacgataccgtccgttctttccttgtagggttttca | |
| | atcgtggggttgagtagtgccacacagcataaaattagcttggtt | |
| | tcatgctccgttaagtcatagcgactaatcgctagttcatttgct | |
| | ttgaaaacaactaattcagacatacatctcaattggtctaggtga | |
| | ttttaatcactataccaattgagatgggctagtcaatgataatta | |
| | ctagtccttttcctttgagttgtgggtatctgtaaattctgctag | |
| | acctttgctggaaaacttgtaaattctgctagaccctctgtaaat | |
| | tccgctagacctttgtgtgttttttttgtttatattcaagtggtt | |
| | ataatttatagaataaagaaagaataaaaaaagataaaaagaata | |
| | gatcccagccctgtgtataactcactactttagtcagttccgcag | |
| | tattacaaaaggatgtcgcaaacggcaaatcgctgaatattcctt | |
| | ttgtctccgaccatcaggcacctgagtcgctgtctttttcgtgac | |
| | attcagttcgctgcgctcacggctctggcagtgaatgggggtaaa | |
| | tggcactacaggcgccttttatggattcatgcaaggaaactaccc | |
| | ataatacaagaaaagcccgtcacgggcttctcagggcgttttatg | |
| | ggggtctgctatgtggtgctatctgactttttgctgttcagcagt | |
| | tcctgccctctgattttccagtctgaccacttcggattatcccgt | |
| | gacaggtcattcagactggctaatgcacccagtaaggcagcggta | |
| | tcatcaacggggtctgacgctcagtggaacgaaaactcacgttaa | |
| | gggattttggtcatgagattatcaaaaaggatcttcacctagatc | |
| | cttttaaattaaaaatgaagttttaaatcaatctaaagtatatat | |
| | gagtaaacttggtctgacagttagaaatcttcgttggtctcggct | |
| | ctccttcgctatcgtccacgctgaagtccggcgtactattagggt | |
| | tgctcagtaacagctcacgcactttcttctcaatctctttggctg | |
| | tctcgggattgtccttcagccatgccgtggcattagctttgcctt | |
| | ggccaatcttctctcctttgtaagagtaccaggcaccagcctttt | |
| | caatcagcttttctttcacccccaaatctaccaattcgccgtaga | |
| | aattaattccttctccatataaaatctggaactccgcctgcttaa | |
| | acggggctgcgattttattctttactactttgacgcgggtctcac | |
| | tcccgacgacgttttcaccctctttgactgccccgatgcgacgaa | |
| | tatccaaacgaacggacgcatagaacttcagcgcattacctccgg | |
| | tagtggtttcaggattgccaaacataactccaatcttcatgcgga | |
| | tttgattgataaagataagaagcgtgttggattgcttcaggttcc | |
| | ccgccaacttgcgcattgcctggctcatcatgcgggcagcaagcc | |
| | ccatgtgactgtcaccaatctcgccctcgatctcagctttaggcg | |
| | tcagagcggccactggagcataaaagattgtcaatatcgaccccc | |
| | aacttgcgcgcatagatcggatcaagcgcgtgttctgcgtcgata | |
| | aaagcacaatacgacccataggtaaaccgcctgcacctaaggcaa | |
| | tatccagcgaaagagacccggtgctgatggtctctacatccatag | |
| | agcggtcctcccccaagcgcataatagatcccttgccaaattgtt | |
| | tctcgatttggcccagcgcggcggccaatgctttctgtttatttt | |
| | catcgattgccatgattgatatcctttttactcctgtcatgtgtg | |
| | aaattgttatccgctcacaattccacacaacatacgagccggaag | |
| | cataaagtgtaaagcctggggtgcctaatgagtgagctaactcac | |
| | attaattgcgttgcgctcactgcccgctttccagtcgggaaacct | |
| | gtcgtgccagctgcattaatgaatcggccaacgcgcggggagagg | |
| | cggtttgcgtattgttaccaatgcttaatcagtgaggcacctatc | |
| | tcagcgatctgtctatttcgttcatccatagttgcctgactcccc | |
| | gtcgtgtagataactacgatacgggagggcttaccatctggcccc | |
| | agtgctgcaatgataccgcgagacccacgctcaccggctccagat | |
| | ttatcagcaataaaccagccagccggaagggccgagcgcagaagt | |
| | ggtcctgcaactttatccgcctccatccagtctattaattgttgc | |
| | cgggaagctagagtaagtagttcgccagttaatagtttgcgcaac | |
| | gttgttgccattgctacaggcatcgtggtgtcacgctcgtcgttt | |
| | ggtatggcttcattcagctccggttcccaacgatcaaggcgagtt | |
| | acatgatcccccatgttgtgcaaaaaagcggttagctccttcggt | |
| | cctccgatcgttgtcagaagtaagttggccgcagtgttatcactc | |
| | atggttatggcagcactgcataattctcttactgtcatgccatcc | |
| | gtaagatgcttttctgtgactggtgagtactcaaccaagtcattc | |
| | tgagaatagtgtatgcggcgaccgagttgctcttgcccggcgtca | |
| | atacgggataataccgcgccacatagcagaactttaaaagtgctc | |
| | atcattggaaaacgttcttcggggcgaaaactctcaaggatctta | |
| | ccgctgttgagatccagttcgatgtaacccactcgtgcacccaac | |
| | tgatcttcagcatcttttactttcaccagcgtttctgggtgagca | |
| | aaaacaggaaggcaaaatgccgcaaaaaagggaataagggcgaca | |
| | cggaaatgttgaatactcatactcttcctttttcaatattattga | |
| | agcatttatcagggttattgtctcatgagcggatacatatttgaa | |
| | tgtatttagaaaaataaacaaataggggttccgcgcacatttccc | |
| | cgaaaagtgccacctg | |
| 27 | gcttatgttttcgctgatatcccgagcggtttcaaaattgtgatc | P(osmY) |
| | tatatttaacaa | |
| 28 | tgttctttcctgcgttatcccctgattctgtggataaccgtatta | PAS |
| | ccgcctttgagtgagctgataccgctcgccgcagccgaacgaccg | |
| | agcgcagcgagtcagtgagcgaggaagcggaagagcgcctgatgc | |
| | ggtattttctccttacgcatctgtgcggtatttcacaccgcatat | |
| | ggtgcactctcagtacaatctgctctgatgccgcatagttaagcc | |
| | agtatacactccgctatcgctacgtgactgggtcatggctgcgcc | |
| | ccgacacccgccaacacccgctgacgcgccctgacgggcttgtct | |
| | gctcccggcatccgcttacagacaagctgtgaccgtctccgggag | |
| | ctg | |
| 29 | Gcaaacaaaaaaaccaccgctaccagcggtggtttgtttgccgga | SL4 |
| | tcaagagctaccaactctttttccgaaggtaactggctt | mutation |
| | cagcagagcgcagataccaaatactgttcttctagtgtaA | in |
| | ccgGagCAagcccaccacttcaagaactc | origin |
| | tgtagcaccgcctacatacctcgctctgctaatcctgttaccagtg | of |
| | gctgctgccagtggcgataagtcgtgtcttaccgggttggactca | replication |
| | agacgatagttaccggataaggcgcagcggtcgggctgaacgggg | (substi |
| | ggttcgtgcacacagcccagcttggagcgaacgacctacaccgaa | tutions |
| | ctgagatacctacagcgtgagctatgagaaagcgccacgcttccc | highlighted |
| | gaagggagaaaggcggacaggtatccggtaagcggcagggtcgga | in |
| | acaggagagcgcacgagggagcttccagggggaaacgcctggtat | underline |
| | ctttatagtcctgtcgggtttcgccacctctgacttgagcgtcga | and |
| | tttttgtgatgctcgtcaggggggcggagcctatggaaaaacgcc | capitals |
| | agcaacgcggcctttttacggttcctggccttttgctggcctttt | ) |
| | gctca | |
| 30 | MYRYLSIAAVVLSAAFSGPALAEGINSFSQAKAAAVKVHADAPGT | endA-AA |
| | FYCGCKINWQGKKGVVDLQSCGYQVRKNENRASRVEWEHVVPAWQ | (www.uniprot. |
| | FGHQRQCWQDGGRKNCAKDPVYRKMESDMHNLQPSVGEVNGDRGN | org/uniprot/P2 |
| | FMYSQWNGGEGQYGQCAMKVDFKEKAAEPPARARGAIARTYFYMR | 5736) |
| | DQYNLTLSRQQTQLFNAWNKMYPVTDWECERDERIAKVQGNHNPY | |
| | VQRACQARKS | |
| 31 | MAIDENKQKALAAALGQIEKQFGKGSIMRLGEDRSMDVETISTGS | recA |
| | LSLDIALGAGGLPMGRIVEIYGPESSGKTTLTLQVIAAAQREGKT | - |
| | CAFIDAEHALDPIYARKLGVDIDNLLCSQPDTGEQALEICDALAR | AA |
| | SGAVDVIVVDSVAALTPKAEIEGEIGDSHMGLAARMMSQAMRKLA | (www.uniprot. |
| | GNLKQSNTLLIFINQIRMKIGVMFGNPETTTGGNALKFYASVRLD | org/uniprot/PO |
| | IRRIGAVKEGENVVGSETRVKVVKNKIAAPFKQAEFQILYGEGIN | A7G6) |
| | FYGELVDLGVKEKLIEKAGAWYSYKGEKIGQGKANATAWLKDNPE | |
| | TAKEIEKKVRELLLSNPNSTPDFSVDDSEGVAETNEDF | |
| 32 | MTVPTYDKFIEPVLRYLATKPEGAAARDVHEAAADALGLDDSQRA | Multiple |
| | KVITSGQLVYKNRAGWAHDRLKRAGLSQSLSRGKWCLTPAGFDWV | knockouts |
| | ASHPQPMTEQETNHLAFAFVNVKLKSRPDAVDLDPKADSPDHEEL | including |
| | AKSSPDDRLDQALKELRDAVADEVLENLLQVSPSRFEVIVLDVLH | hsdR |
| | RLGYGGHRDDLQRVGGTGDGGIDGVISLDKLGLEKVYVQAKRWQN | and |
| | TVGRPELQAFYGALAGQKAKRGVFITTSGFTSQARDFAQSVEGMV | mrr-AA |
| | LVDGERLVHLMIENEVGVSSRLLKVPKLDMDYFE | |
| 33 | atggcaacaataaaagatgtagcgaaacgagcaaacgtttccact | purR |
| | acaactgtgtcacacgtgatcaacaaaacacgtttcgtcgctgaa | (ORF |
| | gaaacgcgcaacgccgtgtgggcagcgattaaagaattacactac | only) |
| | tcccctagcgcggtggcgcgtagcctgaaggttaaccacaccaag | |
| | tctatcggtttgctggcgaccagcagcgaagcggcctattttgcc | |
| | gagatcattgaagcagttgaaaaaaattgcttccagaaaggttac | |
| | accctgattctgggcaatgcgtggaacaatcttgagaaacagcgg | |
| | gcttatctgtcgatgatggcgcaaaaacgcgtcgatggtctgctg | |
| | gtgatgtgttctgagtacccagagccgttgctggcgatgctggaa | |
| | gagtatcgccatatcccaatggtggtcatggactggggtgaagca | |
| | aaagctgacttcaccgatgcggtcattgataacgcgttcgaaggc | |
| | ggctacatggccgggcgttatctgattgaacgcggtcaccgcgaa | |
| | atcggcgtcatccccggcccgctggaacgtaacaccggcgcaggc | |
| | cgccttgccggttttatgaaggcgatggaagaagcgatgatcaag | |
| | gtgccggaaagctggattgtgcagggtgactttgaacctgaatcc | |
| | ggttatcgcgccatgcagcaaatcctgtcgcagccgcatcgccct | |
| | actgccgtcttctgtggtggcgatatcatggcaatgggcgcactt | |
| | tgtgctgctgatgaaatgggcctgcgcgtcccgcaggatgtttcg | |
| | ctgatcggttatgataacgtgcaacgcgcgctattttacgccggc | |
| | gctgaccacgatccatccatcagccaaaagattcgctgggtgaaa | |
| | cagcgttcaacatgctgttggatcgtatcgtcaacaaacgtgaag | |
| | aaccgcagtctattgaagtgcatccgcgcttgattgaacgccgct | |
| | ccgtggctgacggcccgttccgcgactatcgtcgttaa | |
| 34 | caatttctacaaaacacttgatactgtatgagcatacagtataa | RecA |
| | ttgcttcaacagaacatattgactatccggtattacccggcatg | locus |
| | acaggagtaaaa | in |
| | atggctatcgacgaaaacaaacagaaagcgttggcggcagcactgg | wildtype |
| | gccagattgagaaacaatttggtaaaggctccatcatgcgcctgg | E coli |
| | gtgaagaccgttccatggatgtggaaaccatctctaccggttcgc | MG1655 = |
| | tttcactggatatcgcgcttggggcaggtggtctgccgatgggcc | (sequence |
| | gtatcgtcgaaatctacggaccggaatcttccggtaaaaccacgc | upstream: |
| | tgacgctgcaggtgatcgccgcagcgcagcgtgaaggtaaaacct | Single |
| | gtgcgtttatcgatgctgaacacgcgctggacccaatctacgcac | underline; |
| | gtaaactgggcgtcgatatcgacaacctgctgtgctcccagccgg | downstream |
| | acaccggcgagcaggcactggaaatctgtgacgccctggcgcgtt | of |
| | ctggcgcagtagacgttatcgtcgttgactccgtggcggcactga | ORF: |
| | cgccgaaagcggaaatcgaaggcgaaatcggcgactctcacatgg | italics; |
| | gccttgcggcacgtatgatgagccaggcgatgcgtaagctggcgg | and |
| | gtaacctgaagcagtccaacacgctgctgatcttcatcaaccaga | ORF |
| | tccgtatgaaaattggtgtgatgttcggtaacccggaaaccacta | in |
| | ccggtggtaacgcgctgaaattctacgcctctgttcgtctcgaca | double |
| | tccgtcgtatcggcgcggtgaaagagggcgaaaacgtggtgggta | underline) |
| | gcgaaacccgcgtgaaagtggtgaagaacaaaatcgctgcgccgt | |
| | ttaaacaggctgaattccagatcctctacggcgaaggtatcaact | |
| | tctacggcgaactggttgacctgggcgtaaaagagaagctgatcg | |
| | agaaagcaggcgcgtggtacagctacaaaggtgagaagatcggtc | |
| | agggtaaagcgaatgcgactgcctggctgaaagataacccggaaa | |
| | ccgcgaaagagatcgagaagaaagtacgtgagttgctgctgagca | |
| | acccgaactcaacgccggatttctctgtagatgatagcgaaggcg | |
| | tagcagaaactaacgaagatttttaatcgtcttg | |
| | tttgatacacaagggtcgcatctgcggcccttttgcttttttaagtt | |
| | gtaaggatatgccatgacagaatcaacatcccgtcgcccggcata | |
| 35 | caatttctacaaaacacttgatactgtatgagcatacagta | RecA |
| | taattgcttcaacagaacatattgactatccggtattac | locus |
| | ccggcatgacaggagtaaaa | (entire |
| | tcgtcttgtttgatacacaagggtcgcatctgcggcccttt | ORF |
| | tgcttttttaagttgtaaggata | deleted) |
| | tgccatgacagaatcaacatcccgtcgcccggcata | in |
| | | strains |
| | | 1-4 |
| | | (sequence |
| | | upstream: |
| | | single |
| | | underline; |
| | | downstream |
| | | of |
| | | ORF: |
| | | italics) |
| 36 | aaataaccatctgaactatcaggaactttcctgatctggctg | EndA |
| | attgcataccaaaacagctttcgctacgttgctggctcgttt | locus |
| | taacacggagtaagtg | in |
| | atgtaccgttatttgtctattgctgcggtggtactgagcgcagcat | wildtype |
| | tttccggcccggcgttggccgaaggtatcaatagtttttctcagg | E coli |
| | cgaaagccgcggcggtaaaagtccacgctgacgcgcccggtacgt | MG1655 |
| | tttattgcggatgtaaaattaactggcagggcaaaaaaggcgttg | (sequence |
| | ttgatctgcaatcgtgcggctatcaggtgcgcaaaaatgaaaacc | upstream: |
| | gcgccagccgcgtagagtgggaacatgtcgttcccgcctggcagt | single |
| | tcggtcaccagcgccagtgctggcaggacggtggacgtaaaaact | underline; |
| | gcgctaaagatccggtctatcgcaagatggaaagcgatatgcata | and |
| | acctgcagccgtcagtcggtgaggtgaatggcgatcgcggcaact | ORF |
| | ttatgtacagccagtggaatggcggtgaaggccagtacggtcaat | in |
| | gcgccatgaaggtcgatttcaaagaaaaagctgccgaaccaccag | double |
| | cgcgtgcacgcggtgccattgcgcgcacctacttctatatgcgcg | underline) |
| | accaatacaacctgacactctctcgccagcaaacgcagctgttca | |
| | acgcatggaacaagatgtatccggttaccgactgggagtgcgagc | |
| | gcgatgaacgcatcgcgaaggtgcagggcaatcataacccgtatg | |
| | tgcaacgcgcttgccaggcgcgaaagagctaa | |
| 37 | aaataaccatctgaactatcaggaactttcctgatctggctg | endA |
| | attgcataccaaaacagctttcgctacgttgctggctc | locus |
| | gttttaacacggagtaagtgggttaccgactg | (Majority |
| | ggagtgcgagcgcgatgaacgcatcgcgaaggtgcagggcaatcataa | of |
| | cccgtatgtgcaacgcgcttgccaggcgcgaaagagctaa | ORF |
| | | deleted |
| | | to |
| | | implement |
| | | knockout) |
| | | in |
| | | strains |
| | | 1-4 |
| | | (sequence |
| | | upstream: |
| | | single |
| | | underline; |
| | | and |
| | | ORF |
| | | in |
| | | double |
| | | underline) |
| 38 | agagttgggcaacggatgtgctggtggaggtgatcgcctcctga | Mrr-hsdRMS- |
| | tgatgagccgctcccgatgtggtgtcgggagcgg | symRE-mcrBC |
| | tattttctataaaacttaccgc | locus |
| | tcactcaaaatagtccatatccagtttcggcaccttcaacaaacgt | in |
| | gaagaaacccctacttcgttttcgatcattaagtgcaccaggcgt | wildtype |
| | tccccatcaaccaacaccataccctcgacggattgggcaaagtca | E coli |
| | cgcgcctgagaagtaaatccagaagtggtaataaacaccccacgt | MG1655 |
| | ttcgctttttgcccagccagtgcgccgtaaaatgcctgtaattct | (sequence |
| | ggcctgcctacagtattctgccaacgttttgcctgaacataaact | upstream |
| | ttctccaggccaagtttatcaagcgatatcacaccatcgatgcca | (single |
| | ccatctccagtaccgccaacacgctgcaaatcatcacggtggccg | underline) |
| | ccataccccaggcgatgcaaaacatccagaacaatgacttcaaag | and |
| | cgcgaaggagaaacctgcaataagttttccagaacctcatcagcc | downstream |
| | accgcatcacgaagctcttttagcgcctgatctaaccgatcgtcc | (italics) |
| | gggctgctctttgcaagttcttcatgatcgggagagtcggctttc | of |
| | ggatctaaatcgacggcatccggccgtgacttaagtttgacattc | region |
| | acaaaagcgaaggccagatggttcgtctcctgctccgtcattggc | to |
| | tggggatgagacgcaacccagtcaaaacccgcaggagtcaggcac | be |
| | catttgccacgcgacaaactttgcgacaacccggcacgttttaaa | deleted) |
| | cggtcatgcgcccagcctgcacgatttttataaacaagttgtccg | |
| | ctggtaatgactttcgctcgctggctgtcatccagtcctaatgca | |
| | tccgcggcagcctcatgaacatcacgcgcggctgcaccttccggt | |
| | tttgttgccagataacgcagaacaggttcaataaatttgtcatag | |
| | gtaggaaccgtcatagtacatccttgcagaatcaggtagatgttt | |
| | ttcggctactatagcactacaaaaatagacgaacacgttagaaat | |
| | gagtcagttgctgtgaccgtggtcattgcccggaaaggtacagaa | |
| | agctaagatgagatgttatgggccttaaatatttggacaggcccg | |
| | cacagcaatggattaataacaatgatgaataaatccaattttgaa | |
| | ttcctgaagggcgtcaacgacttcacttatgccatcgcctgtgcg | |
| | gcggaaaataactacccggatgatcccaacacgacgctgattaaa | |
| | atgcgtatgtttggcgaagccacagcgaaacatcttggtctgtta | |
| | ctcaacatccccccttgtgagaatcaacacgatctcctgcgtgaa | |
| | ctcggcaaaatcgcctttgttgatgacaacatcctctctgtattt | |
| | cacaaattacgccgcattggtaaccaggcggtgcacgaatatcat | |
| | aacgatctcaacgatgcccagatgtgcctgcgactcgggttccgc | |
| | ctggctgtctggtactaccgtctggtcactaaagattatgacttc | |
| | ccggtgccggtgtttgtgttgccggaacgtggtgaaaacctctat | |
| | caccaggaagtgctgacgctaaaacaacagcttgaacagcaggtg | |
| | cgagaaaaagcgcagactcaggcagaagtcgaagcgcaacagcag | |
| | aagctggttgccctgaacggctatatcgccattctggaaggcaaa | |
| | cagcaggaaaccgaagcgcaaacccaggctcgccttgcggcactg | |
| | gaagcacagctcgccgagaagaacgcggaactggcaaaacagacc | |
| | gaacaggaacgtaaggcttaccacaaagaaattaccgatcaggcc | |
| | atcaagcgcacactcaaccttagcgaagaagagagtcgcttcctg | |
| | attgatgcgcaactgcgtaaagcaggctggcaggccgacagcaaa | |
| | accctgcgcttctccaaaggcgcacgtccggaacccggcgtcaat | |
| | aaagccattgccgaatggccgaccggaaaagatgaaacgggtaat | |
| | cagggctttgcggattatgtgctgtttgtcggcctcaaacccatc | |
| | gcggtggtagaggcgaaacgtaacaatatcgacgttcccgccagg | |
| | ctcaatgagtcgtatcgctacagtaaatgtttcgataatggcttc | |
| | ctgcgggaaaccttgcttgagcactactcaccggatgaagtgcat | |
| | gaagcagtgccagagtatgaaaccagctggcaggacaccagcggc | |
| | aaacaacggtttaaaatccccttctgctactcgaccaacgggcgc | |
| | gaataccgcgcaacaatgaagaccaaaagcggcatctggtatcgc | |
| | gacgtgcgtgatacccgcaatatgtcgaaagccttacccgagtgg | |
| | caccgcccggaagagctgctggaaatgctcggcagcgaaccgcaa | |
| | aaacagaatcagtggtttgccgataaccctggcatgagcgagctg | |
| | ggcctgcgttattatcaggaagatgccgtccgcgcggttgaaaag | |
| | gcaatcgtcaaggggcaacaagagatcctgctggcgatggcgacc | |
| | ggtaccggtaaaacccgtacggcaatcgccatgatgttccgcctg | |
| | atccagtcccagcgttttaaacgcattctcttccttgtcgaccgc | |
| | cgttctcttggcgaacaggcgctgggcgcgtttgaagatacgcgt | |
| | attaacggcgacaccttcaacagcattttcgacattaaagggctg | |
| | acggataaattcccggaagacagcaccaaaattcacgttgccacc | |
| | gtacagtcgctggtgaaacgcaccctgcaatcagatgaaccgatg | |
| | ccggtggcccgttacgactgtatcgtcgttgacgaagcgcatcgc | |
| | ggctatattctcgataaagagcagaccgaaggcgaactgcagttc | |
| | cgcagccagctggattacgtctctgcctaccgtcgcattctcgat | |
| | cacttcgatgcggtaaaaatcgctctcaccgccaccccggcgcta | |
| | catactgtgcagattttcggcgagccggtttaccgttatacctac | |
| | cgtaccgcggttatcgacggttttctgatcgaccaggatccgcct | |
| | attcagatcatcacccgcaacgcgcaggagggggtttatctctcc | |
| | aaaggcgagcaggtagagcgcatcagcccgcagggagaagtgatc | |
| | aatgacaccctggaagacgatcaggattttgaagtcgccgacttt | |
| | aaccgtggcctggtgatcccggcgtttaaccgcgccgtctgtaac | |
| | gaactcaccaattatcttgacccgaccggatcgcaaaaaacgctg | |
| | gtcttctgcgtcaccaatgcccatgccgatatggtggtggaagag | |
| | ctgcgtgccgcgttcaagaaaaagtatccgcaactggagcacgac | |
| | gcgatcatcaagatcaccggtgatgccgataaagacgcgcgcaaa | |
| | gtgcagaccatgatcacccgcttcaataaagagcggctgcccaat | |
| | atcgtggtaaccgtcgacctgctgacgaccggcgtcgatattccg | |
| | tcgatctgtaatatcgtgttcctgcgtaaagtacgcagccgcatt | |
| | ctgtacgaacagatgaaaggccgcgccacgcgcttatgcccggag | |
| | gtgaataaaaccagctttaagatttttgactgtgtcgatatctac | |
| | agcacgctggagagcgtcgacaccatgcgtccggtggtggtgcgc | |
| | ccgaaggtggaactgcaaacgctggtcaatgaaattaccgattca | |
| | gaaacctataaaatcaccgaagcggatggccgcagttttgccgag | |
| | cacagccatgaacaactggtggcgaagctccagcgtatcatcggt | |
| | ctggccacgtttaaccgtgaccgcagcgaaacgatagataaacag | |
| | gtgcgtcgtctggatgagctatgccaggacgcgggggcgtgaact | |
| | ttaacggcttcgcctcgcgcctgcgggaaaaagggccgcactgga | |
| | gcgccgaagtctttaacaaactgcctggctttatcgcccgtctgg | |
| | aaaagctgaaaacggacatcaacaacctgaatgatgcgccgatct | |
| | tcctcgatatcgacgatgaagtggtgagtgtaaaatcgctgtacg | |
| | gtgattacgacacgccgcaggatttcctcgaagcctttgactcgc | |
| | tggtgcaacgttccccgaacgcgcaaccggcattgcaggcagtta | |
| | ttaatcgcccgcgcgatctcacccgtaaagggctggtcgagctac | |
| | aggagtggtttgaccgccagcactttgaggaatcttccctgcgca | |
| | aagcatggaaagagacgcgcaatgaagatatcgccgcccggctga | |
| | ttggtcatattcgccgcgctgcggtgggcgatgcgctgaaaccgt | |
| | ttgaggaacgtgtcgatcacgcgctgacgcgcattaagggcgaaa | |
| | acgactggagcagcgagcaattaagctggctcgatcgtttagcgc | |
| | aggcgctgaaagagaaagtggtgctcgacgacgatgtcttcaaaa | |
| | ccggcaacttccaccgtcgcggcgggaaggcgatgctgcaaagaa | |
| | cctttgacgataatctcgataccctgctgggcaaattcagcgatt | |
| | atatctgggacgagctggcctgacacgtatacacttcatccttca | |
| | ggctgcctctgcgttggctgcgctcgttcaccccggtcacgtact | |
| | tctgtacgctcccggggattcactcacttgccgccttgatgcaac | |
| | ctgaatgattttgtgtatattaccctcggcaatttcttcttctgc | |
| | ggctcgatgaatttgggccgctgcttaatttacggaactcacaat | |
| | gaacaataacgatctggtcgcgaagctgtggaagctgtgcgacaa | |
| | cctgcgcgatggcggcgtttcctatcaaaactacgtcaatgaact | |
| | cgcctcgctgctgtttttgaaaatgtgtaaagagaccggtcagga | |
| | agcggaatacctgccggaaggttaccgctgggatgacctgaaatc | |
| | ccgcatcggccaggagcagttgcagttctaccgaaaaatgctcgt | |
| | gcatttaggcgaagatgacaaaaagctggtacaggcagtttttca | |
| | taatgttagtaccaccatcaccgagccgaaacaaataaccgcact | |
| | ggtcagcaatatggattcgctggactggtacaacggcgcgcacgg | |
| | taagtcgcgcgatgacttcggcgatatgtacgaagggctgttgca | |
| | gaagaacgcgaatgaaaccaagtctggtgcaggccagtacttcac | |
| | cccgcgtccgctgattaaaaccattattcatctgctgaaaccgca | |
| | gccgcgtgaagtggtgcaggacccggcggcaggtacggcgggctt | |
| | tttgattgaagccgaccgctatgttaagtcgcaaaccaatgatct | |
| | ggacgaccttgatggcgacacgcaggatttccagatccaccgcgc | |
| | gtttatcggcctcgaactggtgcccggcacccgtcgtctggcact | |
| | gatgaactgcctgctgcacgatattgaaggcaacctcgaccacgg | |
| | cggcgcaatccgtctgggcaacactctgggtagcgacggtgaaaa | |
| | cctgccgaaggcgcatattgtcgccactaacccgccgtttggcag | |
| | cgccgcaggcaccaacattacccgcacctttgttcacccgaccag | |
| | caacaaacagttgtgctttatgcagcatattatcgaaacgctgca | |
| | tcccggcggtcgtgcggcggtggtggtgccggataacgtgctgtt | |
| | tgaaggcggcaaaggcaccgacattcgtcgtgacctgatggataa | |
| | gtgtcatctgcacaccattctgcgtctgccgaccggtatttttta | |
| | cgctcagggcgtgaagaccaacgtgctgttctttaccaaagggac | |
| | ggtggcgaacccgaatcaggataagaactgtaccgatgatgtgtg | |
| | ggtgtatgacctgcgtaccaatatgccgagtttcggcaagcgcac | |
| | accgtttaccgacgagcatttgcagccgtttgagcgcgtgtatgg | |
| | cgaagacccgcacggtttaagcccgcgcactgaaggtgaatggag | |
| | ttttaacgccgaagagacggaagttgccgacagcgaagagaacaa | |
| | aaacaccgaccagcatcttgctaccagccgctggcgcaagttcag | |
| | ccgtgagtggatccgcaccgcaaaatccgattcgctggatatctc | |
| | ctggctgaaagataaagacagtattgatgccgacagcctgccgga | |
| | gccggatgtattagcggcagaagcgatgggcgaactggtacaggc | |
| | gctgtctgaactggatgcgctgatgcgtgaactgggggcgagcga | |
| | tgaggccgatttgcagcgtcagttgctggaagaagcgtttggtgg | |
| | ggtgaaggaatgagtgcggggaaattgccggaggggtgggttatc | |
| | gccccagtatctacggtcacaactctaatccgaggagtaacgtat | |
| | aaaaaagagcaggcaataaattatctaaaagatgattatttgcct | |
| | cttatccgtgcgaacaatattcagaatggcaagtttgatactacg | |
| | gacttggtttttgttcctaaaaatcttgttaaagaaagtcaaaaa | |
| | atatctcctgaagatattgttattgcaatgtcatcagggagcaaa | |
| | tccgtagttggtaaatccgcacatcagcatctaccatttgaatgt | |
| | agtttcggcgcattttgcggtgtattacgtcctgaaaaacttata | |
| | ttttctggttttattgctcatttcacaaaatcttctctttatcga | |
| | aacaaaatttcatcactttctgctggtgcaaatattaataatatt | |
| | aagccggcaagctttgatttgataaatataccaatcccaccactt | |
| | gccgaacaaaaaatcatcgctgaaaaactcgatacgctgctggcg | |
| | caggtagacagcaccaaagcacgttttgagcaaatcccacaaatc | |
| | ctgaaacgttttcgtcaagcggtattggggggcgcagttaatgga | |
| | aaattgacagaaaaatggcgtaattttgagccgcaacattctgta | |
| | tttaagaagttaaattttgaatctatcttaactgaattacgtaat | |
| | ggtctttcatcaaagccaaatgaaagtggtgttggtcatccaata | |
| | ctacgcattagttctgtacgtgctggccatgtagatcaaaacgat | |
| | attcggtttctagaatgttcagaaagtgaactaaaccgccacaaa | |
| | ttacaagatggagatcttttatttactcgctataacggaagttta | |
| | gaatttgttggtgtttgtgggttattgaaaaaattacaacatcaa | |
| | aatttgctatatcctgataaacttattcgagctcgattaaccaaa | |
| | gatgctttaccagaatatatcgaaatatttttttcatccccctca | |
| | gcacgaaatgcaatgatgaactgcgtgaaaacaacttctggtcaa | |
| | aaaggtatttcaggaaaagatatcaaatcccaagttgttttatta | |
| | cctccagtaaaagaacaagccgaaatcgttcgccgcgtcgagcaa | |
| | ctcttcgcctacgccgacaccatagaaaaacaggtcaacaacgcc | |
| | ttagcccgcgtcaacaacctgacgcaatccatcctggcaaaagcg | |
| | ttccgtggtgaacttaccgcccagtggcgggccgaaaacccggat | |
| | ttgatcagcggagaaaacagcgccgccgcgttgctggaaaaaatc | |
| | aaagctgaacgcgcagctagcgggggtaaaaaagcctcacgtaaa | |
| | aaatcctgaacattattttctggcgcacctttccggtgcgctttt | |
| | tattatttcacgccaatcataacccacataaatatatttaaatca | |
| | ttccagaaattgcccattttattctatttttagctggactttccc | |
| | catatttactgatgatatatacaggtatttagcgcggtgcggatg | |
| | tgcgccaacacacccgcatcgctaatcacaatcactattcctgga | |
| | gaatagcagttatgactgacacgcattctattgcacaaccgttcg | |
| | aagcagaagtctccccggcaaataaccgtcatgtcaccgtcggtt | |
| | atgcgagtcgctacccggattacagccgtattcccgccatcaccc | |
| | tgaaaggtcagtggctggaagccgccggttttgccactggcacgg | |
| | cggtagatgtcaaagtgatggaaggctgtattgtcctcaccgccc | |
| | aaccacccgccgccgaggagagcgaactgatgcagtcgctacgcc | |
| | aggtgtgcaaactgtcggcgcgtaaacaaaagcaggtgcaggcgt | |
| | ttattggagtgattgccggtaaacagaaagtcgcgtaacagcgtt | |
| | tactcccggttaaccaccgggagccttccactgactcaatagaaa | |
| | ctttccccctcagtaaatatttaccagtctgattttgcagtaaaa | |
| | atctattgtttcagtacgttgcgaaagcgataatagaggcttagc | |
| | aatgaggaaggcatatcttatggaatctattcaaccctggattga | |
| | aaaatttattaagcaagcacagcaacaacgttcgcaatccactaa | |
| | agattatccaacgtcttaccgtaacctgcgagtaaaattgagttt | |
| | cggttatggtaattttacgtctattccctggtttgcatttcttgg | |
| | agaaggtcaggaagcttctaacggtatatatcccgttattctcta | |
| | ttataaagattttgatgagttggttttggcttatggtataagcga | |
| | cacgaatgaaccacatgcccaatggcagttctcttcagacatacc | |
| | taaaacaatcgcagagtattttcaggcaacttcgggtgtatatcc | |
| | taaaaaatacggacagtcctattacgcctgttcccaaaaagtctc | |
| | acagggtattgattacacccgatttgcctctatgctggacaacat | |
| | aatcaacgactataaattaatatttaattctggcaagagtgttat | |
| | tccacctatgtcaaaaactgaatcatactgtctggaagatgcgtt | |
| | aaatgatttgtttatccctgaaaccacaatagagacgatactcaa | |
| | acgattaaccatcaaaaaaaatattatcctccaggggccgcccgg | |
| | cgttggaaaaacctttgttgcacgccgtctggcttacttgctgac | |
| | aggagaaaaggctccgcaacgcgtcaatatggttcagttccatca | |
| | atcttatagctatgaggattttatacagggctatcgtccgaatgg | |
| | cgtcggcttccgacgtaaagacggcatattttacaatttttgtca | |
| | gcaagctaaagagcagccagagaaaaagtatatttttattataga | |
| | tgaaatcaatcgtgccaatctcagtaaagtatttggcgaagtgat | |
| | gatgttaatggaacatgataaacgaggtgaaaactggtctgttcc | |
| | cctaacctactccgaaaacgatgaagaacgattctatgtcccgga | |
| | gaatgtttatatcatcggtttaatgaatactgccgatcgctctct | |
| | ggccgttgttgactatgccctacgcagacgattttctttcataga | |
| | tattgagccaggttttgatacaccacagttccggaattttttact | |
| | gaataaaaaagcagaaccttcatttgttgagtctttatgccaaaa | |
| | aatgaacgagttgaaccaggaaatcagcaaagaggccactatcct | |
| | tgggaaaggattccgcattgggcatagttacttctgctgtgggtt | |
| | ggaagatggcacctctccggatacgcaatggcttaatgaaattgt | |
| | gatgacggatatcgcccctttactcgaagaatatttctttgatga | |
| | cccctataaacaacagaaatggaccaacaaattattaggggactc | |
| | atagtggaacagcccgtgatacctgtccgtaatatctattacatg | |
| | cttacctatgcatggggttatttacaggaaattaagcaggcaaac | |
| | cttgaagccatacccggtaacaatcttcttgatatcctggggtat | |
| | gtattaaataaaggggttttacagctttcacgccgagggcttgag | |
| | cttgattacaatcctaacaccgagatcattcctggcatcaaaggg | |
| | cgaatagagtttgctaaaacaatacgcggcttccatcttaatcat | |
| | gggaaaaccgtcagtacttttgatatgcttaatgaagacacgctg | |
| | gctaaccgaattataaaaagcacattagccatattaattaagcat | |
| | gaaaagttaaattcaactatcagagatgaagctcgttcactttat | |
| | agaaaattaccgggcattagcactcttcatttaactccgcagcat | |
| | ttcagctatctgaatggcggaaaaaatacgcgttattataaattc | |
| | gttatcagtgtctgcaaattcatcgtcaataattctattccaggt | |
| | caaaacaaaggacactaccgtttctatgattttgaaagaaacgaa | |
| | aaagagatgtcattactttatcaaaagtttctttatgaattttgc | |
| | cgtcgtgaattaacgtctgcaaacacaacccgctcttatttaaaa | |
| | tgggatgcatcgagtatatcggatcagtcacttaatttgttacct | |
| | cgaatggaaactgacatcaccattcgctcatcagaaaaaatactt | |
| | atcgttgacgccaaatactataagagcattttttcacgacgaatg | |
| | ggaacagaaaaatttcattcgcaaaatctttatcaactgatgaat | |
| | tacttatggtcgttaaagcctgaaaatggcgaaaacatagggggg | |
| | ttattaatatatccccacgtagataccgcagtgaaacatcgttat | |
| | aaaattaatggcttcgatattggcttgtgtaccgtcaatttaggt | |
| | caggaatggccgtgtatacatcaagaattactcgacattttcgat | |
| | gaatatctcaaataa | |
| | aatatcaggccggatgcggctgcgccttatccggcc | |
| | cataaccccttacttcctcaaccccgcaaacgcagcccga | |
| | atctcttcctccggcagctggatc | |
| 39 | caatttctacaaaacacttgatactgtatgagcataca | |
| | gtataattgcttcaacagaacatattgactatccggta | |
| | ttacccggcatgacaggagtaaaa | |
| | atggctatcgacgaaaacaaacagaaagcgttggcggcagcactgg | |
| | gccagattgagaaacaatttggtaaaggctccatcatgcgcctgg | |
| | gtgaagaccgttccatggatgtggaaaccatctctaccggttcgc | |
| | tttcactggatatcgcgcttggggcaggtggtctgccgatgggcc | |
| | gtatcgtcgaaatctacggaccggaatcttccggtaaaaccacgc | |
| | tgacgctgcaggtgatcgccgcagcgcagcgtgaaggtaaaacct | |
| | gtgcgtttatcgatgctgaacacgcgctggacccaatctacgcac | |
| | gtaaactgggcgtcgatatcgacaacctgctgtgctcccagccgg | |
| | acaccggcgagcaggcactggaaatctgtgacgccctggcgcgtt | Mrr-hsdRMS- |
| | ctggcgcagtagacgttatcgtcgttgactccgtggcggcactga | symRE-mcrBC |
| | cgccgaaagcggaaatcgaaggcgaaatcggcgactctcacatgg | locus |
| | gccttgcggcacgtatgatgagccaggcgatgcgtaagctggcgg | (deletion) |
| | gtaacctgaagcagtccaacacgctgctgatcttcatcaaccaga | (sequence |
| | tccgtatgaaaattggtgtgatgttcggtaacccggaaaccacta | upstream: |
| | ccggtggtaacgcgctgaaattctacgcctctgttcgtctcgaca | single |
| | tccgtcgtatcggcgcggtgaaagagggcgaaaacgtggtgggta | underline; |
| | gcgaaacccgcgtgaaagtggtgaagaacaaaatcgctgcgccgt | downstream |
| | ttaaacaggctgaattccagatcctctacggcgaaggtatcaact | of |
| | tctacggcgaactggttgacctgggcgtaaaagagaagctgatcg | ORF: |
| | agaaagcaggcgcgtggtacagctacaaaggtgagaagatcggtc | italics; |
| | agggtaaagcgaatgcgactgcctggctgaaagataacccggaaa | and |
| | ccgcgaaagagatcgagaagaaagtacgtgagttgctgctgagca | ORF |
| | acccgaactcaacgccggatttctctgtagatgatagcgaaggcg | in |
| | tagcagaaactaacgaagatttttaatcgtctt | double |
| | gtttgatacacaagggtcgcatctgcggcccttttgcttttttaag | underline) |
| | ttgtaaggatatgccatgacagaatcaacatcccgtcgcccggcata | |
| 40 | agagttgggcaacggatgtgctggtggaggtgatcgcctcctg | Mrr- |
| | atgatgagccgctcccgatgtggtgtcgggagcggtattttct | hsdRMS- |
| | ataaaacttaccgc [symbol] | symRE- |
| | TACTAGAGAAAGAGGAGAAG | mcrBC |
| | [symbol] | locus |
| | [symbol] | (replaced |
| | [symbol] | with |
| | [symbol] | prsA* |
| | [symbol] | expression |
| | [symbol] | cassette) |
| | [symbol] | in |
| | [symbol] | strain |
| | [symbol] | 3 |
| | [symbol] | and |
| | [symbol] | 4 |
| | [symbol] | Upstream, |
| | [symbol] | unaltered |
| | [symbol] | genomic |
| | [symbol] [symbol] [symbol] | region = |
| | [symbol] [symbol] | single |
| | [symbol] [symbol] | underline; |
| | [symbol] [symbol] | J23119 |
| | [symbol] [symbol] | promoter = |
| | [symbol] [symbol] | double |
| | [symbol] [symbol] | underline; |
| | [symbol] [symbol] | Upstream |
| | [symbol] [symbol] | untranslated |
| | [symbol] [symbol] | region |
| | [symbol] [symbol] | containing |
| | [symbol] | RBS = |
| | cgcaaaaaaccccgcttcggggggttttttcgc | single |
| | aatatcaggccggatgcggctgcgccttatcc | underline |
| | ggcccataaccccttacttcctcaaccccgca | and |
| | aacgcagcccgaatctcttcctccggcagctg | italics; |
| | gatc | prsA* |
| | | open |
| | | reading |
| | | frame = |
| | | double |
| | | underline |
| | | and |
| | | italics; |
| | | transcriptional |
| | | terminator |
| | | Bba_b1002 |
| | | terminator; |
| | | and |
| | | Downstream, |
| | | unaltered |
| | | genomic |
| | | region = |
| | | italics |

*Unless otherwise specified, sequences are depicted and listed, and are to be read :- 5′-to-3′ for nucleotide sequences; and- N-terminus to C-terminus for amino acid sequences.
**Unless otherwise specified, NT denotes nucleotide sequences and AA denotes amino acid sequences

All references cited herein are fully incorporated by reference. Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### OTHER EMBODIMENTS

Embodiment 1. An engineered nucleic acid vector comprising a stationary-phase-induced promoter and a primosome assembly site (PAS).

Embodiment 2. The engineered nucleic acid vector of embodiment 1, further comprising point-mutations causing the formation of a critical stem-loop on RNAII, SL4.

Embodiment 3. The engineered nucleic acid vector of embodiment 1 or 2, wherein a native promoter for RNAII has been disrupted.

Embodiment 4. The engineered nucleic acid vector of embodiment 1 or 2, wherein a native promoter for RNAII has been deleted.

Embodiment 5. The engineered nucleic acid vector of embodiment 1 or any one of embodiments 2-4, wherein the stationary-phase-induced promoter is P(osmY).

Embodiment 6. The engineered nucleic acid vector of embodiment 5, wherein the P(osmY) has a sequence of SEQ ID NO: 27.

Embodiment 7. The engineered nucleic acid vector of any one of embodiments 1-6, wherein the PAS has a sequence of SEQ ID NO: 28.

Embodiment 8. The engineered nucleic acid vector of embodiment 2 or any one of embodiments 3-7, wherein the SL4 has a sequence of SEQ ID NO: 29.

Embodiment 9. The engineered nucleic acid vector of embodiment 8, wherein the vector is Plasmid 1 (+PAS+P(osmY)).

Embodiment 10. The engineered nucleic acid vector of embodiment 8 or embodiment 9, wherein the vector is Plasmid 2 (+PAS+P(osmY)+SL4).

Embodiment 11. The engineered nucleic acid vector of embodiment 1, wherein the vector has a sequence of at least 70% sequence identity to SEQ ID NO: 19.

Embodiment 12. The engineered nucleic acid vector of embodiment 1, wherein the vector has a sequence of at least 70% sequence identity to SEQ ID NO: 20.

Embodiment 13. The engineered nucleic acid vector of any one of embodiments 1-12, comprising in the following 5′ to 3′ configuration: (a) an origin of replication; (b) the promoter; and (c) an antibiotic resistance gene.

Embodiment 14. The engineered nucleic acid vector of any one of embodiments 1-13, further comprising an open reading frame (ORF) encoding an mRNA of interest.

Embodiment 15. A recombinant plasmid comprising the geneotype:|<repA|ori_ts|recA|<bla|<tetR|<P(tetR)|P(tet)>|gamma>|beta>|exo>|a>|.

Embodiment 16. A recombinant plasmid comprising a nucleic acid sequence with at least 70% identity to SEQ ID NO: 19.

Embodiment 17. A recombinant plasmid comprising a nucleic acid sequence with at least 70% identity to SEQ ID NO: 20.

Embodiment 18. A method of performing an in vitro transcription reaction using the engineered nucleic acid vector of any one of embodiments 1-17.

Embodiment 19. A nucleic acid comprising a prsA variant.

Embodiment 20. The nucleic acid of embodiment 19, wherein the nucleic acid has 70%-99% sequence identity to prsA* (SEQ ID NO: 23).

Embodiment 21. The nucleic acid of embodiment 19, wherein the nucleic acid has at least 70% sequence identity to prsA* (SEQ ID NO: 23)

Embodiment 22. The nucleic acid of embodiment 19, wherein the nucleic acid has at least 80%, 90%, or 95% sequence identity to prsA* (SEQ ID NO: 23).

Embodiment 23. The nucleic acid of embodiment 19, wherein the nucleic acid encodes a protein having at least 95% sequence identity to prsA* (SEQ ID NO: 24).

Embodiment 24. The nucleic acid of embodiment 19, wherein the nucleic acid has 100% sequence identity to SEQ ID NO: 23 or encodes a protein having 100% sequence identity to SEQ ID NO: 24.

Embodiment 25. A genetically modified microorganism comprising a prsA variant, wherein the microorganism has a genome in which a repressor gene purR has been disrupted.

Embodiment 26. The genetically modified microorganism of embodiment 25, wherein the prsA variant has 70%-99% sequence identity to prsA.

Embodiment 27. The genetically modified microorganism of embodiment 25, wherein the prsA variant has least 90% sequence identity to prsA* (SEQ ID NO: 23).

Embodiment 28. The genetically modified microorganism of embodiment 25, wherein the prsA variant comprises a sequence of SEQ ID NO: 23.

Embodiment 29. The genetically modified microorganism of any one of embodiments 25-28, wherein the purR has been deleted.

Embodiment 30. The genetically modified microorganism of embodiment 29, wherein the purR comprises a sequence of SEQ ID NO: 25.

Embodiment 31. The genetically modified microorganism of any one of embodiments 25-30, wherein an EcoKI restriction system has been deleted from the genome.

Embodiment 32. The genetically modified microorganism of any one of embodiments 25-31, wherein endA has been deleted from the genome.

Embodiment 33. The genetically modified microorganism of any one of embodiments 25-32, wherein recA has been deleted from the genome.

Embodiment 34. The genetically modified microorganism of any one of embodiments 25-33, wherein the genetically modified microorganism is a recombinant strain of Escherichia coli (E. coli).

Embodiment 35. A recombinant strain of Escherichia coli (E. coli), comprising: an E. coli genome with at least the following gene deletions: endA (ΔendA) and recA (ΔrecA).

Embodiment 36. The recombinant strain of embodiment 35, wherein the E. coli is derived from MG1655.

Embodiment 37. The recombinant strain of embodiment 35 or embodiment 36, wherein the E. coli genome comprises a nucleic acid sequence of MG1655 genome including at least the following gene deletions: endA (ΔendA) and recA (ΔrecA) with respect to the MG1655 genome.

Embodiment 38. The recombinant strain of embodiment 35 or any one of embodiments 36-37, wherein the E. coli genome comprises a nucleic acid sequence of at least 95% sequence identity with MG1655 genome.

Embodiment 39. The recombinant strain of any one of embodiment 35-38, wherein an EcoKI restriction system has been deleted from the genome of the E. coli.

Embodiment 40. The recombinant strain of embodiment 39, wherein the E. coli genome comprises a nucleic acid sequence with at least 80% identity to MG1655 genome.

Embodiment 41. The recombinant strain of embodiment 39 or embodiment 40, wherein the E. coli genome comprises a nucleic acid sequence of wherein the E. coli genome comprises a nucleic acid sequence of MG1655 genome including the EcoKI restriction system deletion with respect to the MG1655 genome.

Embodiment 42. The recombinant strain of any one of embodiment 35-41, wherein the E. coli comprises a prsA variant.

Embodiment 43. The recombinant strain of embodiment 42, wherein the E. coli genome comprises a nucleic acid sequence with at least 80% identity to MG1655 genome.

Embodiment 44. The recombinant strain of embodiment 43, wherein the E. coli genome comprises a nucleic acid sequence of SEQ ID NO: 23.

Embodiment 45. The recombinant strain of any one of embodiment 35-44, wherein a purR sequence has been deleted from the genome of the E. coli.

Embodiment 46. The recombinant strain of embodiment 45, wherein the E. coli genome comprises a nucleic acid sequence with at least 80% identity to MG1655 genome.

Embodiment 47. The recombinant strain of embodiment 46, wherein the E. coli genome has a nucleic acid sequence of SEQ ID NO: 25 deleted with respect to the MG1655 genome.

Embodiment 48. The recombinant strain of any one of embodiment 35-47, wherein the E. coli genome further comprises: at least one of gene deletions selected from the group comprising: mrr; hsdR; hsdM; hsdS; symE; and mcrBC.

Embodiment 49. The recombinant strain of any one of embodiment 35-48, the E. coli genome is derived from the strain MG or KS.

Embodiment 50. A genetically modified microorganism comprising Strain 3.

Embodiment 51. A genetically modified microorganism comprising Strain 4.

Embodiment 52. An engineered nucleic acid vector comprising a nucleic acid having at least 70% sequence identity to SEQ ID NO: 21.

Embodiment 53. An engineered nucleic acid vector comprising a nucleic acid having at least 80% sequence identity to SEQ ID NO: 21.

Embodiment 54. An engineered nucleic acid vector comprising a nucleic acid having at least 90% sequence identity to SEQ ID NO: 21.

Embodiment 55. An engineered nucleic acid vector comprising a nucleic acid having at least 95% sequence identity to SEQ ID NO: 21.

Embodiment 56. An engineered nucleic acid vector comprising a nucleic acid having SEQ ID NO: 21.

Embodiment 57. An engineered nucleic acid vector comprising a nucleic acid having at least 70% sequence identity to SEQ ID NO: 22.

Embodiment 58. An engineered nucleic acid vector comprising a nucleic acid having at least 80% sequence identity to SEQ ID NO: 22.

Embodiment 59. An engineered nucleic acid vector comprising a nucleic acid having at least 90% sequence identity to SEQ ID NO: 22.

Embodiment 60. An engineered nucleic acid vector comprising a nucleic acid having at least 95% sequence identity to SEQ ID NO: 22.

Embodiment 61. An engineered nucleic acid vector comprising a nucleic acid having SEQ ID NO: 22.

Embodiment 62. An engineered nucleic acid vector comprising a nucleic acid sequence having at least 70% sequence identity to any one of SEQ ID NO: 1-15.

Embodiment 63. An engineered nucleic acid vector comprising a nucleic acid sequence having at least 80% sequence identity to any one of SEQ ID NO: 1-15.

Embodiment 64. An engineered nucleic acid vector comprising a nucleic acid sequence having at least 90% sequence identity to any one of SEQ ID NO: 1-15.

Embodiment 65. An engineered nucleic acid vector comprising a nucleic acid sequence having at least 95% sequence identity to any one of SEQ ID NO: 1-15.

Embodiment 66. An engineered nucleic acid vector comprising a nucleic acid sequence having at least 99% sequence identity to any one of SEQ ID NO: 1-15.

Embodiment 67. An engineered nucleic acid vector comprising a nucleic acid sequence of any one of SEQ ID NO: 1-15.

Embodiment 68. An engineered nucleic acid vector comprising a nucleic acid sequence having at least 70% sequence identity to SEQ ID NO: 10.

Embodiment 69. An engineered nucleic acid vector comprising a nucleic acid sequence having at least 70% sequence identity to SEQ ID NO: 11.

Embodiment 70. An engineered nucleic acid vector comprising a nucleic acid sequence having at least 95% sequence identity to SEQ ID NO: 10.

Embodiment 71. An engineered nucleic acid vector comprising a nucleic acid sequence having at least 95% sequence identity to SEQ ID NO: 11.

Embodiment 72. An engineered nucleic acid vector comprising a nucleic acid sequence having at least 99% sequence identity to SEQ ID NO: 10.

Embodiment 73. An engineered nucleic acid vector comprising a nucleic acid sequence having at least 99% sequence identity to SEQ ID NO: 11.

Embodiment 74. An engineered nucleic acid vector comprising a nucleic acid sequence of SEQ ID NO: 10.

Embodiment 75. An engineered nucleic acid vector comprising a nucleic acid sequence of SEQ ID NO: 11.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

What is claimed is:

1. An engineered nucleic acid vector comprising a stationary-phase-induced promoter and a primosome assembly site (PAS).

2. The engineered nucleic acid vector of claim 1, further comprising point-mutations causing the formation of a critical stem-loop on RNAII, SL4.

3. The engineered nucleic acid vector of claim 1 or 2, wherein a native promoter for RNAII has been disrupted.

4. The engineered nucleic acid vector of claim 1 or 2, wherein a native promoter for RNAII has been deleted.

5. The engineered nucleic acid vector of claim 1 or any one of claims 2-4, wherein the stationary-phase-induced promoter is P(osmY).

6. The engineered nucleic acid vector of claim 5, wherein the P(osmY) has a sequence of SEQ ID NO: 27.

7. The engineered nucleic acid vector of any one of claims 1-6, wherein the PAS has a sequence of SEQ ID NO: 28.

8. The engineered nucleic acid vector of claim 2 or any one of claims 3-7, wherein the SL4 has a sequence of SEQ ID NO: 29.

9. The engineered nucleic acid vector of claim 8, wherein the vector is Plasmid 1 (+PAS+P(osmY)).

10. The engineered nucleic acid vector of claim 8 or claim 9, wherein the vector is Plasmid 2 (+PAS+P(osmY)+SL4).

11. The engineered nucleic acid vector of claim 1, wherein the vector has a sequence of at least 70% sequence identity to SEQ ID NO: 19.

12. The engineered nucleic acid vector of claim 1, wherein the vector has a sequence of at least 70% sequence identity to SEQ ID NO: 20.

13. The engineered nucleic acid vector of any one of claims 1-12, comprising in the following 5′ to 3′ configuration:
(a) an origin of replication;
(b) the promoter; and
(c) an antibiotic resistance gene.

14. The engineered nucleic acid vector of any one of claims 1-13, further comprising an open reading frame (ORF) encoding an mRNA of interest.

15. A recombinant plasmid comprising the geneotype:|<repA|ori_ts|<recA|<bla|<tetR|<P(tetR)|P(tet)>|gamma>|beta>|exo>|a>|.

16. A recombinant plasmid comprising a nucleic acid sequence with at least 70% identity to SEQ ID NO: 19.

17. A recombinant plasmid comprising a nucleic acid sequence with at least 70% identity to SEQ ID NO: 20.

18. A method of performing an in vitro transcription reaction using the engineered nucleic acid vector of any one of claims 1-17.

19. A nucleic acid comprising a prsA variant.

20. The nucleic acid of claim 19, wherein the nucleic acid has 70%-99% sequence identity to prsA* (SEQ ID NO: 23).

21. The nucleic acid of claim 19, wherein the nucleic acid has at least 70% sequence identity to prsA* (SEQ ID NO: 23)

22. The nucleic acid of claim 19, wherein the nucleic acid has at least 80%, 90%, or 95% sequence identity to prsA* (SEQ ID NO: 23).

23. The nucleic acid of claim 19, wherein the nucleic acid encodes a protein having at least 95% sequence identity to prsA* (SEQ ID NO: 24).

24. The nucleic acid of claim 19, wherein the nucleic acid has 100% sequence identity to SEQ ID NO: 23 or encodes a protein having 100% sequence identity to SEQ ID NO: 24.

25. A genetically modified microorganism comprising a prsA variant, wherein the microorganism has a genome in which a repressor gene purR has been disrupted.

26. The genetically modified microorganism of claim 25, wherein the prsA variant has 70%-99% sequence identity to prsA.

27. The genetically modified microorganism of claim 25, wherein the prsA variant has least 90% sequence identity to prsA* (SEQ ID NO: 23).

28. The genetically modified microorganism of claim 25, wherein the prsA variant comprises a sequence of SEQ ID NO: 23.

29. The genetically modified microorganism of any one of claims 25-28, wherein the purR has been deleted.

30. The genetically modified microorganism of claim 29, wherein the purR comprises a sequence of SEQ ID NO: 25.

31. The genetically modified microorganism of any one of claims 25-30, wherein an EcoKI restriction system has been deleted from the genome.

32. The genetically modified microorganism of any one of claims 25-31, wherein endA has been deleted from the genome.

33. The genetically modified microorganism of any one of claims 25-32, wherein recA has been deleted from the genome.

34. The genetically modified microorganism of any one of claims 25-33, wherein the genetically modified microorganism is a recombinant strain of Escherichia coli (E. coli).

35. A recombinant strain of Escherichia coli (E. coli), comprising: an E. coli genome with at least the following gene deletions: endA (ΔendA) and recA (ΔrecA).

36. The recombinant strain of claim 35, wherein the E. coli is derived from MG1655.

37. The recombinant strain of claim 35 or claim 36, wherein the E. coli genome comprises a nucleic acid sequence of MG1655 genome including at least the following gene deletions: endA (ΔendA) and recA (ΔrecA) with respect to the MG1655 genome.

38. The recombinant strain of claim 35 or any one of claims 36-37, wherein the E. coli genome comprises a nucleic acid sequence of at least 95% sequence identity with MG1655 genome.

39. The recombinant strain of any one of claim 35-38, wherein an EcoKI restriction system has been deleted from the genome of the E. coli.

40. The recombinant strain of claim 39, wherein the E. coli genome comprises a nucleic acid sequence with at least 80% identity to MG1655 genome.

41. The recombinant strain of claim 39 or claim 40, wherein the E. coli genome comprises a nucleic acid sequence of wherein the E. coli genome comprises a nucleic acid sequence of MG1655 genome including the EcoKI restriction system deletion with respect to the MG1655 genome.

42. The recombinant strain of any one of claim 35-41, wherein the E. coli comprises a prsA variant.

43. The recombinant strain of claim 42, wherein the E. coli genome comprises a nucleic acid sequence with at least 80% identity to MG1655 genome.

44. The recombinant strain of claim 43, wherein the E. coli genome comprises a nucleic acid sequence of SEQ ID NO: 23.

45. The recombinant strain of any one of claim 35-44, wherein a purR sequence has been deleted from the genome of the E. coli.

46. The recombinant strain of claim 45, wherein the E. coli genome comprises a nucleic acid sequence with at least 80% identity to MG1655 genome.

47. The recombinant strain of claim 46, wherein the E. coli genome has a nucleic acid sequence of SEQ ID NO: 25 deleted with respect to the MG1655 genome.

48. The recombinant strain of any one of claim 35-47, wherein the E. coli genome further comprises: at least one of gene deletions selected from the group comprising: mrr; hsdR; hsdM; hsdS; symE; and mcrBC.

49. The recombinant strain of any one of claim 35-48, the E. coli genome is derived from the strain MG or KS.

50. A genetically modified microorganism comprising Strain 3.

51. A genetically modified microorganism comprising Strain 4.

52. An engineered nucleic acid vector comprising a nucleic acid having at least 70% sequence identity to SEQ ID NO: 21.

53. An engineered nucleic acid vector comprising a nucleic acid having at least 80% sequence identity to SEQ ID NO: 21.

54. An engineered nucleic acid vector comprising a nucleic acid having at least 90% sequence identity to SEQ ID NO: 21.

55. An engineered nucleic acid vector comprising a nucleic acid having at least 95% sequence identity to SEQ ID NO: 21.

56. An engineered nucleic acid vector comprising a nucleic acid having SEQ ID NO: 21.

57. An engineered nucleic acid vector comprising a nucleic acid having at least 70% sequence identity to SEQ ID NO: 22.

58. An engineered nucleic acid vector comprising a nucleic acid having at least 80% sequence identity to SEQ ID NO: 22.

59. An engineered nucleic acid vector comprising a nucleic acid having at least 90% sequence identity to SEQ ID NO: 22.

60. An engineered nucleic acid vector comprising a nucleic acid having at least 95% sequence identity to SEQ ID NO: 22.

61. An engineered nucleic acid vector comprising a nucleic acid having SEQ ID NO: 22.

62. An engineered nucleic acid vector comprising a nucleic acid sequence having at least 70% sequence identity to any one of SEQ ID NO: 1-15.

63. An engineered nucleic acid vector comprising a nucleic acid sequence having at least 80% sequence identity to any one of SEQ ID NO: 1-15.

64. An engineered nucleic acid vector comprising a nucleic acid sequence having at least 90% sequence identity to any one of SEQ ID NO: 1-15.

65. An engineered nucleic acid vector comprising a nucleic acid sequence having at least 95% sequence identity to any one of SEQ ID NO: 1-15.

66. An engineered nucleic acid vector comprising a nucleic acid sequence having at least 99% sequence identity to any one of SEQ ID NO: 1-15.

67. An engineered nucleic acid vector comprising a nucleic acid sequence of any one of SEQ ID NO: 1-15.

68. An engineered nucleic acid vector comprising a nucleic acid sequence having at least 70% sequence identity to SEQ ID NO: 10.

69. An engineered nucleic acid vector comprising a nucleic acid sequence having at least 70% sequence identity to SEQ ID NO: 11.

70. An engineered nucleic acid vector comprising a nucleic acid sequence having at least 95% sequence identity to SEQ ID NO: 10.

71. An engineered nucleic acid vector comprising a nucleic acid sequence having at least 95% sequence identity to SEQ ID NO: 11.

72. An engineered nucleic acid vector comprising a nucleic acid sequence having at least 99% sequence identity to SEQ ID NO: 10.

73. An engineered nucleic acid vector comprising a nucleic acid sequence having at least 99% sequence identity to SEQ ID NO: 11.

74. An engineered nucleic acid vector comprising a nucleic acid sequence of SEQ ID NO: 10.

75. An engineered nucleic acid vector comprising a nucleic acid sequence of SEQ ID NO: 11.
